SEQUENCE LISTING (8 sequences)

SEQ ID NO: 1 - DNA, 20 bp, Artificial Sequence
  A [gamma-32P]-ATP 5′-end labeled 20 nucleotide DNA primer
tcgggcgccactgctagaga

SEQ ID NO: 2 - DNA, 57 bp, Artificial Sequence
  DNA template for HIV1-RT
ctcagacccttttagtcagaatggaaantctctagcagtggcgcccgaacagggaca

SEQ ID NO: 3 - DNA, 17 bp, Artificial Sequence
  sense primer for COXII
tgcccgccatcatccta

SEQ ID NO: 4 - DNA, 21 bp, Artificial Sequence
  probe for COXII
tcctcatcgccctcccatccc

SEQ ID NO: 5 - DNA, 23 bp, Artificial Sequence
  antisense primer for COXII
cgtctgttatgtaaaggatgcgt

SEQ ID NO: 6 - DNA, 17 bp, Artificial Sequence
  Sense primer for Exon 3 of the beta-actin gene
gcgcggctacagcttca

SEQ ID NO: 7 - DNA, 18 bp, Artificial Sequence
  Probe for Exon 3 of the beta-actin gene
caccacggccgagcggga

SEQ ID NO: 8 - DNA, 22 bp, Artificial Sequence
  Antisense primer for Exon 3 of the beta-actin gene
tctccttaatgtcacgcacgat

(19) United States
(12) Patent Application Publication
(10) Pub. No.: US 2012/0142627 A1
(43) Pub. Date: Jun. 7, 2012

(54) MONOPHOSPHATE PRODRUGS OF DAPD AND ANALOGS THEREOF

(75) Inventors: Raymond F. Schinazi, Atlanta, GA (US); Steven J. Coats, McDonough, GA (US)

(73) Assignees: Emory University, Atlanta, GA (US); RFS Pharma, LLC, Tucker, GA (US)

(21) Appl. No.: 13/306,967

(22) Filed: Nov. 29, 2011

Related U.S. Application Data
(60) Provisional application No. 61/420,293, filed on Dec. 6, 2010.

(51) Int. Cl. A61K 31/7072; A61P 31/20; A61P 31/18; C07F 9/6561; A61K 31/675

(52) U.S. Cl. 514/50; 544/244; 514/81

(57) ABSTRACT

The present invention is directed to compounds, compositions and methods for treating or preventing cancer and viral infections, in particular, HIV and HBV, in human patients or other animal hosts. The compounds are certain 6-substituted-2-amino-purine dioxolane monophosphates or phosphonates, and pharmaceutically acceptable, salts, prodrugs, and other derivatives thereof.

### FIELD OF THE INVENTION

The present invention is directed to compounds, methods and compositions for treating or preventing viral infections using nucleotide analogs. More specifically, the invention describes 6-substituted-2-amino purine dioxolane monophosphate and monophosphonate prodrugs and modified prodrug analogs, pharmaceutically acceptable salts, or other derivatives thereof, and the use thereof in the treatment of cancer or viral infection(s), in particular, human immunodeficiency virus (HIV-1 and HIV-2) and/or HBV. This invention teaches how to modify the metabolic pathway of specific 6-substituted-2-amino purine dioxolanes and deliver nucleotide triphosphates to reverse transcriptases and polymerases at heretofore unobtainable therapeutically-relevant concentrations.

### BACKGROUND OF THE INVENTION

Nucleoside analogs as a class have a well-established regulatory history, with more than 10 currently approved by the US Food and Drug Administration (US FDA) for treating human immunodeficiency virus (HIV), hepatitis B virus (HBV), or hepatitis C virus (HCV). The challenge in developing antiviral therapies is to inhibit viral replication without injuring the host cell. In HIV, a key target for drug development is reverse transcriptase (HIV-RT), a unique viral polymerase. This enzyme is active early in the viral replication cycle and converts the virus' genetic information from RNA into DNA, a process necessary for continued viral replication. Nucleoside reverse transcriptase inhibitors (NRTI) mimic natural nucleosides. In the triphosphate form, each NRTI competes with one of the four naturally occurring 2′-deoxynucleoside 5′-triphosphate (dNTP), namely, dCTP, dTTP, dATP, or dGTP for binding and DNA chain elongation near the active site of HIV-1 RT.

Reverse transcription is an essential event in the HIV-1 replication cycle and a major target for the development of antiretroviral drugs (see Parniak M A, Sluis-Cremer N. Inhibitors of HIV-1 reverse transcriptase. Adv. Pharmacol. 2000, 49, 67-109; Painter G R, Almond M R, Mao S, Liotta D C. Biochemical and mechanistic basis for the activity of nucleoside analogue inhibitors of HIV reverse transcriptase. Curr. Top. Med. Chem. 2004, 4, 1035-44; Sharma P L, Nurpeisov V, Hernandez-Santiago B, Beltran T, Schinazi R F. Nucleoside inhibitors of human immunodeficiency virus type 1 reverse transcriptase. Curr. Top. Med. Chem. 2004, 4 895-919). Two distinct groups of compounds have been identified that inhibit HIV-1 RT. These are the nucleoside or nucleotide RT inhibitors (NRTI) and the non-nucleoside RT inhibitors (NNRTI).

NRTI are analogs of deoxyribonucleosides that lack a 3′-OH group on the ribose sugar. They were the first drugs used to treat HIV-1 infection and they remain integral components of nearly all antiretroviral regimens.

In 1985, it was reported that the synthetic nucleoside 3′-azido-3′-deoxythymidine (zidovudine, AZT), one representative NRTI, inhibited the replication of HIV. Since then, several other NRTI, including but not limited to 2′,3′-dideoxyinosine (didanosine, ddI), 2′,3′-dideoxycytidine (zalcitabine, ddC), 2′,3′-dideoxy-2′,3′-didehydrothymidine (stavudine, d4T), (−)-2′,3′-dideoxy-3′-thiacytidine (lamivudine, 3TC), (−)-2′,3′-dideoxy-5-fluoro-3′-thiacytidine (emtricitabine, FTC), (1S,4R)-4-[2-amino-6-(cyclopropyl-amino)-9H-purin-9-yl]-2-cyclopentene-1-methanol succinate (abacavir, ABC), (R)-9-(2-phosphonylmethoxypropyl)adenine (PMPA, tenofovir disoproxil fumarate) (TDF), and (−)-carbocyclic 2′,3′-didehydro-2′,3′-dideoxyguanosine (carbovir) and its prodrug abacavir, have proven effective against HIV. After phosphorylation to the 5′-triphosphate by cellular kinases, these NRTI are incorporated into a growing strand of viral DNA causing chain termination, because they lack a 3′-hydroxyl group.

In general, to exhibit antiviral activity, NRTI must be metabolically converted by host-cell kinases to their corresponding triphosphate forms (NRTI-TP). The NRTI-TP inhibit HIV-1 RT DNA synthesis by acting as chain-terminators of DNA synthesis (see Goody R S, Muller B, Restle T. Factors contributing to the inhibition of HIV reverse transcriptase by chain terminating nucleotides in vitro and in vivo. FEBS Lett. 1991, 291, 1-5). Although combination therapies that contain one or more NRTI have profoundly reduced morbidity and mortality associated with AIDS, the approved NRTI can have significant limitations. These include acute and chronic toxicity, pharmacokinetic interactions with other antiretrovirals, and the selection of drug-resistant variants of HIV-1 that exhibit cross-resistance to other NRTI.

HIV-1 drug resistance within an individual arises from the genetic variability of the virus population and selection of resistant variants with therapy (see Chen R, Quinones-Mateu M E, Mansky L M. Drug resistance, virus fitness and HIV-1 mutagenesis. Curr. Pharm. Des. 2004, 10, 4065-70). HIV-1 genetic variability is due to the inability of HIV-1 RT to proofread nucleotide sequences during replication. This variability is increased by the high rate of HIV-1 replication, the accumulation of proviral variants during the course of HIV-1 infection, and genetic recombination when viruses of different sequence infect the same cell. As a result, innumerable genetically distinct variants (termed quasi-species) evolve within an individual in the years following initial infection. The development of drug resistance depends on the extent to which virus replication continues during drug therapy, the ease of acquisition of a particular mutation (or set of mutations), and the effect of drug resistance mutations on drug susceptibility and viral fitness. In general, NRTI therapy selects for viruses that have mutations in RT. Depending on the NRTI resistance mutation(s) selected, the mutant viruses typically exhibit decreased susceptibility to some or, in certain instances, all NRTI. From a clinical perspective, the development of drug resistant HIV-1 limits future treatment options by effectively decreasing the number of available drugs that retain potency against the resistant virus. This often requires more complicated drug regimens that involve intense dosing schedules and a greater risk of severe side effects due to drug toxicity. These factors often contribute to incomplete adherence to the drug regimen. Thus, the development of novel NRTI with excellent activity and safety profiles and limited or no cross-resistance with currently-available drugs is critical for effective therapy of HIV-1 infection.

The development of nucleoside analogs active against drug-resistant HIV-1 requires detailed understanding of the molecular mechanisms involved in resistance to this class of compounds. Accordingly, a brief overview of the mutations and molecular mechanisms of HIV-1 resistance to NRTI is provided. Two kinetically distinct molecular mechanisms of HIV-1 resistance to NRTI have been proposed (see Sluis-Cremer N, Arion D, Pamiak M A. Molecular mechanisms of HIV-1 resistance to nucleoside reverse transcriptase inhibitors (NRTIs). Cell Mol. Life. Sci. 2000; 57, 1408-22). One mechanism involves selective decreases in NRTI-TP versus normal dNTP incorporation during viral DNA synthesis. This resistance mechanism has been termed discrimination. The second mechanism involves selective removal of the chain-terminating NRTI-monophosphate (NRTI-MP) from the prematurely terminated DNA chain (see Arion D, Kaushik N, McCormick S, Borkow G, Parniak M A. Phenotypic mechanism of HIV-1 resistance to 3′-azido-3′-deoxythymidine (AZT): increased polymerization processivity and enhanced sensitivity to pyrophosphate of the mutant viral reverse transcriptase. Biochemistry. 1998, 37, 15908-17; Meyer P R, Matsuura S E, Mian A M, So A G, Scott W A. A mechanism of AZT resistance: an increase in nucleotide-dependent primer unblocking by mutant HIV-1 reverse transcriptase. Mol. Cell. 1999, 4, 35-43). This mechanism has been termed excision.

The discrimination mechanism involves the acquisition of one or more resistance mutations in RT that improve the enzyme's ability to discriminate between the natural dNTP substrate and the NRTI-TP. In this regard, resistance is typically associated with a decreased catalytic efficiency of NRTI-TP incorporation. NRTI-TP (and dNTP) catalytic efficiency is driven by two kinetic parameters, (i) the affinity of the nucleotide for the RT polymerase active site (K_{d}) and (ii) the maximum rate of nucleotide incorporation (kpol), both of which can be determined using pre-steady-state kinetic analyses (see Kati W M, Johnson K A, Jerva L F, Anderson K S. Mechanism and fidelity of HIV reverse transcriptase. J. Biol. Chem. 1992, 26: 25988-97).

For the excision mechanism of NRTI resistance, the mutant HIV-1 RT does not discriminate between the natural dNTP substrate and the NRTI-TP at the nucleotide incorporation step (see Kerr S G, Anderson K S. Pre-steady-state kinetic characterization of wild type and 3′-azido-3′-deoxythymidine (AZT) resistant human immunodeficiency virus type 1 reverse transcriptase: implication of RNA directed DNA polymerization in the mechanism of AZT resistance. Biochemistry. 1997, 36, 14064-70). Instead, RT containing “excision” mutations shows an increased capacity to unblock NRTI-MP terminated primers in the presence of physiological concentrations of ATP (typically within the range of 0.8-4 mM) or pyrophosphate (PPi) (see Arion D, Kaushik N, McCormick S, Borkow G, Parniak M A. Phenotypic mechanism of HIV-1 resistance to 3′-azido-3′-deoxythymidine (AZT): increased polymerization processivity and enhanced sensitivity to pyrophosphate of the mutant viral reverse transcriptase. Biochemistry. 1998, 37, 15908-17; Meyer P R, Matsuura S E, Mian A M, So A G, Scott W A. A mechanism of AZT resistance: an increase in nucleotide-dependent primer unblocking by mutant HIV-1 reverse transcriptase. Mol. Cell. 1999, 4, 35-43). NRTI resistance mutations associated with the excision mechanism include thymidine analog mutations (TAMS) and T69S insertion mutations.

Another virus that causes a serious human health problem is the hepatitis B virus (HBV). HBV is second only to tobacco as a cause of human cancer. The mechanism by which HBV induces cancer is unknown. It is postulated that it may directly trigger tumor development, or indirectly trigger tumor development through chronic inflammation, cirrhosis, and cell regeneration associated with the infection.

After a 2- to 6-month incubation period, during which the host is typically unaware of the infection, HBV infection can lead to acute hepatitis and liver damage, resulting in abdominal pain, jaundice and elevated blood levels of certain enzymes. HBV can cause fulminant hepatitis, a rapidly progressive, often fatal form of the disease in which large sections of the liver are destroyed.

Patients typically recover from the acute phase of HBV infection. In some patients, however, the virus continues replication for an extended or indefinite period, causing a chronic infection. Chronic infections can lead to chronic persistent hepatitis. Patients infected with chronic persistent HBV are most common in developing countries. By mid-1991, there were approximately 225 million chronic carriers of HBV in Asia alone and worldwide almost 300 million carriers. Chronic persistent hepatitis can cause fatigue, cirrhosis of the liver, and hepatocellular carcinoma, a primary liver cancer.

In industrialized countries, the high-risk group for HBV infection includes those in contact with HBV carriers or their blood samples. The epidemiology of HBV is very similar to that of HIV/AIDS, which is a reason why HBV infection is common among patients infected with HIV or suffering from AIDS. However, HBV is more contagious than HIV.

3TC (lamivudine), interferon alpha-2b, peginterferon alpha-2a, hepsera (adefovir dipivoxil), baraclude (entecavir), and Tyzeka (Telbivudine) are currently FDA-approved drugs for treating HBV infection. However, some of the drugs have severe side effects, and viral resistance develops rapidly in patients treated with these drugs.

Proliferative disorders are one of the major life-threatening diseases and have been intensively investigated for decades. Cancer now is the second leading cause of death in the United States, and over 500,000 people die annually from this proliferative disorder. A tumor is an unregulated, disorganized proliferation of cell growth. A tumor is malignant, or cancerous, if it has the properties of invasiveness and metastasis. Invasiveness refers to the tendency of a tumor to enter surrounding tissue, breaking through the basal laminas that define the boundaries of the tissues, thereby often entering the body's circulatory system. Metastasis refers to the tendency of a tumor to migrate to other areas of the body and establish areas of proliferation away from the site of initial appearance.

Cancer is not fully understood on the molecular level. It is known that exposure of a cell to a carcinogen such as certain viruses, certain chemicals, or radiation, leads to DNA alteration that inactivates a “suppressive” gene or activates an “oncogene.” Suppressive genes are growth regulatory genes, which upon mutation, can no longer control cell growth. Oncogenes are initially normal genes (called prooncongenes) that by mutation or altered context of expression become transforming genes. The products of transforming genes cause inappropriate cell growth. More than twenty different normal cellular genes can become oncogenes by genetic alteration. Transformed cells differ from normal cells in many ways, including cell morphology, cell-to-cell interactions, membrane content, cytoskeletal structure, protein secretion, gene expression and mortality (transformed cells can grow indefinitely).

All of the various cell types of the body can be transformed into benign or malignant tumor cells. The most frequent tumor site is lung, followed by colorectal, breast, prostate, bladder, pancreas and then ovary. Other prevalent types of cancer include leukemia, central nervous system cancers, including brain cancer, melanoma, lymphoma, erythroleukemia, uterine cancer, and head and neck cancer.

Cancer is now primarily treated with one or a combination of three means of therapies: surgery, radiation and chemotherapy. Surgery involves the bulk removal of diseased tissue. While surgery is sometimes effective in removing tumors located at certain sites, for example, in the breast, colon and skin, it cannot be used in the treatment of tumors located in other areas, such as the backbone, or in the treatment of disseminated neoplastic conditions such as leukemia.

Chemotherapy involves the disruption of cell replication or cell metabolism. It is used most often in the treatment of leukemia, as well as breast, lung, and testicular cancer. There are five major classes of chemotherapeutic agents currently in use for the treatment of cancer: natural products and their derivatives; anthacyclines; alkylating agents; antiproliferatives (also called antimetabolites); and hormonal agents. Chemotherapeutic agents are often referred to as antineoplastic agents.

Several synthetic nucleosides, such as 5-fluorouracil, have been identified that exhibit anticancer activity. 5-Fluorouracil has been used clinically in the treatment of malignant tumors, including, for example, carcinomas, sarcomas, skin cancer, cancer of the digestive organs, and breast cancer. 5-Fluorouracil, however, causes serious adverse reactions such as nausea, alopecia, diarrhea, stomatitis, leukocytic thrombocytopenia, anorexia, pigmentation and edema.

In light of the fact that acquired immune deficiency syndrome, AIDS-related complex, cancer, and HBV have reached alarming levels worldwide, and have significant and in some cases tragic effects on the effected patient, there remains a strong need to provide new effective pharmaceutical agents to treat these diseases, with agents that have low toxicity to the host.

It would be advantageous to provide new antiviral or chemotherapy agents, compositions including these agents, and methods of treatment using these agents, particularly to treat drug resistant cancers or mutant viruses. The present invention provides such agents, compositions and methods.

### SUMMARY OF THE INVENTION

The present invention provides compounds, methods and compositions for treating or preventing cancer, an HIV-1 or HIV-2 infection, and/or HBV infection in a host. The methods involve administering a therapeutically or prophylactically-effective amount of at least one compound as described herein to treat or prevent an infection by, or an amount sufficient to reduce the biological activity of, cancer or an HIV-1, HIV-2, or HBV infection. The pharmaceutical compositions include one or more of the compounds described herein, in combination with a pharmaceutically acceptable carrier or excipient, for treating a host with cancer or infected with HIV-1, HIV-2, or HBV. The formulations can further include at least one additional therapeutic agent, which in one embodiment is AZT or 3TC. In addition, the present invention includes processes for preparing such compounds.

The compounds are monophosphate or monophosphonate forms of various 6-substituted-2-amino purine dioxolanes, or analogs of the monophosphate forms, which also become triphosphorylated when administered in vivo. By preparing the monophosphate prodrugs, we have developed a method for delivering nucleotide triphosphates to the polymerase or reverse transcriptase, which before this invention was not possible, or at least not possible at therapeutically-relevant concentrations. This invention allows for a new and novel series of nucleotide triphosphates to be prepared in vivo and enlisted as antiviral agents or anticancer agents.

The compounds described herein include monophosphate, phosphonate, and other analogs of β-D-6-substituted-2-amino purine dioxolanes.

In one embodiment, the active compound is of one of the following formulas:

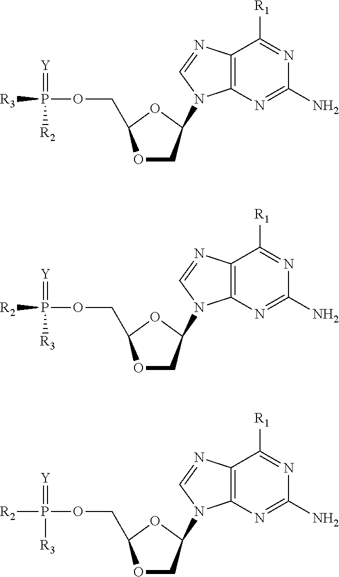

or a pharmaceutically acceptable salt or prodrug thereof, wherein:
i) R¹ is an atom or group removed in vivo to form OH when administered as the parent nucleoside, for example, halogen (F, Cl, Br, I), OR′, N(R′)₂, SR′, OCOR′, NHCOR′, N(COR′)COR′, SCOR′, OCOOR′, and NHCOOR′.
   each R′ is independently H, a lower alkyl (C₁-C₆), lower haloalkyl (C₁-C₆), lower alkoxy (C₁-C₆), lower alkenyl (C₂-C₆), lower alkynyl (C₂-C₆), lower cycloalkyl (C₃-C₆) aryl, heteroaryl, alkylaryl, or arylalkyl, wherein the groups can be substituted with one or more substituents as defined above, for example, hydroxyalkyl, aminoalkyl, and alkoxyalkyl.
Y is O or S;
R² and R³, when administered in vivo, are ideally capable of providing the nucleoside monophosphate monophosphonate, thiomonophosphonate, or thiomonophosphate. Representative R² and R³ are independently selected from:
   (a) OR⁸ where R⁸ is H, C₁₋₂₀ alkyl, C₃₋₆ cycloalkyl, C₁₋₆ haloalkyl, aryl, or heteroaryl which includes, but is not limited to, phenyl or naphthyl optionally substituted with one to three substituents independently selected from the group consisting of C₁₋₆ alkyl, C₂₋₆ alkenyl, C₂₋₆ alkynyl, C₁₋₆ alkoxy, (CH₂)₁₋₆CO₂R^{9a}, halogen, C₁₋₆ haloalkyl, —N(R^{9a})₂, C₁₋₆ acylamino, —NHSO₂C₁₋₆ alkyl, —SO₂N(R^{9a})₂, —SO₂C₁₋₆ alkyl, COR^{9b}, nitro and cyano;
   R^{9a} is independently H or C₁₋₆ alkyl;
   R^{9b} is —OR^{9a} or —N(R^{9a})₂;
(b)

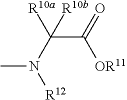

where R^{10a} and R^{10b} are:
(i) independently selected from the group consisting of H, C₁₋₁₀ alkyl, —(CH₂)ᵣNR^{9a}₂, C₁₋₆ hydroxyalkyl, —CH₂SH, —(CH₂)₂S(O)ₚMe, —(CH₂)₃NHC(═NH)NH₂, (1H-indol-3-yl)methyl, (1H-imidazol-4-yl)methyl, —(CH₂)ₘCOR^{9b}, aryl and aryl-C₁₋₃ alkyl, said aryl groups optionally substituted with a group selected from the group consisting of hydroxyl, C₁₋₁₀ alkyl, C₁₋₆ alkoxy, halogen, nitro, and cyano;
(ii) R^{10a} is H and R^{10b} and R¹² together are (CH₂)₂₋₄ to form a ring that includes the adjoining N and C atoms;
(iii) R^{10a} and R^{10b} together are (CH₂)ₙ to form a ring;
(iv) R^{10a} and R^{10b} both are C₁₋₆ alkyl; or
(v) R^{10a} is H and R^{10b} is H, CH₃, CH₂CH₃, CH(CH₃)₂, CH₂CH(CH₃)₂, CH(CH₃)CH₂CH₃, CH₂Ph, CH₂-indol-3-yl, —CH₂CH₂SCH₃, CH₂CO₂H, CH₂C(O)NH₂, CH₂CH₂COOH, CH₂CH₂C(O)NH₂, CH₂CH₂CH₂CH₂NH₂—CH₂CH₂CH₂NHC(NH)NH₂, CH₂-imidazol-4-yl, CH₂OH, CH(OH)CH₃, CH₂((4′-OH)-Ph), CH₂SH, or lower cycloalkyl;
p is 0 to 2;
r is 1 to 6;
n is 4 or 5;
m is 0 to 3;
   R¹¹ is H, C₁₋₁₀ alkyl, or C₁₋₁₀ alkyl substituted with a lower alkyl, alkoxy, di(lower alkyl)-amino, fluoro, C₃₋₁₀ cycloalkyl, cycloalkyl alkyl, cycloheteroalkyl, aryl, such as phenyl, heteroaryl, such as, pyridinyl, substituted aryl, or substituted heteroaryl; wherein the substituents are C₁₋₅ alkyl, or C₁₋₅ alkyl substituted with a lower alkyl, alkoxy, di(lower alkyl)-amino, fluoro, C₃₋₁₀ cycloalkyl, or cycloalkyl;
   R¹² is H, C₁₋₃ alkyl, or R^{10a}, or R^{10b} and R¹² together are (CH₂)₂₋₄ so as to form a ring that includes the adjoining N and C atoms;
(c) an O attached lipid (including a phospholipid), an N or O attached peptide, an O attached cholesterol, or an O attached phytosterol;
(d) R² and R³ may come together to form a ring

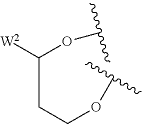

where W² is selected from a group consisting of phenyl or monocyclic heteroaryl, optionally substituted with one to three substituents independently selected from the group consisting of C₁₋₆ alkyl, CF₃, C₂₋₆ alkenyl, C₁₋₆ alkoxy, OR^{9c}, CO₂R^{9a}, COR^{9a}, halogen, C₁₋₆ haloalkyl, —N(R^{9a})₂, C₁₋₆ acylamino, CO₂N(R^{9a})₂, SR^{9a}, —NHSO₂C₁₋₆ alkyl, —SO₂N(R^{9a})₂, —SO₂C₁₋₆ alkyl, COR^{9b}, and cyano, and wherein said monocyclic heteroaryl and substituted monocyclic heteroaryl has 1-2 heteroatoms that are independently selected from the group consisting of N, O, and S with the provisos that:
a) when there are two heteroatoms and one is O, then the other can not be O or S, and
b) when there are two heteroatoms and one is S, then the other can not be O or S;
R^{9a} is independently H or C₁₋₆ alkyl;
R^{9b} is —OR^{9a} or —N(R^{9a})₂;
R^{9c} is H or C₁₋₆ acyl;
(e)

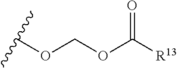

where R¹³ is selected from a group consisting of H, C₁₋₁₀ alkyl, C₁₋₁₀ alkyl optionally substituted with a lower alkyl, alkoxy, di(lower alkyl)-amino, fluoro, C₃₋₁₀ cycloalkyl, cycloalkyl alkyl, cycloheteroalkyl, aryl, such as phenyl, heteroaryl, such as, pyridinyl, substituted aryl, or substituted heteroaryl; wherein the substituents are C₁₋₅ alkyl, or C₁₋₅ alkyl substituted with a lower alkyl, alkoxy, di(lower alkyl)-amino, fluoro, C₃₋₁₀ cycloalkyl, or cycloalkyl;
f) R² and R³ may come together to form a ring

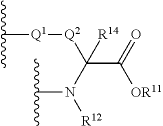

where R¹⁴ is: (i) independently selected from the group consisting of H, C₁₋₁₀ alkyl, —(CH₂)ᵣNR₂^{9a}, C₁₋₆ hydroxyalkyl, —CH₂SH, —(CH₂)₂S(O)ₚMe, —(CH₂)₃NHC(═NH)NH₂, (1H-indol-3-yl)methyl, (1H-imidazol-4-yl)methyl, —(CH₂)ₘCOR^{9b}, aryl and aryl-C₁₋₃ alkyl or heteroaryl and heteroaryl-C₁₋₃ alkyl, said aryl and heteroaryl groups optionally substituted with a group selected from the group consisting of hydroxyl, C₁₋₁₀ alkyl, C₁₋₆ alkoxy, halogen, nitro, and cyano; (ii) R¹⁴ is H, CH₃, CH₂CH₃, CH(CH₃)₂, CH₂CH(CH₃)₂, CH(CH₃)CH₂CH₃, CH₂Ph, CH₂-indol-3-yl, —CH₂CH₂SCH₃, CH₂CO₂H, CH₂C(O)NH₂, CH₂CH₂COOH, CH₂CH₂C(O)NH₂, CH₂CH₂CH₂CH₂NH₂, CH₂CH₂CH₂NHC(NH)NH₂, CH₂-imidazol-4-yl, CH₂OH, CH(OH)CH₃, CH₂((4′-OH)-Ph), CH₂SH, or lower cycloalkyl;
is 0 to 2;
r is 1 to 6;
m is 0 to 3
Q¹ is NR^{9a}, O, or S
Q² is C₁₋₁₀ alkyl, C₁₋₆ hydroxyalkyl, aryl and aryl-C₁₋₃ alkyl, heteroaryl and heteroaryl-C₁₋₃ alkyl, said aryl and heteroaryl groups optionally substituted with a group selected from the group consisting of hydroxyl, C₁₋₁₀ alkyl, C₁₋₆ alkoxy, fluoro, and chloro;
   R¹¹ is H, C₁₋₁₀ alkyl, C₁₋₁₀ alkyl optionally substituted with a lower alkyl, alkoxy, di(lower alkyl)-amino, fluoro, C₃₋₁₀ cycloalkyl, cycloalkyl alkyl, cycloheteroalkyl, aryl, such as phenyl, heteroaryl, such as, pyridinyl, substituted aryl, or substituted heteroaryl; wherein the substituents are C₁₋₅ alkyl, or C₁₋₅ alkyl substituted with a lower alkyl, alkoxy, di(lower alkyl)-amino, fluoro, C₃₋₁₀ cycloalkyl, or cycloalkyl;
   R¹² is H, or C₁₋₃ alkyl, or R^{14b}) and R¹² together are (CH₂)₂ so as to form a ring that includes the adjoining N and C atoms;

In one embodiment, the compounds have one of the following formulas:

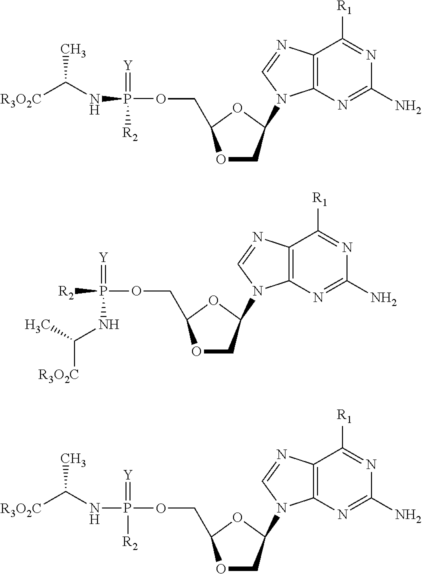

The first two compounds show both enantiomers of the phosphorus atom. Coupled with the chiral carbon on the dioxolane, these compounds exist as diastereomers. The third compound shows a racemic phosphorus, and a chiral dioxolane. In all three compounds, the amino acid attached to the phosphorus is L-alanine, or an ester derivative thereof.

In one embodiment, R₁ is selected from the group consisting of halo (i.e., Cl, Br, I, and F), NH₂, OMe, and NH—C₃₋₆ cycloalkyl. In another embodiment, R₁ is selected from the group consisting of Cl, NH₂, OMe, and NH—C₃ cycloalkyl.

The prodrug compounds described herein are in the form of the β-D-configuration, or at least the β-D-configuration is the major configuration, with an enantiomeric excess greater than 95%, preferably greater than 97%. Where one or R₂ and R₃ is alanine or an alanine ester, with the nitrogen attached to the phosphorus, the alanine is preferably in the L configuration.

The prodrug compounds can be prepared, for example, by preparing the 5′-OH analogs, then converting these to the mono-phosphates, phosphonate, or other analogs.

The compounds described herein are inhibitors of HIV-1, HIV-2, cancer, and/or HBV. Therefore, these compounds can also be used to treat patients that are co-infected with two or more of HIV-1, HIV-2, cancer, and/or HBV.

The prodrug can have a chiral carbon or phosphorus atom on the moiety attached to the 5′-OH. Where the phosphorus atom on the side chain is chiral, it can exist in R or S form.

In one embodiment, the drug combinations include a) the DAPD and DAPD analog prodrugs described herein, and b) zidovudine (AZT) or other thymidine nucleoside antiretroviral agents. AZT is effective against HIV containing the K65R mutation, and DXG, the active metabolite of the DAPD and DAPD analog prodrugs described herein, can select for the K65R mutation. By co-administering AZT, the population of virus developing the K65 mutation can be controlled. In one aspect of this embodiment, the dosage of AZT or other thymidine nucleoside antiretroviral agents is lower than conventional dosages, in order to reduce side effects, while still maintaining an efficacious therapeutic level of the therapeutic agent. For example, to minimize side effects associated with administration of AZT, such as bone marrow toxicity resulting in anemia, one can effectively lower the dosage to somewhere between around 100 and around 250 mg bid, preferably around 200 mg bid.

Using the lower (but still effective) dosage of AZT, one can minimize bone marrow toxicity believed to be secondary to zidovudine-monophosphate (AZT-MP) accumulation by significantly lowering the amount of AZT-MP present in the patient, without significant changes in the levels of zidovudine-triphosphate (AZT-TP), responsible for antiviral activity.

In another aspect of this embodiment, the therapeutic combinations further include at least one additional agent selected from non-nucleoside reverse transcriptase inhibitors (“NNRTI”), polymerase inhibitors, protease inhibitors, fusion inhibitors, entry inhibitors, attachment inhibitors, and integrase inhibitors, such as raltegravir (Isentress) or MK-0518, GS-9137 (elvitegravir, Gilead Sciences), GS-8374 (Gilead Sciences), or GSK-364735.

In any of these embodiments, additional therapeutic agents can be used in combination with these agents, particularly including agents with a different mode of attack. Such agents include but are not limited to: antivirals, such as cytokines, e.g., rIFN alpha, rIFN beta, rIFN gamma; amphotericin B as a lipid-binding molecule with anti-HIV activity; a specific viral mutagenic agent (e.g., ribavirin), an HIV VIF inhibitor, and an inhibitor of glycoprotein processing.

In any of these embodiments, the various individual therapeutic agents, such as the zidovudine (ZDV, AZT) or other thymidine nucleoside antiretroviral agent and non-thymidine nucleoside antiretroviral agents that select for the K65R mutation in the first embodiment, can be administered in combination or in alternation. When administered in combination, the agents can be administered in a single or in multiple dosage forms. In some embodiments, some of the antiviral agents are orally administered, whereas other antiviral agents are administered by injection, which can occur at around the same time, or at different times.

The invention encompasses combinations of the two types of antiviral agents, or pharmaceutically acceptable derivatives thereof, that are synergistic, i.e., better than either agent or therapy alone.

The antiviral combinations described herein provide means of treatment which can not only reduce the effective dose of the individual drugs required for antiviral activity, thereby reducing toxicity, but can also improve their absolute antiviral effect, as a result of attacking the virus through multiple mechanisms. That is, the combinations are useful because their synergistic actions permit the use of less drug, increase the efficacy of the drugs when used together in the same amount as when used alone. Similarly, the novel antiviral combinations provide a means for circumventing the development of viral resistance to a single therapy, thereby providing the clinician with a more efficacious treatment.

The disclosed combination or alternation therapies are useful in the prevention and treatment of HIV infections and other related conditions such as AIDS-related complex (ARC), persistent generalized lymphadenopathy (PGL), AIDS-related neurological conditions, anti-HIV antibody positive and HIV-positive conditions, Kaposi's sarcoma, thrombocytopenia purpurea and opportunistic infections. In addition, these compounds or formulations can be used prophylactically to prevent or retard the progression of clinical illness in individuals who are anti-HIV antibody or HIV-antigen positive or who have been exposed to HIV. For example, the compositions can prevent or retard the development of K65R resistant HIV. The therapy can be also used to treat other viral infections, such as HIV-2.

The compounds can be prepared, for example, by preparing the 5′-OH analogs, then converting these to the mono-phosphates, phosphonate, or other analogs. If an enantiomerically-enriched phosphorus atom is desired, and the prodrug does not include a chiral carbon (so as to form a diastereomer), one can perform an additional step of enantiomerically enriching the prodrug, for example, by using enzymatic digestion. That is, certain enzymes, such as Rp-specific snake venom phosphodiesterase (svPDE) and Sp-specific nuclease P1 can be used to prepare the desired enantiomer, by digesting the undesired enantiomer. Chiral chromatography can also be used to prepare individual chiral phosphorus compounds.

In one embodiment, the invention relates to a process for preparing the dioxolane compounds described herein. The process first involves preparing compounds of the general formula (1)

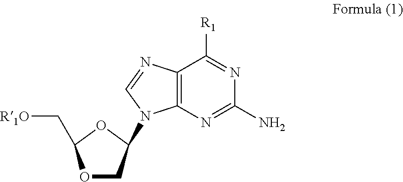

and pharmaceutically acceptable salts or prodrug thereof; wherein, R′₁ is a hydroxyl protecting group; and R₁ is as defined above,

by reacting a compound of the general formula (2)

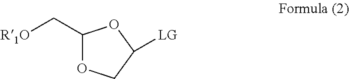

wherein LG is a leaving group as defined according to J. March, “Advanced Organic Chemistry”, 3rd edition, Wiley 1985,

with a 2,6-substituted purine derivative of the general formula (5)

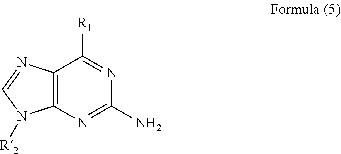

wherein; R′₂ is a silyl radical,

in the presence of a Lewis acid, solvent, and additionally in the presence of a 2-cyanoethanoate compound or a silylated derivative of a 2-cyanoethanoate compound.

After this step is completed, the hydroxyl protecting group R′₁ is removed, and the hydroxyl group is coupled to a phosphate or phosphonate group, or derivative thereof. The coupling step generally involves formation of a phosphate ester, wherein an activated phosphorous compound (i.e., containing a P—Cl bond, or other suitable bond with a leaving group) is reacted with the OH group to form HCl and the P—O linkage, or other suitable “H-leaving group” and the P—O linkage.

A representative phosphorous-containing reagent to couple with the —OH group is shown below:

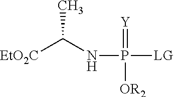

where R₂ is selected from the group consisting of C₁₋₈ alkyl, aryl, and heteroaryl, wherein the alkyl, aryl, and heteroaryl moieties can optionally substituted with from one to three substituents as described elsewhere herein as suitable substituents for such moieties;

LG is a leaving group, such as a halo (i.e., I, Br, Cl, or F), tosylate, brosylate, nosylate, mesylate, triflate, and the like, and

Y is O or S.

In one embodiment, the compound disclosed above includes a chiral phosphorus atom in enantiomerically-enriched form, so that the resulting prodrug also includes a chiral phosphorus atom in enantiomerically-enriched form.

A representative coupling reaction is shown below:

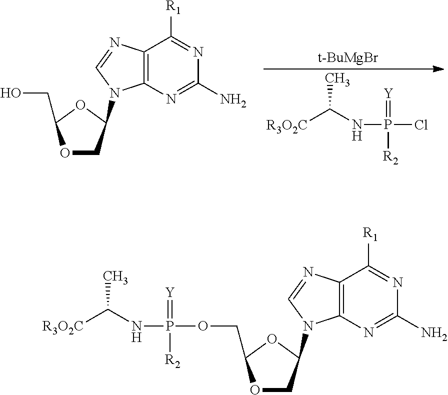

The process of the invention can be used to produce racemic prodrug compounds, or optically pure or enriched prodrug compounds, through choice of precursors having an appropriate optical configuration. If the phosphorus atom in the precursor used to prepare the phosphate or phosphonate prodrug is chiral, then appropriate diastereomers can be produced.

The hydroxyl protecting group R′₁ can be selected from all alcohol protecting group known and suitable to one skilled in the art. For example, alcohols protecting groups as described in “T. W. Greene, P. G. M. Wuts, “Protective Groups in Organic Synthesis”, 3^{rd} edition, Wiley 1999, pp. 17-200.

Leaving groups (“LG”) are preferably selected from iodine, bromine, C₁₋₂₀ acyloxy radical, C₁₋₂₀ alkylsulfonyloxy radical, C₁₋₂₀ arylsulfonyloxy radical, C₁₋₂₀ alkoxy radical and C₁₋₂₀ aryloxy radical.

The 2,6-disubstituted purine derivative of the general formula (5) contains at least one C₁₋₂₀ silyl radical R′₄, and optionally further silyl radicals on functions in positions 2 and 6, when possible, to act as amino protective groups.

The alpha cyano carbonyl compound used is a 2-cyanoethanoate ester, a 2-cyano ketone or a 2-cyanoethanoic acid derivative having 5 to 20 C atoms of the general formula (3)

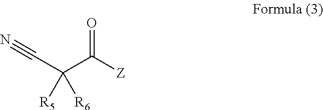

wherein Z may be hydrogen, an alkyl radical having from 1 to 20 C atoms, an aryl radical having from 6 to 20 C atoms or an alkyloxy group having from 1 to 20 C atoms and R₅ and R₆ can be, independently, a hydrogen, an acyl radical of an aromatic or aliphatic carboxylic acid having from 2 to 20 C atoms, an alkyl radical having from 1 to 20 C atoms or an aryl radical having from 6 to 20 C atoms.

The silylated derivative of 2-cyanoethanoate ester compound used is a silyl derivative of a 2-cyanoethanoate ester, of a 2-cyano ketone or of a 2-cyanoethanoic acid derivative of the general formula (4)

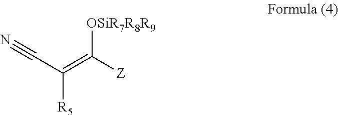

wherein Z and R₅ are as described above, and R₇, R₈ and R₉ may be independently of one another an aliphatic or aromatic radical having from 1 to 20 C atoms.

In general all aprotic organic solvents can be used for the process. The reaction is preferably carried out under atmospheric pressure at a temperature between −25° C. and the boiling point of the solvent.

The present invention also provides a recrystallization process for purifying compounds of the general formula (I) obtained by the process of the invention.

Preferred methods for removing OH protective acyl radical groups are reaction with ammonia, aliphatic amines, basic aqueous hydrolysis, or reaction with alcoholates. Preferred methods for forming the prodrugs described herein include reacting the resulting OH group with a phosphorus compound that includes a leaving group, such as chloride, that can be displaced by the OH group to form a P—O bond.

### BRIEF DESCRIPTION OF THE FIGURES

FIG. 1 is a chart showing the synthesis of certain of the prodrugs described herein, showing the relation between the compound identifiers and the various R groups in the reactants and products.

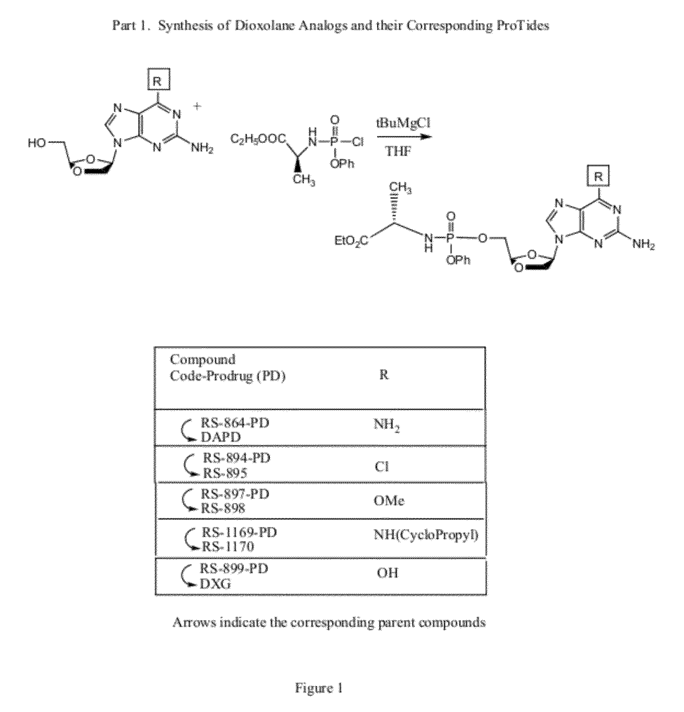

FIG. 2 is a chart showing the cell types against which the cytotoxicity of the prodrugs was measured, and highlighting how the cytotoxicity was measured.

FIG. 3 is a chart showing how the prodrugs were evaluated for anti-HIV activity in human PBM cells.

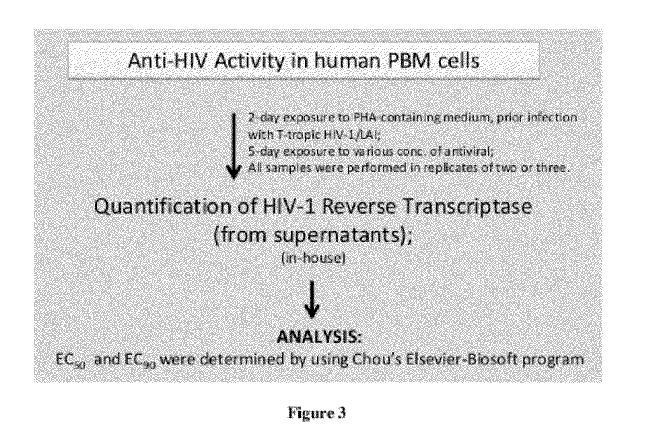

FIG. 4 is a chart showing how the prodrugs were evaluated for anti-HBV activity in the HBV AD38 system.

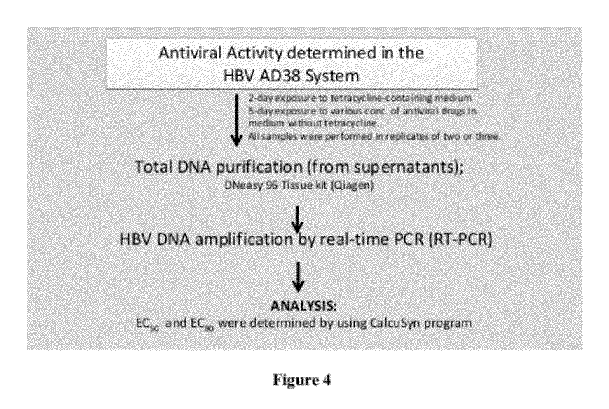

FIG. 5 is a chart showing how the cellular pharmacology of the prodrugs was measured, and, in particular, how intracellular NTP (nucleotide triphosphate) levels were measured.

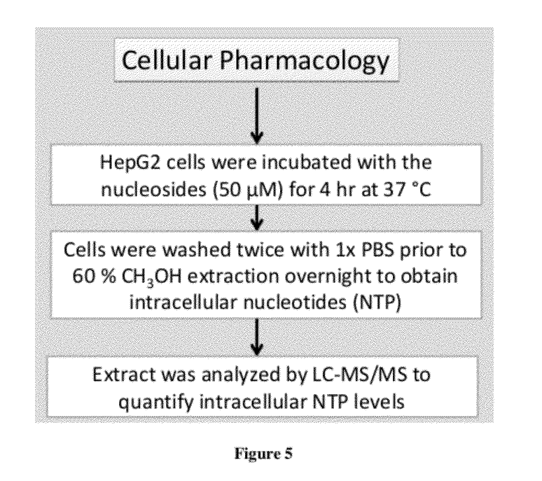

FIG. 6 is chart showing the intracellular concentration of DXG-TP (DXG triphosphate) in PBM cells incubated with the identified compounds for 4 h at 50 mM. The data plotted represent the mean value and S.D. of experiments with PBM cells, shown in terms of pmol per 10⁶ cells.

FIG. 7 is a chart showing the intracellular levels of DXG-TP in HepG2 cells (pmol per 10⁶ cells) and antiviral activity against HIV (blue) and HBV (black).

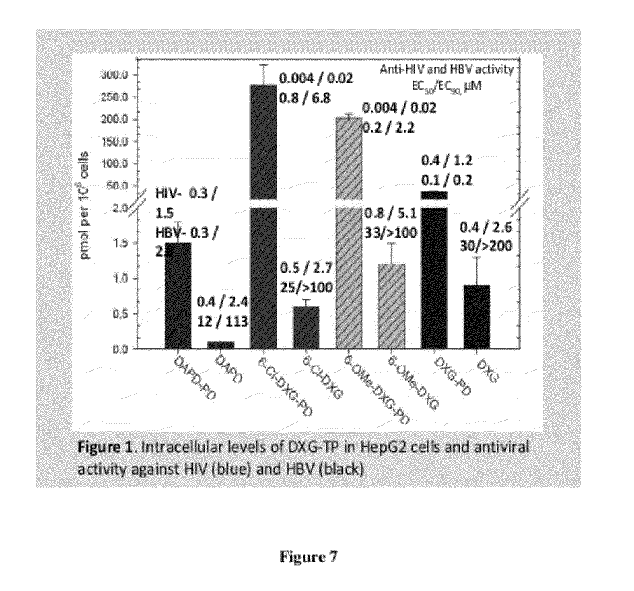

FIG. 8 is a chart showing the intracellular levels of dioxolane nucleoside-triphosphate levels in HepG2 cells (pmol per 10⁶ cells).

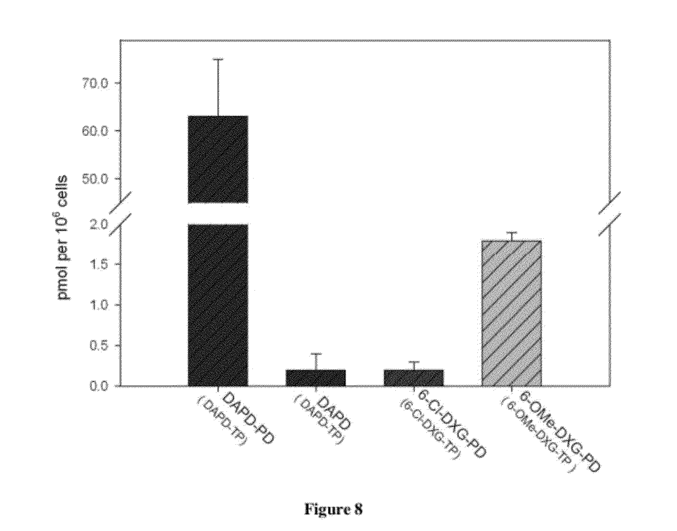

### DETAILED DESCRIPTION

The present invention provides novel and potent nucleosides with modifications at the C6 position of the purine ring of DAPD, which show increased cellular penetration and improves in vitro potency against HIV and HBV, relative to DAPD.

The 6-substituted-2-amino purine dioxolane monophosphate prodrugs described herein show inhibitory activity against HIV, cancer, and HBV. Therefore, the compounds can be used to treat or prevent a viral infection in a host, or reduce the biological activity of the virus. The host can be a mammal, and in particular, a human, infected with HIV-1, HIV-2, cancer, and/or HBV. The methods involve administering an effective amount of one or more of the 6-substituted-2-amino purine dioxolanes monophosphate prodrugs described herein.

Pharmaceutical formulations including one or more compounds described herein, in combination with a pharmaceutically acceptable carrier or excipient, are also disclosed. In one embodiment, the formulations include at least one compound described herein and at least one further therapeutic agent.

The present invention will be better understood with reference to the following definitions:

### I. DEFINITIONS

The term “independently” is used herein to indicate that the variable, which is independently applied, varies independently from application to application. Thus, in a compound such as R″XYR″, wherein R″ is “independently carbon or nitrogen,” both R″ can be carbon, both R″ can be nitrogen, or one R″ can be carbon and the other R″ nitrogen.

As used herein, the term “enantiomerically pure” refers to a nucleotide composition that comprises at least approximately 95%, and, preferably, approximately 97%, 98%, 99% or 100% of a single enantiomer of that nucleotide.

As used herein, the term “substantially free of” or “substantially in the absence of” refers to a nucleotide composition that includes at least 85 to 90% by weight, preferably 95% to 98% by weight, and, even more preferably, 99% to 100% by weight, of the designated enantiomer of that nucleotide. In a preferred embodiment, the compounds described herein are substantially free of enantiomers.

Similarly, the term “isolated” refers to a nucleotide composition that includes at least 85 to 90% by weight, preferably 95% to 98% by weight, and, even more preferably, 99% to 100% by weight, of the nucleotide, the remainder comprising other chemical species or enantiomers.

The term “alkyl,” as used herein, unless otherwise specified, refers to a saturated straight, branched, or cyclic, primary, secondary, or tertiary hydrocarbons, including both substituted and unsubstituted alkyl groups. The alkyl group can be optionally substituted with any moiety that does not otherwise interfere with the reaction or that provides an improvement in the process, including but not limited to but limited to halo, haloalkyl, hydroxyl, carboxyl, acyl, aryl, acyloxy, amino, amido, carboxyl derivatives, alkylamino, dialkylamino, arylamino, alkoxy, aryloxy, nitro, cyano, sulfonic acid, thiol, imine, sulfonyl, sulfanyl, sulfinyl, sulfamonyl, ester, carboxylic acid, amide, phosphonyl, phosphinyl, phosphoryl, phosphine, thioester, thioether, acid halide, anhydride, oxime, hydrozine, carbamate, phosphonic acid, phosphonate, either unprotected, or protected as necessary, as known to those skilled in the art, for example, as taught in Greene, et al., Protective Groups in Organic Synthesis, John Wiley and Sons, Second Edition, 1991, hereby incorporated by reference. Specifically included are CF₃ and CH₂CF₃

In the text, whenever the term C(alkyl range) is used, the term independently includes each member of that class as if specifically and separately set out. The term “alkyl” includes C₁₋₂₂ alkyl moieties, and the term “lower alkyl” includes C₁₋₆ alkyl moieties. It is understood to those of ordinary skill in the art that the relevant alkyl radical is named by replacing the suffix “-ane” with the suffix “-yl”.

The term “alkenyl” refers to an unsaturated, hydrocarbon radical, linear or branched, in so much as it contains one or more double bonds. The alkenyl group disclosed herein can be optionally substituted with any moiety that does not adversely affect the reaction process, including but not limited to but not limited to those described for substituents on alkyl moieties. Non-limiting examples of alkenyl groups include ethylene, methylethylene, isopropylidene, 1,2-ethane-diyl, 1,1-ethane-diyl, 1,3-propane-diyl, 1,2-propane-diyl, 1,3-butane-diyl, and 1,4-butane-diyl.

The term “alkynyl” refers to an unsaturated, acyclic hydrocarbon radical, linear or branched, in so much as it contains one or more triple bonds. The alkynyl group can be optionally substituted with any moiety that does not adversely affect the reaction process, including but not limited to those described above for alkyl moeities. Non-limiting examples of suitable alkynyl groups include ethynyl, propynyl, hydroxypropynyl, butyn-1-yl, butyn-2-yl, pentyn-1-yl, pentyn-2-yl, 4-methoxypentyn-2-yl, 3-methylbutyn-1-yl, hexyn-1-yl, hexyn-2-yl, and hexyn-3-yl, 3,3-dimethylbutyn-1-yl radicals.

The term “alkylamino” or “arylamino” refers to an amino group that has one or two alkyl or aryl substituents, respectively.

The term “protected” as used herein and unless otherwise defined refers to a group that is added to an oxygen, nitrogen, or phosphorus atom to prevent its further reaction or for other purposes. A wide variety of oxygen and nitrogen protecting groups are known to those skilled in the art of organic synthesis, and are described, for example, in Greene et al., Protective Groups in Organic Synthesis, supra.

The term “aryl”, alone or in combination, means a carbocyclic aromatic system containing one, two or three rings wherein such rings can be attached together in a pendent manner or can be fused. Non-limiting examples of aryl include phenyl, biphenyl, or naphthyl, or other aromatic groups that remain after the removal of a hydrogen from an aromatic ring. The term aryl includes both substituted and unsubstituted moieties. The aryl group can be optionally substituted with any moiety that does not adversely affect the process, including but not limited to but not limited to those described above for alkyl moieties. Non-limiting examples of substituted aryl include heteroarylamino, N-aryl-N-alkylamino, N-heteroarylamino-N-alkylamino, heteroaralkoxy, arylamino, aralkylamino, arylthio, monoarylamidosulfonyl, arylsulfonamido, diarylamidosulfonyl, monoaryl amidosulfonyl, arylsulfinyl, arylsulfonyl, heteroarylthio, heteroarylsulfinyl, heteroarylsulfonyl, aroyl, heteroaroyl, aralkanoyl, heteroaralkanoyl, hydroxyaralkyl, hydroxyheteroaralkyl, haloalkoxyalkyl, aryl, aralkyl, aryloxy, aralkoxy, aryloxyalkyl, saturated heterocyclyl, partially saturated heterocyclyl, heteroaryl, heteroaryloxy, heteroaryloxyalkyl, arylalkyl, heteroarylalkyl, arylalkenyl, and heteroarylalkenyl, carboaralkoxy.

The terms “alkaryl” or “alkylaryl” refer to an alkyl group with an aryl substituent. The terms “aralkyl” or “arylalkyl” refer to an aryl group with an alkyl substituent.

The term “halo,” as used herein, includes chloro, bromo, iodo and fluoro.

The term “acyl” refers to a carboxylic acid ester in which the non-carbonyl moiety of the ester group is selected from straight, branched, or cyclic alkyl or lower alkyl, alkoxyalkyl including but not limited to methoxymethyl, aralkyl including but not limited to benzyl, aryloxyalkyl such as phenoxymethyl, aryl including but not limited to phenyl optionally substituted with halogen (F, Cl, Br, I), alkyl (including but not limited to C₁, C₂, C₃, and C₄) or alkoxy (including but not limited to C₁, C₂, C₃, and C₄), sulfonate esters such as alkyl or aralkyl sulphonyl including but not limited to methanesulfonyl, the mono, di or triphosphate ester, trityl or monomethoxytrityl, substituted benzyl, trialkylsilyl (e.g., dimethyl-t-butylsilyl) or diphenylmethylsilyl. Aryl groups in the esters optimally comprise a phenyl group. The term “lower acyl” refers to an acyl group in which the non-carbonyl moiety is lower alkyl.

The terms “alkoxy” and “alkoxyalkyl” embrace linear or branched oxy-containing radicals having alkyl moieties, such as methoxy radical. The term “alkoxyalkyl” also embraces alkyl radicals having one or more alkoxy radicals attached to the alkyl radical, that is, to form monoalkoxyalkyl and dialkoxyalkyl radicals. The “alkoxy” radicals can be further substituted with one or more halo atoms, such as fluoro, chloro or bromo, to provide “haloalkoxy” radicals. Examples of such radicals include fluoromethoxy, chloromethoxy, trifluoromethoxy, difluoromethoxy, trifluoroethoxy, fluoroethoxy, tetrafluoroethoxy, pentafluoroethoxy, and fluoropropoxy.

The term “alkylamino” denotes “monoalkylamino” and “dialkylamino” containing one or two alkyl radicals, respectively, attached to an amino radical. The terms arylamino denotes “monoarylamino” and “diarylamino” containing one or two aryl radicals, respectively, attached to an amino radical. The term “aralkylamino”, embraces aralkyl radicals attached to an amino radical. The term aralkylamino denotes “monoaralkylamino” and “diaralkylamino” containing one or two aralkyl radicals, respectively, attached to an amino radical. The term aralkylamino further denotes “monoaralkyl monoalkylamino” containing one aralkyl radical and one alkyl radical attached to an amino radical.

The term “heteroatom,” as used herein, refers to oxygen, sulfur, nitrogen and phosphorus.

The terms “heteroaryl” or “heteroaromatic,” as used herein, refer to an aromatic that includes at least one sulfur, oxygen, nitrogen or phosphorus in the aromatic ring.

The term “heterocyclic,” “heterocyclyl,” and “cycloheteroalkyl” refer to a nonaromatic cyclic group, for example, including between 3 and 10 atoms in the ring, wherein there is at least one heteroatom, such as oxygen, sulfur, nitrogen, or phosphorus in the ring.

Nonlimiting examples of heteroaryl and heterocyclic groups include furyl, furanyl, pyridyl, pyrimidyl, thienyl, isothiazolyl, imidazolyl, tetrazolyl, pyrazinyl, benzofuranyl, benzothiophenyl, quinolyl, isoquinolyl, benzothienyl, isobenzofuryl, pyrazolyl, indolyl, isoindolyl, benzimidazolyl, purinyl, carbazolyl, oxazolyl, thiazolyl, isothiazolyl, 1,2,4-thiadiazolyl, isooxazolyl, pyrrolyl, quinazolinyl, cinnolinyl, phthalazinyl, xanthinyl, hypoxanthinyl, thiophene, furan, pyrrole, isopyrrole, pyrazole, imidazole, 1,2,3-triazole, 1,2,4-triazole, oxazole, isoxazole, thiazole, isothiazole, pyrimidine or pyridazine, and pteridinyl, aziridines, thiazole, isothiazole, 1,2,3-oxadiazole, thiazine, pyridine, pyrazine, piperazine, pyrrolidine, oxaziranes, phenazine, phenothiazine, morpholinyl, pyrazolyl, pyridazinyl, pyrazinyl, quinoxalinyl, xanthinyl, hypoxanthinyl, pteridinyl, 5-azacytidinyl, 5-azauracilyl, triazolopyridinyl, imidazolopyridinyl, pyrrolopyrimidinyl, pyrazolopyrimidinyl, adenine, N⁶-alkylpurines, N⁶-benzylpurine, N⁶-halopurine, N⁶-vinypurine, N⁶-acetylenic purine, N⁶-acyl purine, N⁶-hydroxyalkyl purine, N⁶-thioalkyl purine, thymine, cytosine, 6-azapyrimidine, 2-mercaptopyrmidine, uracil, N⁵-alkylpyrimidines, N⁵-benzylpyrimidines, N⁵-halopyrimidines, N⁵-vinylpyrimidine, N⁵-acetylenic pyrimidine, N⁵-acyl pyrimidine, N⁵-hydroxyalkyl purine, and N⁶-thioalkyl purine, and isoxazolyl. The heteroaromatic group can be optionally substituted as described above for aryl. The heterocyclic or heteroaromatic group can be optionally substituted with one or more substituent selected from halogen, haloalkyl, alkyl, alkoxy, hydroxy, carboxyl derivatives, amido, amino, alkylamino, dialkylamino. The heteroaromatic can be partially or totally hydrogenated as desired. As a nonlimiting example, dihydropyridine can be used in place of pyridine. Functional oxygen and nitrogen groups on the heterocyclic or heteroaryl group can be protected as necessary or desired. Suitable protecting groups are well known to those skilled in the art, and include trimethylsilyl, dimethylhexylsilyl, t-butyldimethylsilyl, and t-butyldiphenylsilyl, trityl or substituted trityl, alkyl groups, acyl groups such as acetyl and propionyl, methanesulfonyl, and p-toluenelsulfonyl. The heterocyclic or heteroaromatic group can be substituted with any moiety that does not adversely affect the reaction, including but not limited to but not limited to those described above for aryl.

The term “host,” as used herein, refers to a unicellular or multicellular organism in which the virus can replicate, including but not limited to cell lines and animals, and, preferably, humans. Alternatively, the host can be carrying a part of the viral genome, whose replication or function can be altered by the compounds of the present invention. The term host specifically refers to infected cells, cells transfected with all or part of the viral genome and animals, in particular, primates (including but not limited to chimpanzees) and humans. In most animal applications of the present invention, the host is a human patient. Veterinary applications, in certain indications, however, are clearly contemplated by the present invention (such as for use in treating chimpanzees).

The term “peptide” refers to a various natural or synthetic compound containing two to one hundred amino acids linked by the carboxyl group of one amino acid to the amino group of another.

The term “pharmaceutically acceptable salt or prodrug” is used throughout the specification to describe any pharmaceutically acceptable form (such as an ester, phosphate ester, salt of an ester or a related group) of a nucleotide compound which, upon administration to a patient, provides the nucleotide monophosphate compound. Pharmaceutically acceptable salts include those derived from pharmaceutically acceptable inorganic or organic bases and acids. Suitable salts include those derived from alkali metals such as potassium and sodium, alkaline earth metals such as calcium and magnesium, among numerous other acids well known in the pharmaceutical art. Pharmaceutically acceptable prodrugs refer to a compound that is metabolized, for example hydrolyzed or oxidized, in the host to form the compound of the present invention. Typical examples of prodrugs include compounds that have biologically labile protecting groups on functional moieties of the active compound. Prodrugs include compounds that can be oxidized, reduced, aminated, deaminated, hydroxylated, dehydroxylated, hydrolyzed, dehydrolyzed, alkylated, dealkylated, acylated, deacylated, phosphorylated, or dephosphorylated to produce the active compound. The prodrug forms of the compounds of this invention can possess antiviral activity, can be metabolized to form a compound that exhibits such activity, or both.

As used herein, the term “resistant virus” refers to a virus that exhibits a three, and more typically, five or greater fold increase in EC₅₀ compared to naive virus in a constant cell line, including, but not limited to peripheral blood mononuclear (PBM) cells, or MT2 or MT4 cells.

As used herein, the term DAPD ((2R,4R)-2-amino-9-[(2-hydroxymethyl)-I, 3-dioxolan-4-yl]adenine) is also intended to include a related form of DAPD known as APD [(−)-β-D-2-aminopurine dioxolane].

The term “antiviral thymidine nucleosides” refers to thymidine analogues with anti-HIV activity, including but not limited to, AZT (zidovudine) and D4T (2′,3′-didehydro-3′ deoxythymidine (stravudine), and 1-β-D-Dioxolane)thymine (DOT) or their prodrugs.

The term AZT is used interchangeably with the term zidovudine throughout. Similarly, abbreviated and common names for other antiviral agents are used interchangeably throughout.

### II. ACTIVE COMPOUND

In one embodiment of the invention, the active compound is

The compounds described herein include monophosphate, phosphonate, and other analogs of β-D-6-substituted-2-amino purine dioxolanes.

In one embodiment, the active compound is of one of the following formulas:

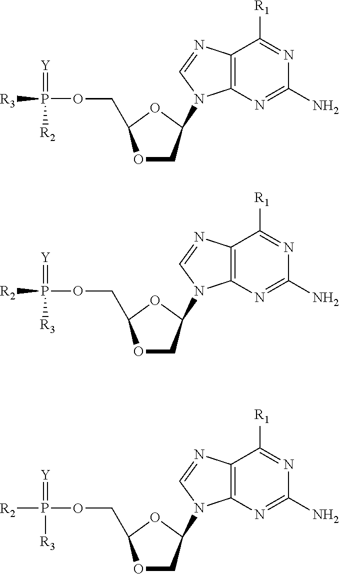

or a pharmaceutically acceptable salt or prodrug thereof, wherein:
ii) R′ is an atom or group removed in vivo to form OH when administered as the parent nucleoside, for example, halogen (F, Cl, Br, I), OR′, N(R′)₂, SR′, OCOR′, NHCOR′, N(COR′)COR′, SCOR′, OCOOR′, and NHCOOR′.
   each R′ is independently H, a lower alkyl (C₁-C₆), lower haloalkyl (C₁-C₆), lower alkoxy (C₁-C₆), lower alkenyl (C₂-C₆), lower alkynyl (C₂-C₆), lower cycloalkyl (C₃-C₆) aryl, heteroaryl, alkylaryl, or arylalkyl, wherein the groups can be substituted with one or more substituents as defined above, for example, hydroxyalkyl, aminoalkyl, and alkoxyalkyl.
Y is O or S;
R² and R³, when administered in vivo, are ideally capable of providing the nucleoside monophosphate monophosphonate, thiomonophosphonate, or thiomonophosphate. Representative R² and R³ are independently selected from:
   (a) OR⁸ where R⁸ is H, C₁₋₂₀ alkyl, C₃₋₆ cycloalkyl, C₁₋₆ haloalkyl, aryl, or heteroaryl which includes, but is not limited to, phenyl or naphthyl optionally substituted with one to three substituents independently selected from the group consisting of C₁₋₆ alkyl, C₂₋₆ alkenyl, C₂₋₆ alkynyl, C₁₋₆ alkoxy, (CH₂)₁₋₆CO₂R^{9a}, halogen, C₁₋₆ haloalkyl, —N(R^{9a})₂, C₁₋₆ acylamino, —NHSO₂C₁₋₆ alkyl, —SO₂N(R^{9a})₂, —SO₂C₁₋₆ alkyl, COR^{9b}, nitro and cyano;
   R^{9a} is independently H or C₁₋₆ alkyl;
   R^{9b} is —OR^{9a} or —N(R^{9a})₂;
(b)

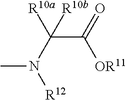

where R^{10a} and R^{10b} are:
(i) independently selected from the group consisting of H, C₁₋₁₀ alkyl, —(CH₂)ᵣNR^{9a}₂, C₁₋₆ hydroxyalkyl, —CH₂SH, —(CH₂)₂S(O)ₚMe, —(CH₂)₃NHC(═NH)NH₂, (1H-indol-3-yl)methyl, (1H-imidazol-4-yl)methyl, —(CH₂)ₘCOR^{9b}, aryl and aryl-C₁₋₃ alkyl, said aryl groups optionally substituted with a group selected from the group consisting of hydroxyl, C₁₋₁₀ alkyl, C₁₋₆ alkoxy, halogen, nitro, and cyano;
(ii) R^{10a} is H and R^{10b} and R¹² together are (CH₂)₂₋₄ to form a ring that includes the adjoining N and C atoms;
(iii) R^{10a} and R^{10b} together are (CH₂)₂ to form a ring;
(iv) R^{10a} and R^{10b} both are C₁₋₆ alkyl; or
(v) R^{10a} is H and R^{10b} is H, CH₃, CH₂CH₃, CH(CH₃)₂, CH₂CH(CH₃)₂, CH(CH₃)CH₂CH₃, CH₂Ph, CH₂-indol-3-yl, —CH₂CH₂SCH₃, CH₂CO₂H, CH₂C(O)NH₂, CH₂CH₂COOH, CH₂CH₂C(O)NH₂, CH₂CH₂CH₂CH₂NH₂—CH₂CH₂CH₂NHC(NH)NH₂, CH₂-imidazol-4-yl, CH₂OH, CH(OH)CH₃, CH₂((4′-OH)-Ph), CH₂SH, or lower cycloalkyl;
p is 0 to 2;
r is 1 to 6;
n is 4 or 5;
m is 0 to 3;
   R¹¹ is H, C₁₋₁₀ alkyl, or C₁₋₁₀ alkyl substituted with a lower alkyl, alkoxy, di(lower alkyl)-amino, fluoro, C₃₋₁₀ cycloalkyl, cycloalkyl alkyl, cycloheteroalkyl, aryl, such as phenyl, heteroaryl, such as, pyridinyl, substituted aryl, or substituted heteroaryl; wherein the substituents are C₁₋₅ alkyl, or C₁₋₅ alkyl substituted with a lower alkyl, alkoxy, di(lower alkyl)-amino, fluoro, C₃₋₁₀ cycloalkyl, or cycloalkyl;
   R¹² is H, C₁₋₃ alkyl, or R^{10a}, or R^{10b} and R¹² together are (CH₂)₂₋₄ so as to form a ring that includes the adjoining N and C atoms;
(c) an O attached lipid (including a phospholipid), an N or O attached peptide, an O attached cholesterol, or an O attached phytosterol;
(d) R² and R³ may come together to form a ring

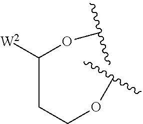

where W² is selected from a group consisting of phenyl or monocyclic heteroaryl, optionally substituted with one to three substituents independently selected from the group consisting of C₁₋₆ alkyl, CF₃, C₂₋₆ alkenyl, C₁₋₆ alkoxy, OR^{9c}, CO₂R^{9a}, COR^{9a}, halogen, C₁₋₆ haloalkyl, —N(R^{9a})₂, C₁₋₆ acylamino, CO₂N(R^{9a})₂, SR^{9a}, —NHSO₂C₁₋₆ alkyl, —SO₂N(R^{9a})₂, —SO₂C₁₋₆ alkyl, COR^{9b}, and cyano, and wherein said monocyclic heteroaryl and substituted monocyclic heteroaryl has 1-2 heteroatoms that are independently selected from the group consisting of N, O, and S with the provisos that:
a) when there are two heteroatoms and one is O, then the other can not be O or S, and
b) when there are two heteroatoms and one is S, then the other can not be O or S;
R^{9a} is independently H or C₁₋₆ alkyl;
R^{9b} is —OR^{9a} or —N(R^{9a})₂;
R^{9c} is H or C₁₋₆ acyl;
(e)

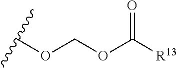

where R¹³ is selected from a group consisting of H, C₁₋₁₀ alkyl, C₁₋₁₀ alkyl optionally substituted with a lower alkyl, alkoxy, di(lower alkyl)-amino, fluoro, C₃₋₁₀ cycloalkyl, cycloalkyl alkyl, cycloheteroalkyl, aryl, such as phenyl, heteroaryl, such as, pyridinyl, substituted aryl, or substituted heteroaryl; wherein the substituents are C₁₋₅ alkyl, or C₁₋₅ alkyl substituted with a lower alkyl, alkoxy, di(lower alkyl)-amino, fluoro, C₃₋₁₀ cycloalkyl, or cycloalkyl;
f) R² and R³ may come together to form a ring

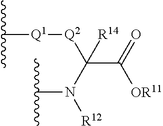

where R¹⁴ is: (i) independently selected from the group consisting of H, C₁₋₁₀ alkyl, —(CH₂)ᵣNR₂^{9a}, C₁₋₆ hydroxyalkyl, —CH₂SH, —(CH₂)₂S(O)ₚMe, —(CH₂)₃NHC(═NH)NH₂, (1H-indol-3-yl)methyl, (1H-imidazol-4-yl)methyl, —(CH₂)ₘCOR^{9b}, aryl and aryl-C₁₋₃ alkyl or heteroaryl and heteroaryl-C₁₋₃ alkyl, said aryl and heteroaryl groups optionally substituted with a group selected from the group consisting of hydroxyl, C₁₋₁₀ alkyl, C₁₋₆ alkoxy, halogen, nitro, and cyano; (ii) R¹⁴ is H, CH₃, CH₂CH₃, CH(CH₃)₂, CH₂CH(CH₃)₂, CH(CH₃)CH₂CH₃, CH₂Ph, CH₂-indol-3-yl, —CH₂CH₂SCH₃, CH₂CO₂H, CH₂C(O)NH₂, CH₂CH₂COOH, CH₂CH₂C(O)NH₂, CH₂CH₂CH₂CH₂NH₂, CH₂CH₂CH₂NHC(NH)NH₂, CH₂-imidazol-4-yl, CH₂OH, CH(OH)CH₃, CH₂((4′-OH)-Ph), CH₂SH, or lower cycloalkyl;
p is 0 to 2;
r is 1 to 6;
m is 0 to 3
Q¹ is NR^{9a}, O, or S
Q² is C₁₋₁₀ alkyl, C₁₋₆ hydroxyalkyl, aryl and aryl-C₁₋₃ alkyl, heteroaryl and heteroaryl-C₁₋₃ alkyl, said aryl and heteroaryl groups optionally substituted with a group selected from the group consisting of hydroxyl, C₁₋₁₀ alkyl, C₁₋₆ alkoxy, fluoro, and chloro;
   R¹¹ is H, C₁₋₁₀ alkyl, C₁₋₁₀ alkyl optionally substituted with a lower alkyl, alkoxy, di(lower alkyl)-amino, fluoro, C₃₋₁₀ cycloalkyl, cycloalkyl alkyl, cycloheteroalkyl, aryl, such as phenyl, heteroaryl, such as, pyridinyl, substituted aryl, or substituted heteroaryl; wherein the substituents are C₁₋₅ alkyl, or C₁₋₅ alkyl substituted with a lower alkyl, alkoxy, di(lower alkyl)-amino, fluoro, C₃₋₁₀ cycloalkyl, or cycloalkyl;
   R¹² is H, or C₁₋₃ alkyl, or R^{14b} and R¹² together are (CH₂)₂₋₄ so as to form a ring that includes the adjoining N and C atoms;

In one embodiment, the compounds have one of the following formulas:

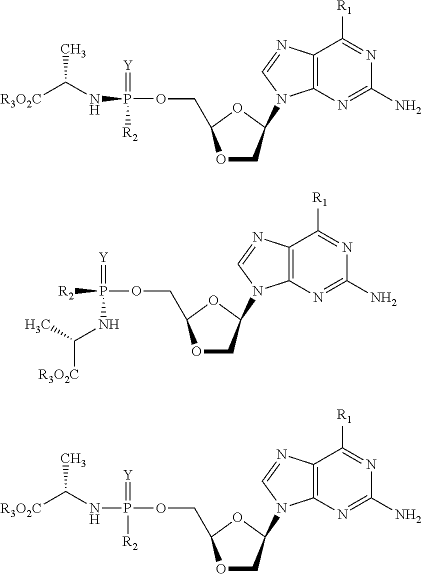

The first two compounds show both enantiomers of the phosphorus atom. Coupled with the chiral carbon on the dioxolane, these compounds exist as diastereomers. The third compound shows a racemic phosphorus, and a chiral dioxolane. In all three compounds, the amino acid attached to the phosphorus is L-alanine, or an ester derivative thereof.

In one embodiment, R₁ is selected from the group consisting of halo (i.e., Cl, Br, I, and F), NH₂, OMe, and NH—C₃₋₆ cycloalkyl. In another embodiment, R₁ is selected from the group consisting of Cl, NH₂, OMe, and NH—C₃ cycloalkyl.

The prodrug compounds described herein are in the form of the β-D-configuration, or at least the β-D-configuration is the major configuration, with an enantiomeric excess greater than 95%, preferably greater than 97%. Where one or R₂ and R₃ is alanine or an alanine ester, with the nitrogen attached to the phosphorus, the alanine is preferably in the L configuration.

The compounds described herein are preferably in the form of the β-D-configuration, although in one embodiment, can also be in the form of the β-L-configuration, or a mixture thereof, including a racemic mixture thereof.

### III. STEREOISOMERISM AND POLYMORPHISM

The compounds described herein may have asymmetric centers and occur as racemates, racemic mixtures, individual diastereomers or enantiomers, with all isomeric forms being included in the present invention. Compounds of the present invention having a chiral center can exist in and be isolated in optically active and racemic forms. Some compounds can exhibit polymorphism. The present invention encompasses racemic, optically-active, polymorphic, or stereoisomeric forms, or mixtures thereof, of a compound of the invention, which possess the useful properties described herein. The optically active forms can be prepared by, for example, resolution of the racemic form by recrystallization techniques, by synthesis from optically-active starting materials, by chiral synthesis, or by chromatographic separation using a chiral stationary phase or by enzymatic resolution. One can either purify the respective nucleoside, then derivatize the nucleoside to form the compounds described herein, or purify the nucleotides themselves.

Optically active forms of the compounds can be prepared using any method known in the art, including but not limited to by resolution of the racemic form by recrystallization techniques, by synthesis from optically-active starting materials, by chiral synthesis, or by chromatographic separation using a chiral stationary phase.

Examples of methods to obtain optically active materials include at least the following.
i) physical separation of crystals: a technique whereby macroscopic crystals of the individual enantiomers are manually separated. This technique can be used if crystals of the separate enantiomers exist, i.e., the material is a conglomerate, and the crystals are visually distinct;
ii) simultaneous crystallization: a technique whereby the individual enantiomers are separately crystallized from a solution of the racemate, possible only if the latter is a conglomerate in the solid state;
iii) enzymatic resolutions: a technique whereby partial or complete separation of a racemate by virtue of differing rates of reaction for the enantiomers with an enzyme;
iv) enzymatic asymmetric synthesis: a synthetic technique whereby at least one step of the synthesis uses an enzymatic reaction to obtain an enantiomerically pure or enriched synthetic precursor of the desired enantiomer;
v) chemical asymmetric synthesis: a synthetic technique whereby the desired enantiomer is synthesized from an achiral precursor under conditions that produce asymmetry (i.e., chirality) in the product, which can be achieved using chiral catalysts or chiral auxiliaries;
vi) diastereomer separations: a technique whereby a racemic compound is reacted with an enantiomerically pure reagent (the chiral auxiliary) that converts the individual enantiomers to diastereomers. The resulting diastereomers are then separated by chromatography or crystallization by virtue of their now more distinct structural differences and the chiral auxiliary later removed to obtain the desired enantiomer;
vii) first- and second-order asymmetric transformations: a technique whereby diastereomers from the racemate equilibrate to yield a preponderance in solution of the diastereomer from the desired enantiomer or where preferential crystallization of the diastereomer from the desired enantiomer perturbs the equilibrium such that eventually in principle all the material is converted to the crystalline diastereomer from the desired enantiomer. The desired enantiomer is then released from the diastereomer;
viii) kinetic resolutions: this technique refers to the achievement of partial or complete resolution of a racemate (or of a further resolution of a partially resolved compound) by virtue of unequal reaction rates of the enantiomers with a chiral, non-racemic reagent or catalyst under kinetic conditions;
ix) enantiospecific synthesis from non-racemic precursors: a synthetic technique whereby the desired enantiomer is obtained from non-chiral starting materials and where the stereochemical integrity is not or is only minimally compromised over the course of the synthesis;
x) chiral liquid chromatography: a technique whereby the enantiomers of a racemate are separated in a liquid mobile phase by virtue of their differing interactions with a stationary phase (including but not limited to via chiral HPLC). The stationary phase can be made of chiral material or the mobile phase can contain an additional chiral material to provoke the differing interactions;
xi) chiral gas chromatography: a technique whereby the racemate is volatilized and enantiomers are separated by virtue of their differing interactions in the gaseous mobile phase with a column containing a fixed non-racemic chiral adsorbent phase;
xii) extraction with chiral solvents: a technique whereby the enantiomers are separated by virtue of preferential dissolution of one enantiomer into a particular chiral solvent;
xiii) transport across chiral membranes: a technique whereby a racemate is placed in contact with a thin membrane barrier. The barrier typically separates two miscible fluids, one containing the racemate, and a driving force such as concentration or pressure differential causes preferential transport across the membrane barrier. Separation occurs as a result of the non-racemic chiral nature of the membrane that allows only one enantiomer of the racemate to pass through.

Chiral chromatography, including but not limited to simulated moving bed chromatography, is used in one embodiment. A wide variety of chiral stationary phases are commercially available. Chiral chromatography can also be used to isolate enantiomerically-enriched compounds where the phosphorus atom is chiral (i.e., R₂≠R₃).

In addition to the techniques described herein for producing enantiomerically-enriched compounds, where the chirality exists on a carbon, the phosphorous-containing prodrugs herein have a potentially chiral phosphorus atom (i.e., when R₂≠R₃), which can also be enantiomerically enriched.

Suitable techniques for providing enantiomerically-enriched chiral phosphorous atoms in the prodrug compounds described herein are known to those of skill in the art, and are described, for example, in Koziolkiewicz et al., Nucleic Acids Research, 1995, Vol. 23, No. 24 5001. As with enzymatic approaches to enantiomerically-enrich chiral carbons, there are also enzymes, such as Rp-specific snake venom phosphodiesterase (svPDE). For example, a racemic mixture can be incubated with svPDE at 37° C. for 24 hours (see, for example, Eckstein et al., J. Biol. Chem. 254:7476-7478 (1979) and Benkovic and Bryand, Biochemistry, 18:2825-2828 (1979). Alternatively, there is an Sp-specific nuclease P1 (see, for example, Potter et al., Biochemistry, 22:1369-1377 (1983), which can be used to prepare the other enantiomer.

### IV. NUCLEOTIDE SALT OR PRODRUG FORMULATIONS

In cases where compounds are sufficiently basic or acidic to form stable nontoxic acid or base salts, administration of the compound as a pharmaceutically acceptable salt may be appropriate. Examples of pharmaceutically acceptable salts are organic acid addition salts formed with acids, which form a physiological acceptable anion, for example, tosylate, methanesulfonate, acetate, citrate, malonate, tartarate, succinate, benzoate, ascorbate, α-ketoglutarate and α-glycerophosphate. Suitable inorganic salts can also be formed, including but not limited to, sulfate, nitrate, bicarbonate and carbonate salts.

Pharmaceutically acceptable salts can be obtained using standard procedures well known in the art, for example by reacting a sufficiently basic compound such as an amine with a suitable acid, affording a physiologically acceptable anion. Alkali metal (e.g., sodium, potassium or lithium) or alkaline earth metal (e.g., calcium) salts of carboxylic acids can also be made.

The nucleotide prodrugs described herein can be administered to additionally increase the activity, bioavailability, stability or otherwise alter the properties of the nucleotide monophosphate.

A number of nucleotide prodrug ligands are known. In general, alkylation, acylation or other lipophilic modification of the monophosphate or other analog of the nucleoside will increase the stability of the nucleotide.

Examples of substituent groups that can replace one or more hydrogens on the monophosphate moiety are alkyl, aryl, steroids, carbohydrates, including but not limited to sugars, 1,2-diacylglycerol and alcohols. Many are described in R. Jones & N. Bischofberger, Antiviral Research, 1995, 27, 1-17 and S. J. Hecker & M. D. Erion, J. Med. Chem., 2008, 51, 2328-2345. Any of these can be used in combination with the disclosed nucleotides to achieve a desired effect.

The active nucleotide can also be provided as a 5′-phosphoether lipid as disclosed in the following references, which are incorporated by reference: Kucera, L. S., N. Iyer, E. Leake, A. Raben, Modest E. K., D. L. W., and C. Piantadosi, “Novel membrane-interactive ether lipid analogs that inhibit infectious HIV-1 production and induce defective virus formation,” AIDS Res. Hum. Retroviruses, 1990, 6, 491-501; Piantadosi, C., J. Marasco C. J., S. L. Morris-Natschke, K. L. Meyer, F. Gumus, J. R. Surles, K. S. Ishaq, L. S. Kucera, N. Iyer, C. A. Wallen, S. Piantadosi, and E. J. Modest, “Synthesis and evaluation of novel ether lipid nucleoside conjugates for anti-HIV activity,” J. Med. Chem., 1991, 34, 1408-14; Hosteller, K. Y., D. D. Richman, D. A. Carson, L. M. Stuhmiller, G. M. T. van Wijk, and H. van den Bosch, “Greatly enhanced inhibition of human immunodeficiency virus type 1 replication in CEM and HT4-6C cells by 3′-deoxythymidine diphosphate dimyristoylglycerol, a lipid prodrug of 3,-deoxythymidine,” Antimicrob. Agents Chemother., 1992, 36, 2025-29; Hostetler, K. Y., L. M. Stuhmiller, H. B. Lenting, H. van den Bosch, and D. D. Richman, “Synthesis and antiretroviral activity of phospholipid analogs of azidothymidine and other antiviral nucleosides.” J. Biol. Chem., 1990, 265, 61127.

Nonlimiting examples of US patents that disclose suitable lipophilic substituents that can be covalently incorporated into the nucleoside, preferably at R² and/or R³ position of the nucleotides described herein, or lipophilic preparations, include U.S. Pat. Nos. 5,149,794 (Yatvin et al.); 5,194,654 (Hostetler et al.), 5,223,263 (Hostetler et al.); 5,256,641 (Yatvin et al.); 5,411,947 (Hostetler et al.); 5,463,092 (Hostetler et al.); 5,543,389 (Yatvin et al.); 5,543,390 (Yatvin et al.); 5,543,391 (Yatvin et al.); and 5,554,728 (Basava et al.), all of which are incorporated by reference. Foreign patent applications that disclose lipophilic substituents that can be attached to nucleosides of the present invention, or lipophilic preparations, include WO 89/02733, WO 90/00555, WO 91/16920, WO 91/18914, WO 93/00910, WO 94/26273, WO 96/15132, EP 0 350 287, EP 93917054.4, and WO 91/19721.

### VI. METHODS OF TREATMENT

Hosts, including but not limited to humans, infected with HIV-1, HIV-2, HBV, or a gene fragment thereof, can be treated by administering to the patient an effective amount of the active compound or a pharmaceutically acceptable prodrug or salt thereof in the presence of a pharmaceutically acceptable carrier or diluent. The active materials can be administered by any appropriate route, for example, orally, parenterally, intravenously, intradermally, subcutaneously, or topically, in liquid or solid form.

The compounds can also be used to treat cancer. Patients that can be treated with the compounds described herein, and the pharmaceutically acceptable salts and prodrugs of these compounds, according to the methods of this invention include, for example, patients that have been diagnosed as having lung cancer, bone cancer, pancreatic cancer, skin cancer, cancer of the head and neck, cutaneous or intraocular melanoma, uterine cancer, ovarian cancer, rectal cancer or cancer of the anal region, stomach cancer, colon cancer, breast cancer, gynecologic tumors (e.g., uterine sarcomas, carcinoma of the fallopian tubes, carcinoma of the endometrium, carcinoma of the cervix, carcinoma of the vagina or carcinoma of the vulva), Hodgkin's disease, cancer of the esophagus, cancer of the small intestine, cancer of the endocrine system (e.g., cancer of the thyroid, parathyroid or adrenal glands), sarcomas of soft tissues, cancer of the urethra, cancer of the penis, prostate cancer, chronic or acute leukemia, solid tumors of childhood, lymphocytic lymphomas, cancer of the bladder, cancer of the kidney or ureter (e.g., renal cell carcinoma, carcinoma of the renal pelvis), or neoplasms of the central nervous system (e.g., primary CNS lymphoma, spinal axis tumors, brain stem gliomas or pituitary adenomas).

### VII. COMBINATION OR ALTERNATION THERAPY FOR TREATING HIV AND/OR HBV

In one embodiment, the compounds of the invention can be employed together with at least one other antiviral agent, chosen from entry inhibitors, reverse transcriptase inhibitors, protease inhibitors, and immune-based therapeutic agents.

For example, when used to treat or prevent HIV or HBV infection, the active compound or its prodrug or pharmaceutically acceptable salt can be administered in combination or alternation with another antiviral agent, such as anti-HIV or anti-HBV, agent, including, but not limited to, those of the formulae above. In general, in combination therapy, effective dosages of two or more agents are administered together, whereas during alternation therapy, an effective dosage of each agent is administered serially. The dosage will depend on absorption, inactivation and excretion rates of the drug, as well as other factors known to those of skill in the art. It is to be noted that dosage values will also vary with the severity of the condition to be alleviated. It is to be further understood that for any particular subject, specific dosage regimens and schedules should be adjusted over time according to the individual need and the professional judgment of the person administering or supervising the administration of the compositions. Nonlimiting examples of antiviral agents that can be used in combination with the compounds disclosed herein include those in the tables below.

### HIV Therapies

### Protease Inhibitors (PIs)

| | | | Experimental | Pharmaceutical |
|---|---|---|---|---|
| Brand Name | Generic Name | Abbreviation | Code | Company |
| Invirase ® | saquinavir | SQV (HGC) | Ro-31-8959 | Hoffmann-La Roche |
| | (Hard Gel Cap) | | | |
| Fortovase ® | saquinavir | SQV (SGC) | | Hoffmann-La Roche |
| | (Soft Gel Cap) | | | |
| Norvir ® | ritonavir | RTV | ABT-538 | Abbott Laboratories |
| Crixivan ® | indinavir | IDV | MK-639 | Merck & Co. |
| Viracept ® | nelfinavir | NFV | AG-1343 | Pfizer |
| Agenerase ® | amprenavir | APV | 141W94 or VX-478 | GlaxoSmithKline |
| Kaletra ® | lopinavir + | LPV | ABT-378/r | Abbott Laboratories |
| | ritonavir | | | |
| Lexiva ® | fosamprenavir | | GW-433908 or | GlaxoSmithKline |
| | | | VX-175 | |
| Aptivus ® | tripanavir | TPV | PNU-140690 | Boehringer Ingelheim |
| Reyataz ® | atazanavir | | BMS-232632 | Bristol-Myers Squibb |
| | brecanavir | | GW640385 | GlaxoSmithKline |
| Prezista ™ | darunavir | | TMC114 | Tibotec |

### HIV Therapies

### Nucleoside/Nucleotide Reverse

### Transcriptase Inhibitors (NRTIs)

| | Generic | | Experimental | Pharmaceutical |
|---|---|---|---|---|
| Brand Name | Name | Abbreviation | Code | Company |
| Retrovir ® | zidovudine | AZT or ZDV | | GlaxoSmithKline |
| Epivir ® | lamivudine | 3TC | | GlaxoSmithKline |
| Combivir ® | zidovudine + | AZT + 3TC | | GlaxoSmithKline |
| | lamivudine | | | |
| Trizivir ® | abacavir + | ABC + AZT + | | GlaxoSmithKline |
| | zidovudine + | 3TC | | |
| | lamivudine | | | |
| Ziagen ® | abacavir | ABC | 1592U89 | GlaxoSmithKline |
| Epzicom ™ | abacavir + | ABC + 3TC | | GlaxoSmithKline |
| | lamivudine | | | |
| Hivid ® | zalcitabine | ddC | | Hoffmann-La |
| | | | | Roche |
| Videx ® | didanosine: | ddI | BMY-40900 | Bristol-Myers |
| | buffered | | | Squibb |
| | versions | | | |
| Entecavir | baraclude | | | Bristol-Myers |
| | | | | Squibb |
| Videx ® EC | didanosine: | ddI | | Bristol-Myers |
| | delayed- | | | Squibb |
| | release | | | |
| | capsules | | | |
| Zerit ® | stavudine | d4T | BMY-27857 | Bristol-Myers |
| | | | | Squibb |
| Viread ™ | tenofovir | TDF or | | Gilead Sciences |
| | disoproxil | Bis(POC) | | |
| | fumarate (DF) | PMPA | | |
| Emtriva ® | emtricitabine | FTC | | Gilead Sciences |
| Truvada ® | Viread + | TDF + FTC | | Gilead Sciences |
| | Emtriva | | | |
| Atripla ™ | | TDF + FTC + | | Gilead/BMS/Merck |
| | | Sustiva ® | | |
| | amdoxovir | DAPD, | | RFS Pharma LLC |
| | | AMDX | | |
| apricitabine | AVX754 | | SPD 754 | Avexa Ltd |
| | Alovudine | FLT | MIV-310 | Boehringer |
| | Elvucitabine | L-FD4C | ACH-126443, | Achillion |
| | KP-1461 | | SN1461, | Koronis |
| | | | SN1212 | |
| | Racivir | RCV | | Pharmasset |
| Dexelvuecitabine | Reverset | D-D4FC | DPC 817 | Pharmasset |
| | | | GS9148 and | Gilead Sciences |
| | | | prodrugs | |
| | | | thereof | |

### HIV Therapies

### Non-Nucleoside Reverse

### Transcriptase Inhibitors (NNRTIs)

| Brand | | | Experimental | Pharmaceutical |
|---|---|---|---|---|
| Name | Generic Name | Abbreviation | Code | Company |
| Viramune ® | nevirapine | NVP | BI-RG-587 | Boehringer Ingelheim |
| Rescriptor ® | delavirdine | DLV | U-90152S/T | Pfizer |
| Sustiva ® | efavirenz | EFV | DMP-266 | Bristol-Myers Squibb |
| | (+)-calanolide | | | Sarawak Medichem |
| | A | | | |
| | capravirine | CPV | AG-1549 or S-1153 | Pfizer |
| | | | DPC-083 | Bristol-Myers Squibb |
| | | | TMC-125 | Tibotec-Virco Group |
| | | | TMC-278 | Tibotec-Virco Group |
| | | | IDX12899 | Idenix |
| | | | IDX12989 | idenix |

### HIV Therapies

### Other Classes of Drugs

| Brand | Generic | | Experimental | Pharmaceutical |
|---|---|---|---|---|
| Name | Name | Abbreviation | Code | Company |
| Viread ™ | tenofovir | TDF or | | Gilead Sciences |
| | disoproxil | Bis(POC) | | |
| | fumarate | PMPA | | |
| | (DF) | | | |

### Cellular Inhibitors

| Brand | Generic | | Experimental | Pharmaceutical |
|---|---|---|---|---|
| Name | Name | Abbreviation | Code | Company |
| Droxia ® | hydroxyurea | HU | | Bristol-Myers |
| | | | | Squibb |

### Entry Inhibitors (Including Fusion Inhibitors)

| Brand | Generic | | Experimental | Pharmaceutical |
|---|---|---|---|---|
| Name | Name | Abbreviation | Code | Company |
| Fuzeon ™ | enfuvirtide | | T-20 | Trimeris |
| | | | T-1249 | Trimeris |
| | | | AMD-3100 | AnorMED, Inc. |
| | CD4-IgG2 | | PRO-542 | Progenics |
| | | | | Pharmaceuticals |
| | | | BMS-488043 | Bristol-Myers |
| | | | | Squibb |
| | aplaviroc | | GSK-873, 140 | GlaxoSmithKline |
| | Peptide T | | | Advanced |
| | | | | Immuni T, Inc. |
| | | | TNX-355 | Tanox, Inc. |
| | maraviroc | | UK-427, 857 | Pfizer |

CXCR4 Inhibitor

| | | |
|---|---|---|
| AMD070 | AMD11070 | AnorMED, Inc. |

CCR5 antagonist

| | | | |
|---|---|---|---|
| vicriroc | SCH-D | SCH-417690 | Schering-Plough |

### HIV Therapies

### Immune-Based Therapies

| | | Abbre- | Experimental | Pharmaceutical |
|---|---|---|---|---|
| Brand Name | Generic Name | viation | Code | Company |
| Proleukin ® | aldesleukin, or | IL-2 | | Chiron |
| | Interleukin-2 | | | Corporation |
| Remune ® | HIV-1 | | AG1661 | The Immune |
| | Immunogen, or | | | Response |
| | Salk vaccine | | | Corporation |
| | | | HE2000 | HollisEden |
| | | | | Pharmaceuticals |

Combinations of the Prodrugs Described Herein with Thymidine Nucleoside Antiviral Agents

In another embodiment, the combinations include zidovudine (AZT) or other thymidine nucleoside antiretroviral agents, and the DAPD and other 6-substituted aminopurine dioxolanes described herein. In this embodiment, the dosage of AZT or other thymidine nucleoside antiretroviral agents can be the same as or lower than conventional dosages.

As discussed above with respect to the first embodiment, co-formulation of AZT with other antiviral nucleoside agents as a “resistance repellent” for the K65R mutation provides better therapy than either alone. AZT and other thymidine nucleoside antiviral agents are also associated with various mutations in the viral DNA, and, therefore resistance to AZT can develop. These mutations are known as thymidine analog mutations (TAMs).

Amdoxovir (AMDX; DAPD) has been well studied in six trials in close to 200 subjects. AZT is synergistic with DAPD and prevents selection of K65R and thymidine analog mutations (TAMs). That is, while the AZT reduces the ability of the virus to develop the K65R mutation following administration of DAPD, the DAPD reduces the ability of the virus to develop TAMs mutations following administration of AZT. Thus, the two agents administered together are superior to either administered alone, since they can each effectively reduce the presence of viral mutations that would render the other either ineffective or less effective as an anti-HIV agent. Since the prodrug compounds described herein provide the same active agent as DAPD (i.e., DXG), they are similarly efficacious in treating virus with TAMS mutations, and are similarly prone to development of the K65 mutation.

Further, the dosage of AZT can be reduced in a manner which reduces the amount of AZT monophosphate (AZT-MP) accumulation, while maintaining antiviral effect. Thus, while AZT can be administered in the conventional dosage of 300 mg bid, it can also be administered in a lower dosage (i.e., between around 100 and around 250 bid) can be effective, yet minimize the accumulation of toxic by-products such as the monophosphate form of the agents.

In a clinical study using DAPD at a dosage of 500 mg bid and a dosage of AZT of 300 mg bid or 200 bid for 10 days (results not shown), DAPD/AZT viral load decline indicated synergy, and the combination therapy was effective and well tolerated. It is believed that long term studies with lower dose AZT will demonstrate decreased toxicity as well, though this study was limited to 10 days.

In the study, the effect of the combination therapy on hemoglobin concentrations and mean corpuscular volume, an indicator of the susceptibility to bone marrow toxicity, was determined. Twenty-four subjects were enrolled in a study (shown in Example 3) using the dosages for DAPD and AZT discussed above. Hematological indices including hemoglobin (g/dl) and mean corpuscular volume (MCV, femtoliters) were measured over time, and the data showed that the trend in decrease in hemoglobin from Baseline was DAPD/AZT 300≧AZT 300≧DAPD/AZT 200>AZT 200>DAPD>placebo and the trend in increase in MCV from Baseline was DAPD/AZT 300>AZT 300>DAPD/AZT 200>AZT 20022 placebo>DAPD. The data shows that the lower dosage of AZT effectively lowered the incidence of side effects associated with bone marrow toxicity.

Replacement of DAPD with the prodrugs described herein will render the combination therapy even more effective, because the prodrugs are more effective than DAPD, and can thus be administered at a lower dosage.

In general, during alternation therapy, an effective dosage of each agent is administered serially, whereas in combination therapy, an effective dosage of two or more agents are administered together. In alternation therapy, for example, one or more first agents can be administered in an effective amount for an effective time period to treat the viral infection, and then one or more second agents substituted for those first agents in the therapy routine and likewise given in an effective amount for an effective time period.

The dosages will depend on such factors as absorption, biodistribution, metabolism and excretion rates for each drug as well as other factors known to those of skill in the art. It is to be noted that dosage values will also vary with the severity of the condition to be alleviated. It is to be further understood that for any particular subject, specific dosage regimens and schedules should be adjusted over time according to the individual need and the professional judgment of the person administering or supervising the administration of the compositions.

Examples of suitable dosage ranges for anti-HIV compounds, including thymidine nucleoside derivatives such as AZT and non-thymidine nucleoside derivatives such as 3TC, can be found in the scientific literature and in the Physicians Desk Reference. Many examples of suitable dosage ranges for other compounds described herein are also found in public literature or can be identified using known procedures. These dosage ranges can be modified as desired to achieve a desired result.

In one preferred embodiment, one or more of the prodrugs described herein are administered in combination or alternation with AZT.

Alkyl hydrogen phosphate derivatives of the anti-HIV agent AZT may be less toxic than the parent nucleoside analogue, and can be used in place of AZT. Antiviral Chem. Chemother. 5, 271 277; Meyer, R. B., Jr., Shuman, D. A. and Robins, R. K. (1973) “Synthesis of purine nucleoside 3′,5′-cyclic phosphoramidates.” Tetrahedron Lett. 269 272; Nagyvary, J. Gohil, R. N., Kirchner, C. R. and Stevens, J. D. (1973) “Studies on neutral esters of cyclic AMP,” Biochem. Biophys. Res. Commun. 55, 1072 1077; Namane, A. Gouyette, C., Fillion, M. P., Fillion, G. and Huynh-Dinh, T. (1992) “Improved brain delivery of AZT using a glycosyl phosphotriester prodrug.” J. Med. Chem. 35, 3039 3044; Nargeot, J. Nerbonne, J. M. Engels, J. and Leser, H. A. (1983) Natl. Acad. Sci. U.S.A. 80, 2395 2399; Nelson, K. A., Bentrude, W. G. Stser, W. N. and Hutchinson, J. P. (1987) “The question of chair-twist equilibria for the phosphate rings of nucleoside cyclic 3′,5′monophosphates. ¹HNMR and x-ray crystallographic study of the diastereomers of thymidine phenyl cyclic 3′,5′-monophosphate.” J. Am. Chem. Soc. 109, 4058 4064; Nerbonne, J. M., Richard, S., Nargeot, J. and Lester, H. A. (1984) “New photoactivatable cyclic nucleotides produce intracellular jumps in cyclic AMP and cyclic GMP concentrations.” Nature 301, 74 76; Neumann, J. M., Herv_, M., Debouzy, J. C., Guerra, F. I., Gouyette, C., Dupraz, B. and Huyny-Dinh, T. (1989) “Synthesis and transmembrane transport studies by NMR of a glucosyl phospholipid of thymidine.” J. Am. Chem. Soc. 111, 4270 4277; Ohno, R., Tatsumi, N., Hirano, M., Imai, K. Mizoguchi, H., Nakamura, T., Kosaka, M., Takatuski, K., Yamaya, T., Toyama K., Yoshida, T., Masaoka, T., Hashimoto, S., Ohshima, T., Kimura, I., Yamada, K. and Kimura, J. (1991) “Treatment of myelodysplastic syndromes with orally administered 1-β-D-arabinouranosylcytosine-5′ stearylphosphate.” Oncology 48, 451 455. Palomino, E., Kessle, D. and Horwitz, J. P. (1989) “A dihydropyridine carrier system for sustained delivery of 2′,3′ dideoxynucleosides to the brain.” J. Med. Chem. 32, 22 625; Perkins, R. M., Barney, S. Wittrock, R., Clark, P. H., Levin, R. Lambert, D. M., Petteway, S. R., Serafinowska, H. T., Bailey, S. M., Jackson, S., Hamden, M. R. Ashton, R., Sutton, D., Harvey, J. J. and Brown, A. G. (1993) “Activity of BRL47923 and its oral prodrug, SB203657A against a rauscher murine leukemia virus infection in mice.” Antiviral Res. 20 (Suppl. 1). 84; Piantadosi, C., Marasco, C. J., Jr., Norris-Natschke, S. L., Meyer, K. L., Gumus, F., Surles, J. R., Ishaq, K. S., Kucera, L. S. Iyer, N., Wallen, C. A., Piantadosi, S, and Modest, E. J. (1991) “Synthesis and evaluation of novel ether lipid nucleoside conjugates for anti-HIV-1 activity.” J. Med. Chem. 34, 1408 1414; Pompon, A., Lefebvre, I., Imbach, J. L., Kahn, S, and Farquhar, D. (1994). “Decomposition pathways of the mono- and bis(pivaloyloxymethyl) esters of azidothymidine-5′-monophosphate in cell extract and in tissue culture medium; an application of the ‘on-line ISRP-cleaning HPLC technique.” Antiviral Chem. Chemother. 5, 91 98; Postemark, T. (1974) “Cyclic AMP and cyclic GMP.” Annu. Rev. Pharmacol. 14, 23 33; Prisbe, E. J., Martin, J. C. M., McGhee, D. P. C., Barker, M. F., Smee, D. F. Duke, A. E., Matthews, T. R. and Verheyden, J. P. J. (1986) “Synthesis and antiherpes virus activity of phosphate an phosphonate derivatives of 9-[(1,3-dihydroxy-2-propoxy)methyl]guanine.” J. Med. Chem. 29, 671 675; Pucch, F., Gosselin, G., Lefebvre, I., Pompon, a., Aubertin, A. M. Dim, and Imbach, J. L. (1993) “Intracellular delivery of nucleoside monophosphate through a reductase-mediated activation process.” Antiviral Res. 22, 155 174; Pugaeva, V. P., Klochkeva, S. I., Mashbits, F. D. and Eizengart, R. S. (1969). “Toxicological assessment and health standard ratings for ethylene sulfide in the industrial atmosphere.” Gig. Trf. Prof. Zabol. 14, 47 48 (Chem. Abstr. 72, 212); Robins, R. K. (1984) “The potential of nucleotide analogs as inhibitors of Retro viruses and tumors.” Pharm. Res. 11 18; Rosowsky, A., Kim. S. H., Ross and J. Wick, M. M. (1982) “Lipophilic 5′-(alkylphosphate) esters of 1-β-D-arabinofuranosylcytosine and its N⁴-acyl and 2,2′-anhydro-3′-O-acyl derivatives as potential prodrugs.” J. Med. Chem. 25, 171 178; Ross, W. (1961) “Increased sensitivity of the walker turnout towards aromatic nitrogen mustards carrying basic side chains following glucose pretreatment.” Biochem. Pharm. 8, 235 240; Ryu, E. K., Ross, R. J. Matsushita, T., MacCoss, M., Hong, C. I. and West, C. R. (1982). “Phospholipid-nucleoside conjugates. 3. Synthesis and preliminary biological evaluation of 1-β-D-arabinofuranosylcytosine 5′diphosphate [−]. 2-diacylglycerols.” J. Med. Chem. 25, 1322 1329; Saffhill, R. and Hume, W. J. (1986) “The degradation of 5-iododeoxyuridine and 5-bromoethoxyuridine by serum from different sources and its consequences for the use of these compounds for incorporation into DNA.” Chem. Biol. Interact. 57, 347 355; Saneyoshi, M., Morozumi, M., Kodama, K., Machida, J., Kuninaka, A. and Yoshino, H. (1980) “Synthetic nucleosides and nucleotides. XVI. Synthesis and biological evaluations of a series of 1-β-D-arabinofuranosylcytosine 5′-alky or arylphosphates.” Chem. Pharm. Bull. 28, 2915 2923; Sastry, J. K., Nehete, P. N., Khan, S., Nowak, B. J., Plunkett, W., Arlinghaus, R. B. and Farquhar, D. (1992) “Membrane-permeable dideoxyuridine 5′-monophosphate analogue inhibits human immunodeficiency virus infection.” Mol. Pharmacol. 41, 441 445; Shaw, J. P., Jones, R. J. Arimilli, M. N., Louie, M. S., Lee, W. A. and Cundy, K. C. (1994) “Oral bioavailability of PMEA from PMEA prodrugs in male Sprague-Dawley rats.” 9th Annual AAPS Meeting. San Diego, Calif. (Abstract). Shuto, S., Ueda, S., Imamura, S., Fukukawa, K. Matsuda, A. and Ueda, T. (1987) “A facile one-step synthesis of 5′ phosphatidiylnucleosides by an enzymatic two-phase reaction.” Tetrahedron Lett. 28, 199 202; Shuto, S. Itoh, H., Ueda, S., Imamura, S., Kukukawa, K., Tsujino, M., Matsuda, A. and Ueda, T. (1988) Pharm. Bull. 36, 209 217.

When the treatment involves co-administration of AZT or other thymidine nucleoside antiviral agents and non-thymidine nucleoside antiviral agents that select for the K65R mutation, it is desirable that the patient has not already developed the K65R mutation. Although the AZT portion of the combination therapy will still be effective, the other agent will be less effective, and perhaps no longer effective.

When the treatment involves co-administration of AZT or other thymidine nucleoside antiviral agents and DAPD, it is desirable that the patient has not already developed the K65R mutation or TAMs. That is, if the patient already has TAMs, the AZT portion of the combination therapy will be less effective, and perhaps no longer effective, and if the patient already has already developed the K65R mutation, the DAPD will be less effective, and perhaps no longer effective.

Those of skill in the art can effectively follow the administration of these therapies, and the development of side effects and/or resistant viral strains, without undue experimentation.

### Hepatitis B Therapies

| | Drug | |
|---|---|---|
| Drug Name | Class | Company |
| Intron A | interferon | Schering-Plough |
| (interferon alfa-2b) | | |
| Pegasys | interferon | Roche |
| (Peginterferon | | |
| alfa-2a) | | |
| Epivir-HBV | nucleoside | GlaxoSmithKline |
| (lamivudine; 3TC) | analogue | |
| Hepsera (Adefovir | nucleotide | Gilead Sciences |
| Dipivoxil)″ | analogue | |
| Emtriva ® | nucleoside | Gilead |
| (emtricitabine; | analogue | Scienceshttp://www.hivandhepatitis.com/ |
| FTC) | | advertisement/triangle.html |
| Entecavir | nucleoside | Bristol-Myers Squibb |
| | analogue | |
| Clevudine | nucleoside | Pharmasset |
| (CLV, L-FMAU) | analogue | |
| ACH 126, 443 | nucleoside | Achillion Pharmaceuticals |
| (L-Fd4C) | analogue | |
| AM 365 | nucleoside | Amrad |
| | analogue | |
| Amdoxovir | nucleoside | RFS Pharma LLC |
| (AMDX, DAPD) | analogue | |
| LdT (telbivudine) | nucleoside | Idenix |
| | analogue | |
| CS-1220 | nucleoside | Emory University |
| | analogue | |
| Theradigm | Immune | Epimmune |
| | stimulant | |
| Zadaxin (thymosin) | Immune | SciClone |
| | stimulant | |
| EHT 899 | viral | Enzo Biochem |
| | protein | |
| Dexelvuecitabine/ | nucleoside | Pharmasset |
| Reverset/D-D4FC | analogue | |
| APD | nucleoside | RFS Pharma |
| | analogue | |
| HBV DNA vaccine | Immune | PowderJect (UK) |
| | stimulant | |
| MCC 478 | nucleoside | Eli Lilly |
| | analogue | |
| valLdC | nucleoside | Idenix |
| (valtorcitabine) | analogue | |
| ICN 2001 | nucleoside | ICN |
| | analogue | |
| Racivir | nucleoside | Pharmasset |
| | analogue | |
| Robustaflavone | nucleoside | Advanced Life Sciences |
| | analogue | |
| LM-019c | | Emory University |
| Penciclovir | nucleoside | |
| | analogue | |
| Famciclovir | | |
| DXG | nucleoside | |
| | analogue | |
| ara-AMP prodrugs | | |
| HBV/MF59 | | |
| HDP-P-acyclovir | nucleoside | |
| | analogue | |
| Hammerhead | | |
| ribozymes | | |
| Glycosidase | | |
| Inhibitors | | |
| Pegylated Interferon | | |
| Human Monoclonal | | |
| Antibodies | | |

### VIII. COMBINATION THERAPY FOR TREATING CANCER AND OTHER PROLIFERATIVE CONDITIONS

This invention also relates to a method of and to a pharmaceutical composition for inhibiting abnormal cellular proliferation, such as cancer, in a patient. The pharmaceutical compositions comprise an amount of a compound described herein, or a pharmaceutically acceptable salt or prodrug thereof, and an amount of one or more substances selected from anti-angiogenesis agents, signal transduction inhibitors, and antiproliferative agents.

Anti-angiogenesis agents, such as MMP-2 (matrix-metalloproteinase 2) inhibitors, MMP-9 (matrix-metalloproteinase 9) inhibitors, and COX-II (cyclooxygenase II) inhibitors, can be used in conjunction with a compound of formula I and pharmaceutical compositions described herein. Examples of useful COX-II inhibitors include CELEBREX™ (alecoxib), valdecoxib, and rofecoxib. Examples of useful matrix metalloproteinase inhibitors are described in WO 96/33172 (published Oct. 24, 1996), WO 96/27583 (published Mar. 7, 1996), European Patent Application No. 97304971.1 (filed Jul. 8, 1997), European Patent Application No. 99308617.2 (filed Oct. 29, 1999), WO 98/07697 (published Feb. 26, 1998), WO 98/03516 (published Jan. 29, 1998), WO 98/34918 (published Aug. 13, 1998), WO 98/34915 (published Aug. 13, 1998), WO 98/33768 (published Aug. 6, 1998), WO 98/30566 (published Jul. 16, 1998), European Patent Publication 606,046 (published Jul. 13, 1994), European Patent Publication 931,788 (published Jul. 28, 1999), WO 90/05719 (published May 331, 1990), WO 99/52910 (published Oct. 21, 1999), WO 99/52889 (published Oct. 21, 1999), WO 99/29667 (published Jun. 17, 1999), PCT International Application No. PCT/IB98/01113 (filed Jul. 21, 1998), European Patent Application No. 99302232.1 (filed Mar. 25, 1999), Great Britain patent application number 9912961.1 (filed Jun. 3, 1999), U.S. Provisional Application No. 60/148,464 (filed Aug. 12, 1999), U.S. Pat. No. 5,863,949 (issued Jan. 26, 1999), U.S. Pat. No. 5,861,510 (issued Jan. 19, 1999), and European Patent Publication 780,386 (published Jun. 25, 1997), all of which are incorporated herein in their entireties by reference. Preferred MMP inhibitors are those that do not demonstrate arthralgia. More preferred, are those that selectively inhibit MMP-2 and/or MMP-9 relative to the other matrix-metalloproteinases (i.e. MMP-1, MMP-3, MMP-4, MMP-5, MMP-6, MMP-7, MMP-8, MMP-10, MMP-11, MMP-12, and MMP-13).

The compounds described herein can also be used with signal transduction inhibitors, such as agents that can inhibit EGFR (epidermal growth factor receptor) responses, such as EGFR antibodies, EGF antibodies, and molecules that are EGFR inhibitors; VEGF (vascular endothelial growth factor) inhibitors, such as VEGF receptors and molecules that can inhibit VEGF; and erbB2 receptor inhibitors, such as organic molecules or antibodies that bind to the erbB2 receptor, for example, HERCEPTIN™ (Genentech, Inc. of South San Francisco, Calif., USA).

EGFR inhibitors are described in, for example in WO 95/19970 (published Jul. 27, 1995), WO 98/14451 (published Apr. 9, 1998), WO 98/02434 (published Jan. 22, 1998), and U.S. Pat. No. 5,747,498 (issued May 5, 1998), and such substances can be used in the present invention as described herein. EGFR-inhibiting agents include, but are not limited to, the monoclonal antibodies C225 and anti-EGFR 22Mab (ImClone Systems Incorporated of New York, N.Y., USA), ABX-EGF (Abgenix/Cell Genesys), EMD-7200 (Merck KgaA), EMD-5590 (Merck KgaA), MDX-447/H-477 (Medarex Inc. of Annandale, N.J., USA and Merck KgaA), and the compounds ZD-1834, ZD-1838 and ZD-1839 (AstraZeneca), PKI-166 (Novartis), PKI-166/CGP-75166 (Novartis), PTK 787 (Novartis), CP 701 (Cephalon), leflunomide (Pharmacia/Sugen), CI-1033 (Warner Lambert Parke Davis), CI-1033/PD 183,805 (Warner Lambert Parke Davis), CL-387,785 (Wyeth-Ayerst), BBR-1611 (Boehringer Mannheim GmbH/Roche), Naamidine A (Bristol Myers Squibb), RC-3940-II (Pharmacia), BIBX-1382 (Boehringer Ingelheim), OLX-103 (Merck & Co. of Whitehouse Station, N.J., USA), VRCTC-310 (Ventech Research), EGF fusion toxin (Seragen Inc. of Hopkinton, Mass.), DAB-389 (Seragen/Lilgand), ZM-252808 (Imperical Cancer Research Fund), RG-50864 (INSERM), LFM-A12 (Parker Hughes Cancer Center), WHI-P97 (Parker Hughes Cancer Center), GW-282974 (Glaxo), KT-8391 (Kyowa Hakko) and EGFR Vaccine (York Medical/Centro de Immunologia Molecular (CIM)). These and other EGFR-inhibiting agents can be used in the present invention.

VEGF inhibitors, for example CP-547,632 (Pfizer Inc., N.Y.), AG-13736 (Agouron Pharmaceuticals, Inc. a Pfizer Company), SU-5416 and SU-6668 (Sugen Inc. of South San Francisco, Calif., USA), and SH-268 (Schering) can also be combined with the compound of the present invention. VEGF inhibitors are described in, for example in WO 99/24440 (published May 20, 1999), PCT International Application PCT/IB99/00797 (filed May 3, 1999), in WO 95/21613 (published Aug. 17, 1995), WO 99/61422 (published Dec. 2, 1999), U.S. Pat. No. 5,834,504 (issued Nov. 10, 1998), WO 98/50356 (published Nov. 12, 1998), U.S. Pat. No. 5,883,113 (issued Mar. 16, 1999), U.S. Pat. No. 5,886,020 (issued Mar. 23, 1999), U.S. Pat. No. 5,792,783 (issued Aug. 11, 1998), WO 99/10349 (published Mar. 4, 1999), WO 97/32856 (published Sep. 12, 1997), WO 97/22596 (published Jun. 26, 1997), WO 98/54093 (published Dec. 3, 1998), WO 98/02438 (published Jan. 22, 1998), WO 99/16755 (published Apr. 8, 1999), and WO 98/02437 (published Jan. 22, 1998), all of which are incorporated herein in their entireties by reference. Other examples of some specific VEGF inhibitors useful in the present invention are IM862 (Cytran Inc. of Kirkland, Wash., USA); anti-VEGF monoclonal antibody of Genentech, Inc. of South San Francisco, Calif.; and angiozyme, a synthetic ribozyme from Ribozyme (Boulder, Colo.) and Chiron (Emeryville, Calif.). These and other VEGF inhibitors can be used in the present invention as described herein.

ErbB2 receptor inhibitors, such as CP-358,774 (OSI-774) (Tarceva) (OSI Pharmaceuticals, Inc.), GW-282974 (Glaxo Wellcome plc), and the monoclonal antibodies AR-209 (Aronex Pharmaceuticals Inc. of The Woodlands, Tex., USA) and 2B-1 (Chiron), can furthermore be combined with the compound of the invention, for example those indicated in WO 98/02434 (published Jan. 22, 1998), WO 99/35146 (published Jul. 15, 1999), WO 99/35132 (published Jul. 15, 1999), WO 98/02437 (published Jan. 22, 1998), WO 97/13760 (published Apr. 17, 1997), WO 95/19970 (published Jul. 27, 1995), U.S. Pat. No. 5,587,458 (issued Dec. 24, 1996), and U.S. Pat. No. 5,877,305 (issued Mar. 2, 1999), which are all hereby incorporated herein in their entireties by reference. ErbB2 receptor inhibitors useful in the present invention are also described in U.S. Provisional Application No. 60/117,341, filed Jan. 27, 1999, and in U.S. Provisional Application No. 60/117,346, filed Jan. 27, 1999, both of which are incorporated in their entireties herein by reference. The erbB2 receptor inhibitor compounds and substance described in the aforementioned PCT applications, U.S. patents, and U.S. provisional applications, as well as other compounds and substances that inhibit the erbB2 receptor, can be used with the compounds described herein in accordance with the present invention.

The compounds can also be used with other agents useful in treating abnormal cellular proliferation or cancer, including, but not limited to, agents capable of enhancing antitumor immune responses, such as CTLA4 (cytotoxic lymphocite antigen 4) antibodies, and other agents capable of blocking CTLA4; and anti-proliferative agents such as other farnesyl protein transferase inhibitors, and the like. Specific CTLA4 antibodies that can be used in the present invention include those described in U.S. Provisional Application 60/113,647 (filed Dec. 23, 1998) which is incorporated by reference in its entirety, however other CTLA4 antibodies can be used in the present invention.

Other anti-angiogenesis agents, including, but not limited to, other COX-II inhibitors, other MMP inhibitors, other anti-VEGF antibodies or inhibitors of other effectors of vascularization can also be used.

In another embodiment, the compounds, when used as an antiproliferative, can be administered in combination with another compound that increases the effectiveness of the therapy, including but not limited to an antifolate, a 5-fluoropyrimidine (including 5-fluorouracil), a cytidine analogue such as β-L-1,3-dioxolanyl cytidine or β-L-1,3-dioxolanyl 5-fluorocytidine, antimetabolites (including purine antimetabolites, cytarabine, fudarabine, floxuridine, 6-mercaptopurine, methotrexate, and 6-thioguanine), hydroxyurea, mitotic inhibitors (including CPT-11, Etoposide (VP-21), taxol, and vinca alkaloids such as vincristine and vinblastine, an alkylating agent (including but not limited to busulfan, chlorambucil, cyclophosphamide, ifofamide, mechlorethamine, melphalan, and thiotepa), non-classical alkylating agents, platinum containing compounds, bleomycin, an anti-tumor antibiotic, an anthracycline such as doxorubicin and daunomycin, an anthracenedione, topoisomerase II inhibitors, hormonal agents (including but not limited to corticosteroids (dexamethasone, prednisone, and methylprednisone), androgens such as fluoxymesterone and methyltestosterone, estrogens such as diethylstilbesterol, antiestrogens such as tamoxifen, LHRH analogues such as leuprolide, anti-androgens such as flutamide, aminoglutethimide, megestrol acetate, and medroxyprogesterone), asparaginase, carmustine, lomustine, hexamethyl-melamine, dacarbazine, mitotane, streptozocin, cisplatin, carboplatin, levamasole, and leucovorin. The compounds of the present invention can also be used in combination with enzyme therapy agents and immune system modulators such as an interferon, interleukin, tumor necrosis factor, macrophage colony-stimulating factor and colony stimulating factor. In one embodiment, the compounds described herein can be employed together with at least one other antiviral agent chosen from reverse transcriptase inhibitors, protease inhibitors, fusion inhibitors, entry inhibitors and polymerase inhibitors.

In addition, compounds according to the present invention can be administered in combination or alternation with one or more anti-retrovirus, anti-HBV, interferon, anti-cancer or antibacterial agents, including but not limited to other compounds of the present invention. Certain compounds described herein may be effective for enhancing the biological activity of certain agents according to the present invention by reducing the metabolism, catabolism or inactivation of other compounds, and as such, are co-administered for this intended effect.

### IX. PHARMACEUTICAL COMPOSITIONS

Hosts, including but not limited to humans, infected with a human immunodeficiency virus, a hepatitis B virus, or cancer can be treated by administering to the patient an effective amount of the active compound or a pharmaceutically acceptable prodrug or salt thereof in the presence of a pharmaceutically acceptable carrier or diluent. The active materials can be administered by any appropriate route, for example, orally, parenterally, intravenously, intradermally, subcutaneously, or topically, in liquid or solid form.

A preferred dose of the compound for will be in the range of between about 0.1 and about 100 mg/kg, more generally, between about 1 and 50 mg/kg, and, preferably, between about 1 and about 20 mg/kg, of body weight of the recipient per day. The effective dosage range of the pharmaceutically acceptable salts and prodrugs can be calculated based on the weight of the parent nucleoside to be delivered. If the salt or prodrug exhibits activity in itself, the effective dosage can be estimated as above using the weight of the salt or prodrug, or by other means known to those skilled in the art.

The compound is conveniently administered in unit any suitable dosage form, including but not limited to but not limited to one containing 7 to 3000 mg, preferably 70 to 1400 mg of active ingredient per unit dosage form. An oral dosage of 50-1000 mg is usually convenient.

Ideally the active ingredient should be administered to achieve peak plasma concentrations of the active compound from about 0.2 to 70 μM, preferably about 1.0 to 15 μM. This can be achieved, for example, by the intravenous injection of a 0.1 to 5% solution of the active ingredient, optionally in saline, or administered as a bolus of the active ingredient.

The concentration of active compound in the drug composition will depend on absorption, inactivation and excretion rates of the drug as well as other factors known to those of skill in the art. It is to be noted that dosage values will also vary with the severity of the condition to be alleviated. It is to be further understood that for any particular subject, specific dosage regimens should be adjusted over time according to the individual need and the professional judgment of the person administering or supervising the administration of the compositions, and that the concentration ranges set forth herein are exemplary only and are not intended to limit the scope or practice of the claimed composition. The active ingredient can be administered at once, or can be divided into a number of smaller doses to be administered at varying intervals of time.

A preferred mode of administration of the active compound is oral. Oral compositions will generally include an inert diluent or an edible carrier. They can be enclosed in gelatin capsules or compressed into tablets. For the purpose of oral therapeutic administration, the active compound can be incorporated with excipients and used in the form of tablets, troches or capsules. Pharmaceutically compatible binding agents, and/or adjuvant materials can be included as part of the composition.

The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring. When the dosage unit form is a capsule, it can contain, in addition to material of the above type, a liquid carrier such as a fatty oil. In addition, unit dosage forms can contain various other materials that modify the physical form of the dosage unit, for example, coatings of sugar, shellac, or other enteric agents.

The compound can be administered as a component of an elixir, suspension, syrup, wafer, chewing gum or the like. A syrup can contain, in addition to the active compound(s), sucrose as a sweetening agent and certain preservatives, dyes and colorings and flavors.

The compound or a pharmaceutically acceptable prodrug or salts thereof can also be mixed with other active materials that do not impair the desired action, or with materials that supplement the desired action, such as antibiotics, antifungals, anti-inflammatories or other antivirals, including but not limited to other nucleoside compounds. Solutions or suspensions used for parenteral, intradermal, subcutaneous, or topical application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents, such as ethylenediaminetetraacetic acid; buffers, such as acetates, citrates or phosphates, and agents for the adjustment of tonicity, such as sodium chloride or dextrose. The parental preparation can be enclosed in ampoules, disposable syringes or multiple dose vials made of glass or plastic.

If administered intravenously, preferred carriers are physiological saline or phosphate buffered saline (PBS).

In a preferred embodiment, the active compounds are prepared with carriers that will protect the compound against rapid elimination from the body, such as a controlled release formulation, including but not limited to implants and microencapsulated delivery systems. Biodegradable, biocompatible polymers can be used, such as ethylene vinyl acetate, polyanhydrides, polyglycolic acid, collagen, polyorthoesters and polylactic acid. For example, enterically coated compounds can be used to protect cleavage by stomach acid. Methods for preparation of such formulations will be apparent to those skilled in the art. Suitable materials can also be obtained commercially.

Liposomal suspensions (including but not limited to liposomes targeted to infected cells with monoclonal antibodies to viral antigens) are also preferred as pharmaceutically acceptable carriers. These can be prepared according to methods known to those skilled in the art, for example, as described in U.S. Pat. No. 4,522,811 (incorporated by reference). For example, liposome formulations can be prepared by dissolving appropriate lipid(s) (such as stearoyl phosphatidyl ethanolamine, stearoyl phosphatidyl choline, arachadoyl phosphatidyl choline, and cholesterol) in an inorganic solvent that is then evaporated, leaving behind a thin film of dried lipid on the surface of the container. An aqueous solution of the active compound or its monophosphate, diphosphate, and/or triphosphate derivatives is then introduced into the container. The container is then swirled by hand to free lipid material from the sides of the container and to disperse lipid aggregates, thereby forming the liposomal suspension.

The terms used in describing the invention are commonly used and known to those skilled in the art. As used herein, the following abbreviations have the indicated meanings:

aq aqueous
CDI carbonyldiimidazole

### DMF N,N-dimethylformamide

DMSO dimethylsulfoxide
EDC 1-ethyl-3-(3-dimethyllaminopropyl)carbodiimide hydrochloride
EtOAc ethyl acetate
h hour/hours

### HOBt N-hydroxybenzotriazole

M molar
min minute
rt or RT room temperature
TBAT tetrabutylammonium triphenyldifluorosilicate
TBTU O-(Benzotriazol-1-yl)-N,N,N′,N′-tetramethyluronium tetrafluoroborate
THF tetrahydrofuran

### X. GENERAL SCHEMES FOR PREPARING ACTIVE COMPOUNDS

Methods for the facile preparation of 6-substituted-2-amino purine dioxolane monophosphate and phosphonates prodrugs are also provided. The 6-substituted-2-amino purine dioxolane monophosphates and phosphonates prodrugs disclosed herein can be prepared as described in detail below, or by other methods known to those skilled in the art. It will be understood by one of ordinary skill in the art that these schemes are in no way limiting and that variations of detail can be made without departing from the spirit and scope of the present invention.

Generally, the nucleotides are prepared by first preparing the corresponding nucleoside, then capping the 5′-hydroxy group as a monophosphate or other analog as described herein that can be readily converted in vivo to an active triphosphate form of the compound.

The various reaction schemes are summarized below.

In one embodiment, the invention relates to a process for preparing the dioxolane compounds described herein. The process first involves preparing compounds of the general formula (1)

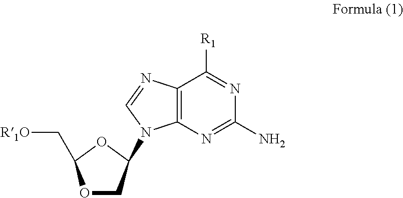

and pharmaceutically acceptable salts or prodrug thereof; wherein, R′₁ is a hydroxyl protecting group; and R₁ is as defined above,

by reacting a compound of the general formula (2)

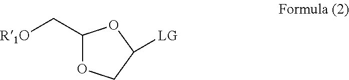

wherein LG is a leaving group as defined according to J. March, “Advanced Organic Chemistry”, 3rd edition, Wiley 1985,

with a 2,6-substituted purine derivative of the general formula (5)

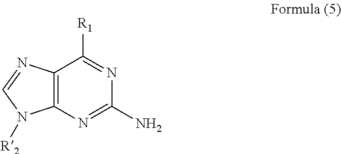

wherein; R′₂ is a silyl radical,

in the presence of a Lewis acid, solvent, and additionally in the presence of a 2-cyanoethanoate compound or a silylated derivative of a 2-cyanoethanoate compound.

After this step is completed, the hydroxyl protecting group R′₁ is removed, and the hydroxyl group is coupled to a phosphate or phosphonate group, or derivative thereof. The coupling step generally involves formation of a phosphate ester, wherein an activated phosphorous compound (i.e., containing a P—Cl bond, or other suitable bond with a leaving group) is reacted with the OH group to form HCl and the P—O linkage, or other suitable “H-leaving group” and the P—O linkage.

A representative phosphorous-containing reagent to couple with the —OH group is shown below:

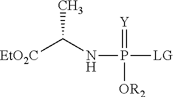

where R₂ is selected from the group consisting of C₁₋₈ alkyl, aryl, and heteroaryl, wherein the alkyl, aryl, and heteroaryl moieties can optionally substituted with from one to three substituents as described elsewhere herein as suitable substituents for such moieties;

LG is a leaving group, such as a halo (i.e., I, Br, Cl, or F), tosylate, brosylate, nosylate, mesylate, triflate, and the like, and

Y is O or S.

In one embodiment, the compound disclosed above includes a chiral phosphorus atom in enantiomerically-enriched form, so that the resulting prodrug also includes a chiral phosphorus atom in enantiomerically-enriched form.

A representative coupling reaction is shown below:

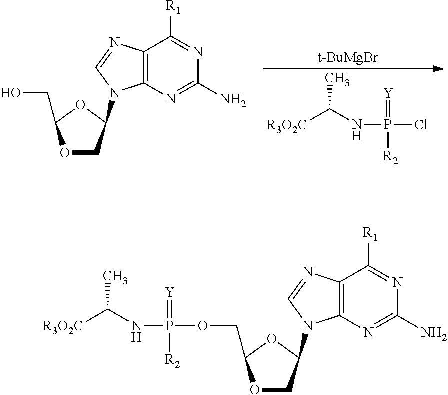

The process of the invention can be used to produce racemic prodrug compounds, or optically pure or enriched prodrug compounds, through choice of precursors having an appropriate optical configuration. If the phosphorus atom in the precursor used to prepare the phosphate or phosphonate prodrug is chiral, then appropriate diastereomers can be produced.

The hydroxyl protecting group R′₁ can be selected from all alcohol protecting group known and suitable to one skilled in the art. For example, alcohols protecting groups as described in “T. W. Greene, P. G. M. Wuts, “Protective Groups in Organic Synthesis”, 3^{rd} edition, Wiley 1999, pp. 17-200.

Leaving groups (“LG”) are preferably selected from iodine, bromine, C₁₋₂₀ acyloxy radical, C₁₋₂₀ alkylsulfonyloxy radical, C₁₋₂₀ arylsulfonyloxy radical, C₁₋₂₀ alkoxy radical and C₁₋₂₀ aryloxy radical.

The 2,6-disubstituted purine derivative of the general formula (5) contains at least one C₁₋₂₀ silyl radical R′₄, and optionally further silyl radicals on functions in positions 2 and 6, when possible, to act as amino protective groups.

The alpha cyano carbonyl compound used is a 2-cyanoethanoate ester, a 2-cyano ketone or a 2-cyanoethanoic acid derivative having 5 to 20 C atoms of the general formula (3)

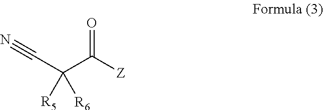

wherein Z may be hydrogen, an alkyl radical having from 1 to 20 C atoms, an aryl radical having from 6 to 20 C atoms or an alkyloxy group having from 1 to 20 C atoms and R₅ and R₆ can be, independently, a hydrogen, an acyl radical of an aromatic or aliphatic carboxylic acid having from 2 to 20 C atoms, an alkyl radical having from 1 to 20 C atoms or an aryl radical having from 6 to 20 C atoms.

The silylated derivative of 2-cyanoethanoate ester compound used is a silyl derivative of a 2-cyanoethanoate ester, of a 2-cyano ketone or of a 2-cyanoethanoic acid derivative of the general formula (4)

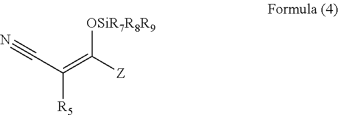

wherein Z and R₅ are as described above, and R₇, R₈ and R₉ may be independently of one another an aliphatic or aromatic radical having from 1 to 20 C atoms.

In general all aprotic organic solvents can be used for the process. The reaction is preferably carried out under atmospheric pressure at a temperature between −25° C. and the boiling point of the solvent.

The present invention also provides a recrystallization process for purifying compounds of the general formula (I) obtained by the process of the invention.

Preferred methods for removing OH protective acyl radical groups are reaction with ammonia, aliphatic amines, basic aqueous hydrolysis, or reaction with alcoholates. Preferred methods for forming the prodrugs described herein include reacting the resulting OH group with a phosphorus compound that includes a leaving group, such as chloride, that can be displaced by the OH group to form a P—O bond.

The first step of the process of the invention can be used to produce racemic compounds of general formula (1) and optically pure or enriched compounds obtained in the optical configuration of the general formulas (1a), (1b), (1c), or (1d)

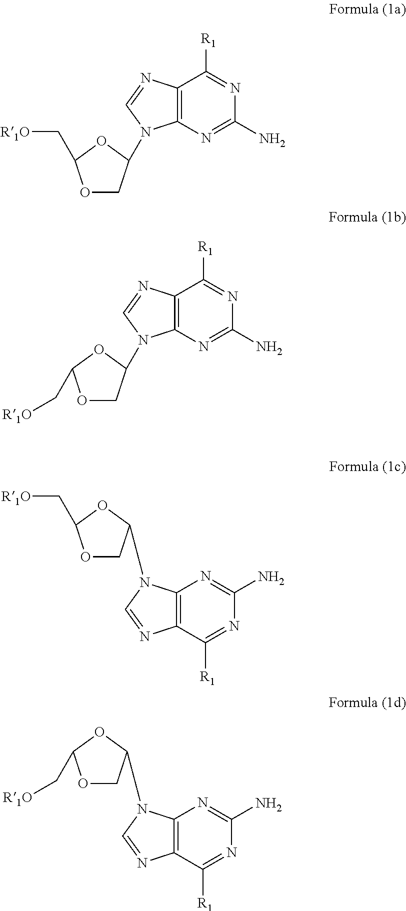

High stereoselectivity can be obtained by the process through choice of precursors having an appropriate optical configuration.

The hydroxyl protecting group R′₁ can be selected from all alcohol protecting group known and suitable to one skilled in the art. For example, alcohols protecting groups as described in “T. W. Greene, P. G. M. Wuts, “Protective Groups in Organic Synthesis”, 3^{rd} edition, Wiley 1999, pp. 17-200. The hydroxyl protective groups R₁ are preferably selected from the group comprising C₂₋₂₀ acyl radicals, C₁₋₂₀ alkyl radicals, C₁₋₂₀ alkoxyalkyl radicals, C₁₋₂₀ arylalkyl radicals, C₁₋₂₀ arylalkoxyalkyl radicals or C₁₋₂₀ silyl radicals.

Leaving groups LG are preferably selected from the group comprising iodine, bromine, C₁₋₂₀ acyloxy radical, C₁₋₂₀ alkylsulfonyloxy radical, C₁₋₂₀ arylsulfonyloxy radical, C₁₋₂₀ alkoxyradical or C₁₋₂₀ aryloxy radical. Particular preference is given for iodine and radicals from the group comprising acetoxy-, benzoyloxy-, propionyloxy-, n-butyryloxy- and trifluoroacetoxy-. Acetoxy- is very particularly preferred.

The 2,6-disubstituted purine derivative of the general formula (5) contains at least one C₁₋₂₀ silyl radical R₄, and optionally further silyl radicals on functions in positions 2 and 6, when possible, to act as amino protective groups. A persilylated precursor of the general formula (5) may in this connection comprise up to 5 identical or different silyl radicals. For example, 2,6-diaminopurine derivatives of the general formula (5) having one to three silyl radicals are preferred, and those having three silyl radicals are very particularly preferred, especially having silyl radical on the nitrogen in position 9 and a silyl radical on each of the two amino functions in positions 2 and 6. Trimethylsilyl- is particularly preferred.

Preferred Lewis acid compounds are selected from the group comprising trialkylsilylhalides or trialkylsilyl perfluoroalkanesulfonates. Iodotrimethylsilane and trimethylsilyl trifluoromethanesulfonate are particularly preferred.

The alpha cyano carbonyl compound used is a 2-cyanoethanoate ester, a 2-cyano ketone or a 2-cyanoethanoic acid derivative having 5 to 20 C atoms of the general formula (3)

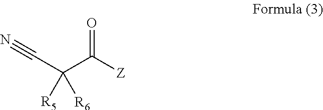

wherein Z may be hydrogen, an alkyl radical having from 1 to 20 C atoms, an aryl radical having from 6 to 20 C atoms or an alkyloxy group having from 1 to 20 C atoms and R₅ and R₆ may be independently of one another hydrogen, an acyl radical of an aromatic or aliphatic carboxylic acid having from 2 to 20 C atoms, an alkyl radical having from 1 to 20 C atoms or an aryl radical having from 6 to 20 C atoms.

The silylated derivative of 2-cyanoethanoate ester compound used is a silyl derivative of a 2-cyanoethanoate ester, of a 2-cyano ketone or of a 2-cyanoethanoic acid derivative of the general formula (4)

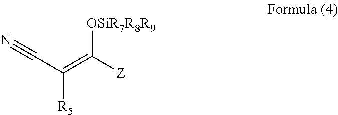

wherein Z and R₅ have the meaning set forth in claim 6, and R₇, R₈ and R₉ may be independently of one another an aliphatic or aromatic radical having from 1 to 20 C atoms.

In general all aprotic organic solvents can be used. Examples of suitable solvents are methylene chloride, 1,2-dichloroethane, and acetonitrile. Particularly preferred are methylene chloride and 1,2-dichloroethane.

The reaction is preferably carried out under atmospheric pressure at a temperature between −25° C. and the boiling point of the solvent. A temperature between −10° C. and +30° C. is preferably used.

The present invention also provides a recrystallization process for purifying compounds of the general formula (I) obtained by the process of the invention. Alcohols, ethers, or esters having 1-10 carbon atoms or other polar solvents are particularly suitable for the recrystallization. Isopropanol is particularly preferred as solvent for the recrystallization of compounds of the general formula (1) where R₁=(CH₃)₂CHCO—.

Preferred methods for removing OH protective acyl radical groups are reaction with ammonia, aliphatic amines, basic aqueous hydrolysis, or reaction with alcoholates such as, for example, sodium methoxide.

### EXAMPLES

The present invention is further illustrated in the following example. Scheme 1 shows the preparative method for synthesizing purine dioxolane nucleoside derivatives. It will be understood by one of ordinary skill in the art that these examples are in no way limiting and that variations of detail can be made without departing from the spirit and scope of the present invention. The 6′-NH₂ moiety can be replaced with another 6′-moiety, as described herein, for example, —Cl, —OMe, and —NH-cyclopropyl, without affecting the overall reaction scheme.

The terms used in describing the invention are commonly used and known to those skilled in the art. As used herein, the following abbreviations have the indicated meanings:

Ac acetyl

DMAP 4-dimethylaminopyridine

DMSO dimethylsulfoxide

h hour/hours

M molar

min minute

rt room temperature

TBDMSCl tert-butyl dimethyl silyl chloride

THF tetrahydrofuran

TMSI trimethylsilyl iodide

Specific compounds which are representative of this invention were prepared as per the following examples and reaction sequences; the examples and the diagrams depicting the reaction sequences are offered by way of illustration, to aid in the understanding of the invention and should not be construed to limit in any way the invention set forth in the claims which follow thereafter. The present compounds can also be used as intermediates in subsequent examples to produce additional compounds of the present invention. No attempt has necessarily been made to optimize the yields obtained in any of the reactions. One skilled in the art would know how to increase such yields through routine variations in reaction times, temperatures, solvents and/or reagents.

Anhydrous solvents were purchased from Aldrich Chemical Company, Inc. (Milwaukee). Reagents were purchased from commercial sources. Unless noted otherwise, the materials used in the examples were obtained from readily available commercial suppliers or synthesized by standard methods known to one skilled in the art of chemical synthesis. Melting points (mp) were determined on an Electrothermal digit melting point apparatus and are uncorrected. ¹H and ¹³C NMR spectra were taken on a Varian Unity Plus 400 spectrometer at room temperature and reported in ppm downfield from internal tetramethylsilane. Deuterium exchange, decoupling experiments or 2D-COSY were performed to confirm proton assignments. Signal multiplicities are represented by s (singlet), d (doublet), dd (doublet of doublets), t (triplet), q (quadruplet), br (broad), bs (broad singlet), m (multiplet). All J-values are in Hz. Mass spectra were determined on a Micromass Platform LC spectrometer using electrospray techniques. Elemental analyses were performed by Atlantic Microlab Inc. (Norcross, Ga.). Analytic TLC was performed on Whatman LK6F silica gel plates, and preparative TLC on Whatman PK5F silica gel plates. Column chromatography was carried out on Silica Gel or via reverse-phase high performance liquid chromatography.

### Example 1

### Preparation of DAPD

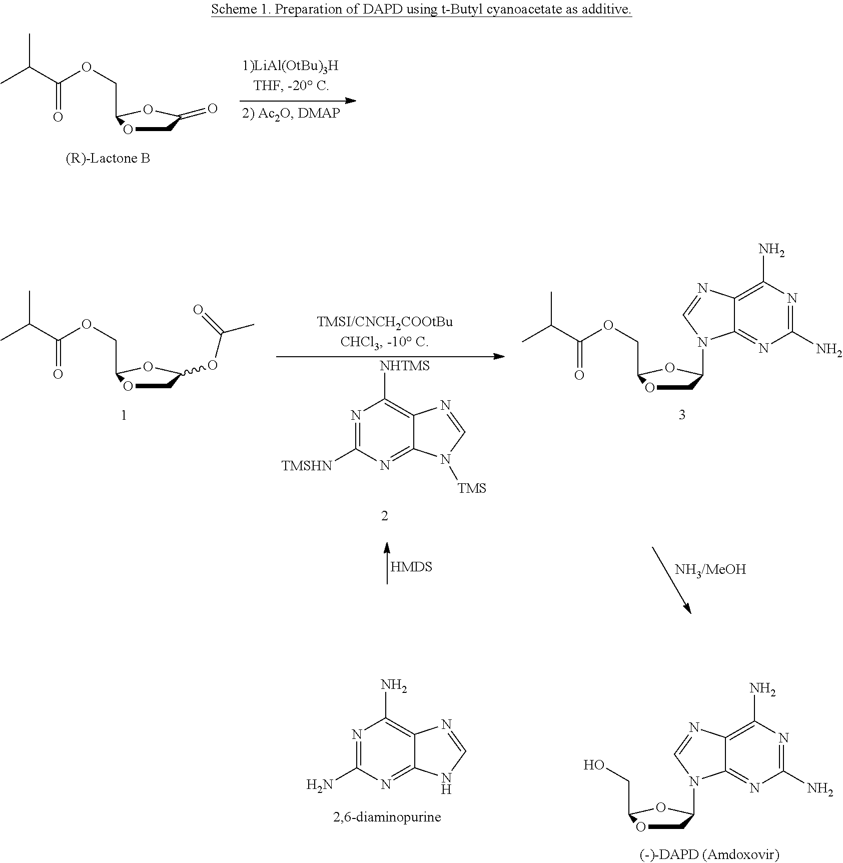

### Step 1: Silylation of 2,6-diaminopurine

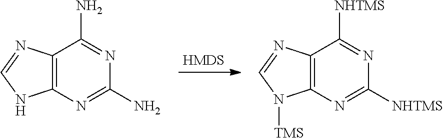

750 mg of 2,6-diaminopurine, 750 mg of ammonium sulfate and 20 mL of hexamethyldisilazane were added into a 250 mL three-neck flask. The suspension was heated to reflux with stirring at 130-135° C. (oil-bath) for 4 h. During this period the solution becomes homogeneous. The solution was cooled to 85° C. and the excess hexamethyldisilazane was subsequently distilled off under gradually decreasing reduced pressure. After the hexamethyldisilazane was removed completely, the residue was cooled to rt under vacuum then 10 mL of anhydrous methylene chloride was added to prepare a solution.

### Step 2: Preparation of (2R-4R/S)-4-acetoxy-2-isobutyryloxymethyl-1,3-dioxolane

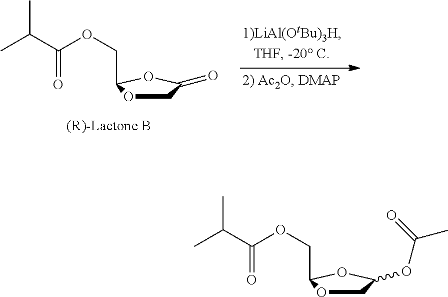

To a well stirred solution of LiAl(OtBu)₃H (25.4 g, 100 mmol) in dry THF (150 mL) at −10 to −20° C. was added a pre-cooled isobutyric acid-4-oxo-[1,3]-dioxolan-2-(R)-yl methyl ester (12.5 g, 66 mmol) over a period of 10 min under N₂ atmosphere. The reaction mixture was allowed to stir for 2 h at −10 to −20° C. To this solution DMAP (7.0 g, 57.4 mmol) was added in one portion and stirred for 30 min followed by dropwise addition of Ac₂O (46 mL, 443.3 mmol). After stirring the bright yellow solution for 2 h at −10° C., the cold bath was allowed to raise to room temperature and stirred overnight at rt. The dark brown solution was poured into saturated NH₄Cl (180 mL) solution, stirred for 30 min, filtered (to remove Li salt), concentrated in vacuo and extracted with ethyl acetate (3×60 mL). The combined organic solutions were washed with saturated NaHCO₃ (2×50 mL), brine, dried over Na₂SO₄, filtered, and evaporated under reduced pressure to afford a crude product (red syrup). R_{f}: 0.45 (ethylacetate:hexanes 1:4). NMR showed 1:1 mixture of α and β isomers. ¹H-NMR (CDCl₃) δ: 1.18 and 1.19 (2s, 6H, 2×CH₃); 2.10 (s, 3H, CH₃); 4.20-4.42 (m, 4H, 2×CH₂); 5.32 and 5.42 (t, 1H, J=4.4 Hz, CH); 6.41 and 6.35 (dd, 1H, J=4.0 Hz, J=1.6 Hz, CH).

Anhydrous CHCl₃ was added to a total volume of 40 mL to prepare a 1 M solution (based on a 60% yield)

### Step 3: Preparation of cis- and trans-(2R,4R)-2-isobutyryloxymethyl-4-(2,6-diaminopurin-9-yl)-[1,3]-dioxolane

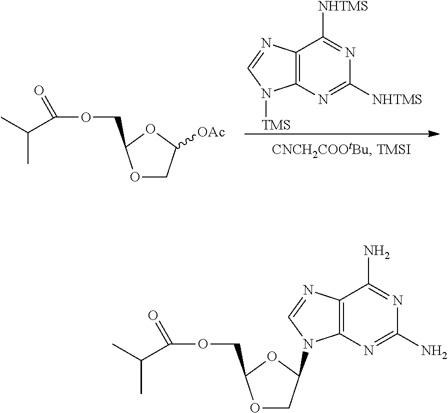

The solution of silylated 2,6-diaminopurine in dry methylene chloride (from step 1), 4 mL of 1M (2R-4R/S)-4-acetoxy-2-isobutyryloxymethyl-1,3-dioxolane solution in chloroform (from step 2) and 0.75 mL of t-butyl cyanoacetate were introduced into a dry flask. The mixture was cooled to 0 to −10° C. and, at this temperature, a solution of 1.5 mL of iodotrimethylsilane in 2 mL of methylene chloride was added dropwise over the course of 2-3 min. The mixture was then stirred at 0 to 5° C. for 20 h.

The reaction mixture was added dropwise to 18 mL of solution of 0.5 M hydrochloric acid at 0° C. The mixture was warmed with stirring to 25° C. and stirred for another 20 min. The phases were separated and the organic phase was back-extracted once with 18 mL of 0.5 M hydrochloric acid. The combined aqueous phases were washed twice with 25 mL of methylene chloride. Then, after addition of a further 50 mL of methylene chloride, the pH was adjusted to 9.0 with approximately 40 mL of 10% sodium carbonate solution. The mixture was stirred at 25° C. for 1 h and the phases were separated. The aqueous phase was back-extracted twice with 30 mL of methylene chloride. The combined organic phases were washed once with 25 mL of water. Removal of the solvent in vacuum resulted in 940 mg of yellowish solid. LCMS analysis showed the isomer ratio (β:α=2.2:1).

The crude product was recrystallized from isopropanol and 690 mg (45% yield) of colorless β-isomer crystals were obtained. NMR analysis revealed 1 mol of isopropanol in addition to (2R)-2-isobutyryloxymethyl-4-(2,6-diaminopurin-9-yl)-1,3-dioxolane. ¹H-NMR (DMSO-d₆) δ: 0.95-1.04 (m, 12H, 4×CH₃); 2.44-2.4 (m, 1H, CH); 3.71-3.75 (m, 1H), 4.17-4.52 (m, 5H, CH, 2×CH₂); 5.20 (t, 1H, J=3.2 Hz, CH); 5.80 (brs, 2H, NH₂) 6.17 (dd, 1H, J=1.6 Hz, J=5.6 Hz, CH); 6.71 (brs, 2H, NH₂); 7.74 (s, 1H, ArH).

### Step 4: Preparation of [(2R,4R)-[4-(2,6-diamino-9H-purin-9-yl)-1,3-dioxolan-2-yl]methanol [(−)-DAPD]

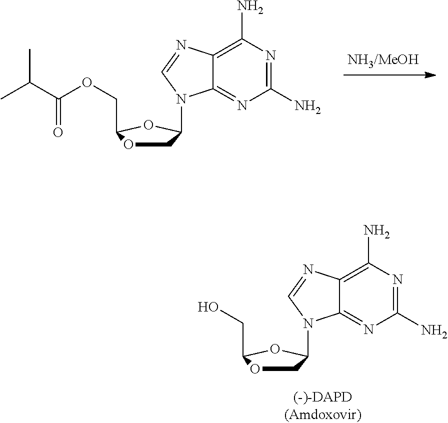

31.05 g of (2R,4R)-2-isobutyryloxymethyl-4-(2,6-diaminopurin-9-yl)-1,3-dioxolane.2-propanol was dissolved in 310 mL of NH₃-saturated methanol. The solution was stirred at 25° C. for 15 h and the solvent was distilled off in vacuo. The residue was recrystallized from ethanol/water. 17.10 g (83%) of (−)-DAPD were obtained as colorless crystals.

¹H-NMR (360 MHz, DMSO-d₆): δ=3.61 (dd, J₁=6.0 Hz, J₂=3.2 Hz; CH₂OH); 4.20 (dd, J₁=9.5 Hz, J₂=5.5 Hz; 1H—C(5′)); 4.45 (dd, J₁=9.5 Hz, J₂=1.8 Hz; 1H—C(5′)); 5.05 (Ψt, J=3.2 Hz; 1H—C(2′)); 5.15 (Ψt, J=6.0 Hz; CH₂OH); 5.83 (s; 2H—NH₂); 6.21 (dd, J₁=5.5 Hz, J₂=1.8 Hz; 1H—C(4′)); 5.83 (s; 2H—NH₂); 7.87 (s; 1H—C (8)).

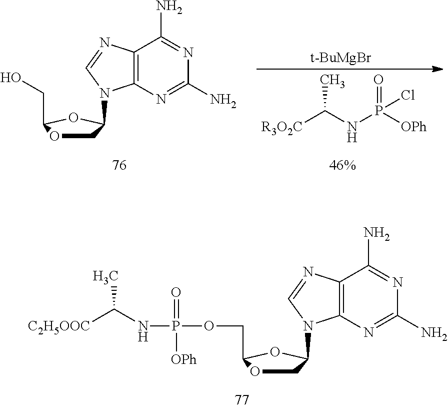

### Synthesis of 2,6-diamino purine dioxolane monophosphate Prodrug

### Example 2

### (2R)-ethyl-2-((((4R)-4-(2,6-diamino-9H-purin-9-yl)-1,3-dioxolan-2-yl)methoxy)(phenoxy)phosphorylamino)propanoate (77)

As shown above, to a solution of DAPD (30 mg, 0.12 mmol) in THF (5 mL) was added 1 M solution of t-BuMgCl (0.36 mL, 0.36 mmol) and stirred for 30 min. To the reaction mixture was added (2R)-ethyl 2-(chloro(phenoxy)phosphorylamino)propanoate (0.36 mL, 0.36 mmol) in THF at rt and was stirred overnight, neutralized with ammonium chloride_{(aq)}, conc, the crude mixture was purified by flash column chromatography with ethyl acetate:methanol=5:1 to give 77 (28 mg, 46%).

¹H-NMR (CD₃OD, 300 MHz) δ: 7.80-7.79 (s, 1H), 7.26-7.09 (m, 5H), 6.27 (m, 1H), 6.12 (brs, 2H), 5.25 (m, 3H), 4.47 (m, 2H), 4.22 (m, 2H), 4.03 (m, 2H), 3.83 (m, 1H), 1.33-1.15 (m, 6H).

LC/MS calcd. for C₂₀H₂₇N₇O₇P 508.2, observed: 508.3 (M+1).

The same chemistry can be used to prepare 6′-substituted analogs of DAPD, for example, those in which the 6′-position includes a halo (i.e., Cl, Br, I, or F), OMe, NH-cyclopropyl, or other suitable moiety, that, when the compounds are metabolized, is converted to an OH moiety. Instead of starting with DAPD, one would start with the 6′-substituted DAPD analog.

### Example 3

### Conversion of 6-substituted-2-amino purine dioxolanes to 6-hydroxy-2-amino purine dioxolanes

The various nucleosides prepared as described above, with functionality at the 6′-position other than a hydroxy group, are readily converted, in vivo, to the 6′-hydroxy form when the 5′-OH group is not converted to the monophosphate prodrug.

The metabolism of (−)-β-D-2,6-diaminopurine dioxolane (DAPD) in PHA-stimulated human PBMCs and CEM cells was previously assessed (Antimicrob. Agents Chemother. 2001, 45, 158-165). In this previous study DAPD was found to readily deaminate to (−)-β-D-dioxolane guanine (DXG). While both DXG and DAPD were detected, DAPD levels in PBMCs were 27-fold higher than the level of DAPD determined in CEM cells; the level of DXG was roughly the same in both cell types. The intracellular levels of DAPD and DXG and their phosphorylated derivatives were quantitated in the same previous study. No phosphorylation of DAPD to the corresponding mono-, di-, or triphosphate forms was detected in either cell type. It was shown that DAPD was deaminated to DXG and was subsequently phosphorylated to DXG-TP.

Reexamination of the intracellular metabolism of DAPD, which contains a 6-amino group, at 50 μM for 4 h in PBM cells at 37° C. resulted the detection of high levels of DXG-TP in addition to DXG and DXG-MP. Low levels of DAPD were observed however, no phosphorylated forms of DAPD were detected (FIG. 10).

Shown in the table below are the HIV and toxicity data for DAPD-MP prodrug RS-864 and the parent nucleoside DAPD. In this case an increase in anti-HIV activity for RS-864 is noted at both the EC₅₀, and EC₉₀ however there is also a slight increase in toxicity relative to the parent nucleoside DAPD.

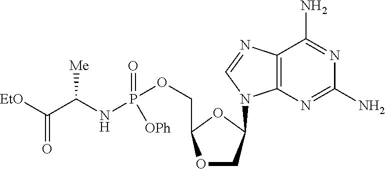

RS-864 (n=2; HIV assay)
HIV EC₅₀=0.24 μM
HIV EC₉₀=1.3 μM
PBM IC₅₀=66.5% @ 100 μM
CEM IC₅₀>100 μM
Vero IC₅₀>100 μM

### Parent nucleoside (DAPD)

EC₅₀/EC₉₀=1.0/6.5 μM
PBM IC₅₀=>100 μM
CEM IC₅₀>100 μM
Vero IC₅₀>100 μM

### HIV and Toxicity Data for MP Prodrug RS-864 and the Parent Nucleoside DAPD

Incubation of RS-864, which contains a 6-amino group and a 5′-MP prodrug, in PBM cells resulted in the detection of low levels of DXG, DXG-MP, and DXG-TP (FIG. 11). However, in contrast to the incubation of DAPD, very high levels of DAPD-TP were detected. In addition, low levels of DAPD, DAPD-MP, DAPD-DP were also observed. The high levels of intercellular DAPD-TP produced upon incubation of the DAPD-MP prodrug indicate that the MP prodrug has efficiently limited or stopped the conversion of the 6-amino group to 6-OH.

### Example 4

### Anti-HIV (in PBM cells) Assay

The biological activity of the compounds described herein is discussed below.

Anti-HIV-1 activity of the compounds was determined in human peripheral blood mononuclear (PBM) cells as described previously (see Schinazi R. F., McMillan A., Cannon D., Mathis R., Lloyd R. M. Jr., Peck A., Sommadossi J.-P., St. Clair M., Wilson J., Furman P. A., Painter G., Choi W.-B., Liotta D. C. Antimicrob. Agents Chemother. 1992, 36, 2423; Schinazi R. F., Sommadossi J.-P., Saalmann V., Cannon. D., Xie M.-Y., Hart G., Smith G., Hahn E. Antimicrob. Agents Chemother. 1990, 34, 1061). Stock solutions (20-40 mM) of the compounds were prepared in sterile DMSO and then diluted to the desired concentration in growth medium. Cells were infected with the prototype HIV-1_{LAI} at a multiplicity of infection of 0.01. Virus obtained from the cell supernatant was quantified on day 6 after infection by a reverse transcriptase assay using (rA)ₙ.(dT)₁₂₋₁₈ as template-primer. The DMSO present in the diluted solution (<0.1%) had no effect on the virus yield. AZT was included as positive control. The antiviral EC₅₀ and EC₉₀ were obtained from the concentration-response curve using the median effective method described previously (see Chou T.-. C. & Talalay P. Adv. Enzyme Regul. 1984, 22, 27-55; Belen'kii M. S. & Schinazi R. F. Antiviral Res. 1994, 25, 1-11).

### Example 5

### Assess Incorporation of Nucleoside-TPs by HIV-1 RT

i) Protein Expression and Purification: HIV-1 RT (xxLAI background) (see Shi C, Mellors J W. A recombinant retroviral system for rapid in vivo analysis of human immunodeficiency virus type 1 susceptibility to reverse transcriptase inhibitors. Antimicrob Agents Chemother. 1997; 41:2781-5) was over-expressed in bacteria using the p6HRT-PROT expression vector and purified to homogeneity as described previously (see Le Grice S F, Gruninger-Leitch F. Rapid purification of homodimer and heterodimer HIV-1 reverse transcriptase by metal chelate affinity chromatography. Eur J. Biochem. 1990; 187: 307-14; Le Grice S F, Cameron C E, Benkovic S J. Purification and characterization of human immunodeficiency virus type 1 reverse transcriptase. Methods Enzymol. 1995; 262:130-44). The protein concentration of the purified enzymes was determined spectrophotometrically at 280 nm using an extinction co-efficient (ε280) of 260450M−1 cm−1. Active site concentrations of RT were calculated from pre-steady-state burst experiments, as described previously (see Kati W M, Johnson K A, Jerva L F, Anderson K S. Mechanism and fidelity of HIV reverse transcriptase. J. Biol. Chem. 1992; 267: 25988-97). All reactions described below were carried out using active site concentrations.

ii) Pre-steady-state Kinetic Analyses: A [γ³²P]-ATP 5′-end labeled 20 nucleotide DNA primer (5′-TCGGGCGCCACTGCTAGAGA-3′) annealed to a 57 nucleotide DNA template (5′-CTCAGACCCTTTTAGTCAGAATGGAAANTCTCTAGCAGTGGCGCCCG AACAGGGACA-3′) was used in all experiments. The DNA templates contained either a T or C at position 30 (N), which allowed evaluation of the kinetics of single nucleotide incorporation using the same 20 nucleotide primer. Rapid quench experiments were carried out using a Kintek RQF-3 instrument (Kintek Corporation, Clarence, Pa.). In all experiments, 300 nM RT and 60 nM DNA template/primer (T/P) were pre-incubated in reaction buffer (50 mM Tris-HCl pH 7.5, 50 mM KCl) prior to mixing with an equivalent volume of nucleotide in the same reaction buffer containing 20 mM MgCl₂. Reactions were terminated at times ranging from 10 ms to 30 min by quenching with 0.5M EDTA, pH 8.0. The quenched samples were mixed with an equal volume of gel loading buffer (98% deionized formamide, 10 mM EDTA and 1 mg/mL each of bromophenol blue and xylene cyanol), denatured at 85° C. for 5 min, and the products were separated from the substrates on a 7M urea-16% polyacrylamide gel. Product formation was analyzed using a Bio-Rad GS525 Molecular Imager (Bio-Rad Laboratories, Inc., Hercules, Calif.).

iii) Data Analysis: Data obtained from kinetic assays was fitted by nonlinear regression using Sigma Plot software (Jandel Scientific) with the appropriate equations (see Johnson K A. Rapid quench kinetic analysis of polymerases, adenosinetriphosphatases, and enzyme intermediates. Methods Enzymol. 1995; 249:38-61). The apparent burst rate constant (kobs) for each particular concentration of dNTP was determined by fitting the time courses for the formation of product to the equation: [product]=A[1−exp(−kobst)], where A represents the burst amplitude. The turnover number (kpol) and apparent dissociation constant for dNTP (K_{d}) was obtained by plotting the apparent catalytic rates, kobs, against dNTP concentrations and fitting the data with the following hyperbolic equation: kobs=(kpol[dNTP])/([dNTP]+K_{d}).

### Example 6

### Assess Anti-HIV Activity and Cellular Toxicity of 6-Substituted-2-amino purine dioxolane monophosphate Prodrugs

i) Viruses: Stock virus was prepared using the xxHIV-1LAI clone75 by electroporating (Gene Pulser; Bio-Rad) 5 to 10 μg of plasmid DNA into 1.3×10⁷ MT-2 cells. At 7 days post-transfection, cell-free supernatant was harvested and stored at −80° C. The genotype of stock viruses was confirmed by extraction of RNA from virions, treatment of the extract with DNase I, amplification of the full-length coding region (amino acids 1 to 560) of RT by RT-PCR, purification of the PCR product, and sequence determination of the PCR product using a Big Dye terminator kit (v. 3.1) on an ABI 3100 automated DNA sequencer (Applied Biosystems, Foster City, Calif.). The 50% tissue culture infective dose (TCID₅₀) for the virus stock was determined for MT-2 cells, P4/R5 cells or PBM cells by three-fold endpoint dilution assays (six wells per dilution) and calculated using the Reed and Muench equation (see Reed L J, Muench H. A simple method of estimating fifty percent endpoints. Am. J. Hyg. 1938; 27:493-497).

ii) Single-Replication-Cycle Drug Susceptibility Assay: In a 96-well plate, two- or three-fold serial dilutions of an inhibitor were added to P4/R5 cells in triplicate. Cells were infected with the amount of virus that yielded a relative light unit value of 100 in the no-drug, virus-infected control wells. At 48 h post-infection, a cell lysis buffer and luminescent substrate (Gal-Screen; Tropix/Applied Biosystems) was added to each well, and relative light unit values were determined using a luminometer (ThermolabSystems, Waltham, Mass.). Inhibition of virus replication was calculated as the concentration of compound required to inhibit virus replication by 50% (EC₅₀).

iii) Multiple-Replication-Cycle Drug Susceptibility Assay: In a 96-well plate, three-fold serial dilutions of an inhibitor were added to MT-2 cells in triplicate. The cells were infected at a multiplicity of infection of 0.01 as determined by endpoint dilution in MT-2 cells. At 7 days post-infection, culture supernatants were harvested and treated with 0.5% Triton X-100. The p24 antigen concentration in the supernatants was determined using a commercial enzyme-linked immunosorbent assay (DuPont, NEN Products, Wilmington, Del.). EC₅₀ values were calculated as described above.

iv) Drug Susceptibility Assays in PBM Cells: PBM cells were isolated by Ficoll-Hypaque discontinuous gradient centrifugation from healthy seronegative donors, as described previously (see Schinazi R F, Cannon D L, Arnold B H, Martino-Saltzman D. Combinations of isoprinosine and 3′-azido-3′-deoxythymidine in lymphocytes infected with human immunodeficiency virus type 1. Antimicrob. Agents Chemother. 1988; 32:1784-1787; Schinazi R F, Sommadossi J P, Saalmann V, Cannon D L, Xie M Y, Hart G C, Smith G A. Hahn E. F. Activities of 3′-azido-3′-deoxythymidine nucleotide dimers in primary lymphocytes infected with human immunodeficiency virus type 1. Antimicrob. Agents Chemother. 1990; 34:1061-1067). Cells were stimulated with phytohemagglutinin A (PHA, Difco, Sparks, Md.) for 2-3 days prior to use. Infections were done in bulk for 1 h, either with 100 TCID₅₀/1×10⁷ cells for a flask (T25) assay or with 200 TCID₅₀/6×10⁷ cells/well for the 24-well plate assay. Cells were added to a plate or a flask containing a 10-fold serial dilution of the test compound. At 5 days post-infection, culture supernatants were harvested and treated with 0.5% Triton X-100. The p24 antigen concentration in the supernatants was determined as described above. EC₅₀ and fold-resistance values were calculated as described above.

v) Cellular Toxicity Assays: 6-Substituted-2-amino purine dioxolane monophosphate prodrugs were evaluated for their potential toxic effects on P4/R5 cells, MT-2 cells and uninfected PHA-stimulated human PBM cell. Log-phase P4/R5, MT-2, and PHA-stimulated human PBM cells were seeded at 5×10³ to 5×10⁴ cells/well in 96-well cell culture plates containing 10-fold serial dilutions of the test drug. The cultures were incubated for 2-4 days, after which 3-(4,5-dimethylthiazol-2-yl)-2,5-diphenyltetrazolium bromide (MIT) dye solution (Promega, Madison, Wis.) were added to each well and incubated overnight. The reaction was stopped with stop solubilization solution (Promega, Madison, Wis.) and plates were read at a wavelength of 570 nm. The median 50% cytotoxic concentration (CC₅₀) was determined from the concentration-response curve using the median effect method.

### Example 7

### Assess Activity of 6-Substituted-2-amino purine dioxolane monophosphate prodrugs against Drug-Resistant HIV

Analogs identified above as having improved activity compared with the parent analog, and less cellular toxicity, were further evaluated for activity against a panel of drug resistant viruses. The drug resistant viruses used in this study included HIV-1_{K65R}, HIV-1_{K70E}, HIV-1_{L74V}, HIV-1_{M184V}. HIV-1_{AZT2}, HIV-1_{AZT3}, HIV-1_{AZT7}, HIV-1_{AZT9}, HIV-1_{Q151M} and HIV-1_{69Insertion}. All of these mutant viruses were generated in our HIV-1xxLAI clone.

### Example 8

### Assess Activity of 6-Substituted-2-amino purine dioxolane monophosphate Prodrugs against Drug-Resistant HIV

i) Viruses and Drug Susceptibility Assays: Virus stocks were prepared as described above. Drug susceptibility assays were performed using the single- and multiple-replication-cycle assays also described above. Inhibition of virus replication was calculated as the concentration of compound required to inhibit virus replication by 50% (EC₅₀). Fold resistance values were determined by dividing the EC₅₀ for mutant HIV-1 by the EC₅₀ for WT HIV-1.

ii) Statistical analysis: To determine if fold-resistance values are statistically significant, EC₅₀ values from at least three independent experiments were log 10 transformed and compared using a two-sample Student's t test with Sigma Stat software (Jandel Scientific). P values less than 0.05 were considered to be statistically significant.

### Example 9

### Assess Incorporation and Excision of Nucleotides by Mutant HIV-1 RTs

i) Enzymes: The following mutant HIV-1 RT enzymes can be used in this study: K65R RT, K70E RT, L74V RT, M184V RT, AZT2 RT, AZT3 RT, Q151M RT and 69Insert RT. E. coli protein expression vectors for each of these mutant RTs can be developed, and protein expression and purification can be performed as described previously. Protein concentration and active site concentration is determined as described above.

ii) Kinetic Analyses of Nucleotide Incorporation: Pre-steady-state kinetic analyses can be used to determine the kinetic parameters Kd and kpol for each novel nucleoside-TPs for K65R, K70E RT, L74V RT, M184V RT and Q151M RT. Experimental design and data analysis can be carried out as described above.

iii) Excision Assays: The ATP-mediated phosphorolytic excision of the novel analogs from chain-terminated template/primer can be carried out using WT RT, AZT2 RT, AZT3 RT and 69Insert RT. The 20 nucleotide DNA primer described above can be 5′-end labeled with [γ³²P]-ATP and then annealed to the appropriate 57 nucleotide DNA template. The 3′-end of the primer can be chain-terminated by incubation with WT RT and 100 μM of the appropriate modified nucleotide analog for 30 min at 37° C. The ³²P-labeled, chain-terminated 21 nucleotide primer can be further purified by extraction of the appropriate band after 7M urea-16% acrylamide denaturing gel electrophoresis. The purified chain-terminated primer can then be re-annealed to the appropriate DNA template for use in phosphorolysis experiments. The phosphorolytic removal of nucleoside-MP can be achieved by incubating 300 nM (active site) WT or mutant RT with 60 nM of the chain-terminated T/P complex of interest in 50 mM Tris-HCl pH 8.0, 50 mM KCl. The reaction can be initiated by the addition of 3.0 mM ATP and 10 mM MgCl₂. Inorganic pyrophosphatase (0.01 U) can be present throughout the reaction. After defined incubation periods, aliquots can be removed from the reaction tube and quenched with equal volumes of gel loading dye (98% deionized formamide, 10 mM EDTA and 1 mg/mL each of bromophenol blue and xylene cyanol). Products can be separated by denaturing gel electrophoresis, and the disappearance of substrate coincident with formation of product can be analyzed using a Bio-Rad GS525 Molecular Imager. Data were fit to the following single exponential equation to determine the apparent rate (kATP) of ATP-mediated excision: [product]=A[exp(−kATPt)], where A represents the amplitude for product formation. Dead-end complex formation can be determined as described previously (see Meyer P R, Matsuura S E, Mian A M, So A G, Scott W A. A mechanism of AZT resistance: an increase in nucleotide-dependent primer unblocking by mutant HIV-1 reverse transcriptase. Mol. Cell. 1999; 4:35-43; Sluis-Cremer N, Arion D, Parikh U, Koontz D, Schinazi R F, Mellors J W, Parniak M A. The 3′-azido group is not the primary determinant of 3′-azido-3′-deoxythymidine (AZT) responsible for the excision phenotype of AZT-resistant HIV-1. J Biol. Chem. 2005; 280: 29047-52).

### Example 10

### Mitochondrial Toxicity Assays in HepG2 Cells

i) Effect of 6-Substituted-2-amino purine dioxolane monophosphate prodrugs on Cell Growth and Lactic Acid Production: The effect on the growth of HepG2 cells was determined by incubating cells in the presence of 0 μM, 0.1 μM, 1 μM, 10 μM and 100 μM drug. Cells (5×10⁴ per well) were plated into 12-well cell culture clusters in minimum essential medium with nonessential amino acids supplemented with 10% fetal bovine serum, 1% sodium pyruvate, and 1% penicillin/streptomycin and incubated for 4 days at 37° C. At the end of the incubation period the cell number was determined using a hemocytometer. Also taught by Pan-Zhou X-R, Cui L, Zhou X-J, Sommadossi Darley-Usmer V M. “Differential effects of antiretroviral nucleoside analogs on mitochondrial function in HepG2 cells” Antimicrob. Agents Chemother. 2000; 44: 496-503. To measure the effects of the nucleoside analogs on lactic acid production, HepG2 cells from a stock culture were diluted and plated in 12-well culture plates at 2.5×10⁴ cells per well. Various concentrations (0 μM, 0.1 μM, 1 μM, 10 μM and 100 μM) of nucleoside analog were added, and the cultures were incubated at 37° C. in a humidified 5% CO₂ atmosphere for 4 days. At day 4 the number of cells in each well were determined and the culture medium collected. The culture medium was filtered, and the lactic acid content in the medium determined using a colorimetric lactic acid assay (Sigma-Aldrich). Since lactic acid product can be considered a marker for impaired mitochondrial function, elevated levels of lactic acid production detected in cells grown in the presence of 6-substituted-2-amino purine dioxolane monophosphate prodrug analogs would indicate a drug-induced cytotoxic effect.

ii) Effect on 6-Substituted-2-amino purine dioxolane monophosphate prodrugs on Mitochondrial DNA Synthesis: a real-time PCR assay to accurately quantify mitochondrial DNA content has been developed (see Stuyver L J, Lostia S, Adams M, Mathew J S, Pai B S, Grier J, Tharnish P M, Choi Y, Chong Y, Choo H, Chu C K, Otto M J, Schinazi R F. Antiviral activities and cellular toxicities of modified 2′,3′-dideoxy-2′,3′-didehydrocytidine analogs. Antimicrob. Agents Chemother. 2002; 46: 3854-60). This assay was used in all studies described in this application that determine the effect of nucleoside analogs on mitochondrial DNA content. In this assay, low-passage-number HepG2 cells were seeded at 5,000 cells/well in collagen-coated 96-well plates. Dioxolane monophosphate analogs were added to the medium to obtain final concentrations of 0 μM, 0.1 μM, 10 μM and 100 μM culture day 7, cellular nucleic acids were prepared by using commercially available columns (RNeasy 96 kit; Qiagen). These kits co-purify RNA and DNA, and hence, total nucleic acids were eluted from the columns. The mitochondrial cytochrome c oxidase subunit II (COXII) gene and the β-actin or rRNA gene were amplified from 5 μl of the eluted nucleic acids using a multiplex Q-PCR protocol with suitable primers and probes for both target and reference amplifications. For COXII the following sense, probe and antisense primers are used, respectively: 5′-TGCCCGCCATCATCCTA-3′,5′-tetrachloro-6-carboxyfluorescein-TCCTCATCGCCCTCCCATCCC-TAMRA-3′ and 5′-CGTCTGTTATGTAAAGGATGCGT-3′. For exon 3 of the β-actin gene (GenBank accession number E01094) the sense, probe, and antisense primers are 5′-GCGCGGCTACAGCTTCA-3′, 5′-6-FAMCACCACGGCCGAGCGGGATAMRA-3′ and 5′-TCTCCTTAATGTCACGCACGAT-3′, respectively. The primers and probes for the rRNA gene are commercially available from Applied Biosystems. Since equal amplification efficiencies were obtained for all genes, the comparative CT method was used to investigate potential inhibition of mitochondrial DNA synthesis. The comparative CT method uses arithmetic formulas in which the amount of target (COXII gene) is normalized to the amount of an endogenous reference (the β-actin or rRNA gene) and is relative to a calibrator (a control with no drug at day 7). The arithmetic formula for this approach is given by 2-ΔΔCT, where ΔΔCT is (CT for average target test sample−CT for target control)−(CT for average reference test−CT for reference control) (see Johnson M R, K Wang, J B Smith, M J Heslin, R B Diasio. Quantitation of dihydropyrimidine dehydrogenase expression by real-time reverse transcription polymerase chain reaction. Anal. Biochem. 2000; 278:175-184). A decrease in mitochondrial DNA content in cells grown in the presence of drug would indicate mitochondrial toxicity.

iii) Electron Microscopic Morphologic Evaluation: NRTI induced toxicity has been shown to cause morphological changes in mitochondria (e.g., loss of cristae, matrix dissolution and swelling, and lipid droplet formation) that can be observed with ultrastructural analysis using transmission electron microscopy (see Cui L, Schinazi R F, Gosselin G, Imbach J L. Chu C K, Rando R F, Revankar G R, Sommadossi J P. Effect of enantiomeric and racemic nucleoside analogs on mitochondrial functions in HepG2 cells. Biochem. Pharmacol. 1996, 52, 1577-1584; Lewis W, Levine E S, Griniuviene B, Tankersley K O, Colacino J M, Sommadossi J P, Watanabe K A, Perrino F W. Fialuridine and its metabolites inhibit DNA polymerase gamma at sites of multiple adjacent analog incorporation, decrease mtDNA abundance, and cause mitochondrial structural defects in cultured hepatoblasts. Proc Natl Acad Sci USA. 1996; 93: 3592-7; Pan-Zhou X R, L Cui, X J Zhou, J P Sommadossi, V M Darley-Usmar. Differential effects of antiretroviral nucleoside analogs on mitochondrial function in HepG2 cells. Antimicrob. Agents Chemother. 2000, 44, 496-503). For example, electron micrographs of HepG2 cells incubated with 10 μM fialuridine (FIAU; 1,2′-deoxy-2′-fluoro-1-D-arabinofuranosly-5-iodo-uracil) showed the presence of enlarged mitochondria with morphological changes consistent with mitochondrial dysfunction. To determine if 6-substituted-2-amino purine dioxolane monophosphate prodrugs promoted morphological changes in mitochondria, HepG2 cells (2.5×10⁴ cells/mL) were seeded into tissue cultures dishes (35 by 10 mm) in the presence of 0 μM, 0.1 μM, 1 μM, 10 μM and 100 μM nucleoside analog. At day 8, the cells were fixed, dehydrated, and embedded in Eponas described previously. Thin sections were prepared, stained with uranyl acetate and lead citrate, and then examined using transmission electron microscopy.

### Example 11

### Mitochondrial Toxicity Assays in Neuro2A Cells

To estimate the potential of nucleoside analogs to cause neuronal toxicity, mouse Neuro2A cells (American Type Culture Collection 131) can be used as a model system (see Ray A S, Hernandez-Santiago B I, Mathew J S, Murakami E, Bozeman C, Xie M Y, Dutschman G E, Gullen E, Yang Z, Hurwitz S, Cheng Y C, Chu C K, McClure H, Schinazi R F, Anderson K S. Mechanism of anti-human immunodeficiency virus activity of beta-D-6-cyclopropylamino-2′,3′-didehydro-2′,3′-dideoxyguanosine. Antimicrob. Agents Chemother. 2005, 49, 1994-2001). The concentrations necessary to inhibit cell growth by 50% (CC₅₀) can be measured using the 3-(4,5-dimethyl-thiazol-2-yl)-2,5-diphenyltetrazolium bromide dye-based assay, as described. Perturbations in cellular lactic acid and mitochondrial DNA levels at defined concentrations of drug can be carried out as described above. In all experiments, ddC and AZT can be used as control nucleoside analogs.

### Example 12

### Effect of Nucleotide Analogs on the DNA Polymerase and Exonuclease Activities of Mitochondrial DNA Polymerase γ

i) Purification of Human Polymerase γ: The recombinant large and small subunits of polymerase γ can be purified as described previously (see Graves S W, Johnson A A, Johnson K A. Expression, purification, and initial kinetic characterization of the large subunit of the human mitochondrial DNA polymerase. Biochemistry. 1998, 37, 6050-8; Johnson A A, Tsai Y, Graves S W, Johnson K A. Human mitochondrial DNA polymerase holoenzyme: reconstitution and characterization. Biochemistry 2000; 39: 1702-8). The protein concentration can be determined spectrophotometrically at 280 nm, with extinction coefficients of 234,420, and 71,894 M−1 cm⁻¹ for the large and the small subunits of polymerase γ, respectively.

ii) Kinetic Analyses of Nucleotide Incorporation: Pre-steady-state kinetic analyses can be carried out to determine the catalytic efficiency of incorporation (k/K) for DNA polymerase γ for nucleoside-TP and natural dNTP substrates. This allows determination of the relative ability of this enzyme to incorporate modified analogs and predict toxicity. Pre-steady-state kinetic analyses of incorporation of nucleotide analogs by DNA polymerase γ can be carried out essentially as described previously (see Murakami E, Ray A S, Schinazi R F, Anderson K S. Investigating the effects of stereochemistry on incorporation and removal of 5-fluorocytidine analogs by mitochondrial DNA polymerase gamma: comparison of D- and L-D4FC-TP. Antiviral Res. 2004, 62, 57-64; Feng J Y, Murakami E, Zorca S M, Johnson A A, Johnson K A, Schinazi R F, Furman P A, Anderson K S. Relationship between antiviral activity and host toxicity: comparison of the incorporation efficiencies of 2′,3′-dideoxy-5-fluoro-3′-thiacytidine-triphosphate analogs by human immunodeficiency virus type 1 reverse transcriptase and human mitochondrial DNA polymerase. Antimicrob Agents Chemother. 2004, 48, 1300-6). Briefly, a pre-incubated mixture of large (250 nM) and small (1.25 mM) subunits of polymerase γ and 60 nM DNA template/primer in 50 mM Tris-HCl, 100 mM NaCl, pH 7.8, can be added to a solution containing MgCl₂ (2.5 mM) and various concentrations of nucleotide analogs. Reactions can be quenched and analyzed as described previously. Data can be fit to the same equations as described above.

iii) Assay for Human Polymerase γ 3′5′ Exonuclease Activity: The human polymerase γ exonuclease activity can be studied by measuring the rate of formation of the cleavage products in the absence of dNTP. The reaction can be initiated by adding MgCl₂ (2.5 mM) to a pre-incubated mixture of polymerase γ large subunit (40 nM), small subunit (270 nM), and 1,500 nM chain-terminated template/primer in 50 mM Tris-HCl, 100 mM NaCl, pH 7.8, and quenched with 0.3M EDTA at the designated time points. All reaction mixtures can be analyzed on 20% denaturing polyacrylamide sequencing gels (8M urea), imaged on a Bio-Rad GS-525 molecular image system, and quantified with Molecular Analyst (Bio-Rad). Products formed from the early time points can be plotted as a function of time. Data were fitted by linear regression with Sigma Plot (Jandel Scientific). The slope of the line can be divided by the active enzyme concentration in the reaction to calculate the kexo for exonuclease activity (see Murakami E, Ray A S, Schinazi R F, Anderson K S. Investigating the effects of stereochemistry on incorporation and removal of 5-fluorocytidine analogs by mitochondrial DNA polymerase gamma: comparison of D- and L-D4FC-TP. Antiviral Res. 2004; 62: 57-64; Feng J Y, Murakami E, Zorca S M, Johnson A A, Johnson K A, Schinazi R F, Furman P A, Anderson K S. Relationship between antiviral activity and host toxicity: comparison of the incorporation efficiencies of 2′,3′-dideoxy-5-fluoro-3′-thiacytidine-triphosphate analogs by human immunodeficiency virus type 1 reverse transcriptase and human mitochondrial DNA polymerase. Antimicrob Agents Chemother. 2004; 48: 1300-6).

### Example 13

### Assay for Bone Marrow Cytotoxicity

Primary human bone marrow mononuclear cells were obtained commercially from Cambrex Bioscience (Walkersville, Md.). CFU-GM assays were carried out using a bilayer soft agar in the presence of 50 units/mL human recombinant granulocyte/macrophage colony-stimulating factor, while BFU-E assays used a methylcellulose matrix containing 1 unit/mL erythropoietin (see Sommadossi J P, Carlisle R. Toxicity of 3′-azido-3′-deoxythymidine and 9-(1,3-dihydroxy-2-propoxymethyl) guanine for normal human hepatopoietic progenitor cells in vitro. Antimicrob. Agents Chemother. 1987; 31: 452-454; Sommadossi, J P, Schinazi, R F, Chu, C K, and Xie, M Y. Comparison of Cytotoxicity of the (−) and (+) enantiomer of 2′,3′-dideoxy-3′-thiacytidine in normal human bone marrow progenitor cells. Biochem. Pharmacol. 1992; 44:1921-1925). Each experiment was performed in duplicate in cells from three different donors. AZT was used as a positive control. Cells were incubated in the presence of the compound for 14-18 days at 37° C. with 5% CO₂, and colonies of greater than 50 cells are counted using an inverted microscope to determine IC₅₀. The 50% inhibitory concentration (IC₅₀) was obtained by least-squares linear regression analysis of the logarithm of drug concentration versus BFU-E survival fractions. Statistical analysis was performed with Student's t test for independent non-paired samples.

### Example 14

### Anti-HBV Assay

The anti-HBV activity of the compounds was determined by treating the AD-38 cell line carrying wild type HBV under the control of tetracycline (see Ladner S. K., Otto M. J., Barker C. S., Zaifert K., Wang G. H., Guo J. T., Seeger C. & King R. W. Antimicrob. Agents Chemother. 1997, 41, 1715-20). Removal of tetracycline from the medium [Tet (−)] results in the production of HBV. The levels of HBV in the culture supernatant fluids from cells treated with the compounds were compared with that of the untreated controls. Control cultures with tetracycline [Tet (+)] were also maintained to determine the basal levels of HBV expression. 3TC was included as positive control.

### Example 15

### Cytotoxicity Assay

The toxicity of the compounds can be assessed in Vero, human PBM, CEM (human lymphoblastoid), MT-2, and HepG2 cells, as described previously (see Schinazi R. F., Sommadossi J.-P., Saalmann V., Cannon D. L., Xie M.-Y., Hart G. C., Smith G. A. & Hahn E. F. Antimicrob. Agents Chemother. 1990, 34, 1061-67). Cycloheximide can be included as positive cytotoxic control, and untreated cells exposed to solvent can be included as negative controls. The cytotoxicity IC₅₀ can be obtained from the concentration-response curve using the median effective method described previously (see Chou T.-C. & Talalay P. Adv. Enzyme Regul. 1984, 22, 27-55; Belen'kii M. S. & Schinazi R. F. Antiviral Res. 1994, 25, 1-11).

### Example 16

### Adenosine Deaminase Assay

To determine the propensity for deamination of the 6-substituted-2-amino purine dioxolane monophosphate prodrugs by adenosine deaminase, nucleoside compounds were incubated with the commercially available purified enzyme, and the reaction was followed spectrophotometrically. Reaction conditions were 50 mM potassium phosphate, pH 7.4, with 50 μM nucleoside analog in 0.5 mL at 25° C. Reaction time was 7 minutes with 0.002 units of enzyme and 120 minutes with 0.2 units of enzyme. (The unit definition of adenosine deaminase is one unit will deaminate 1.0 μmol of adenosine to inosine per minute at pH 7.5 at 25° C.) Deoxyadenosine was the positive control which was 59% deaminated under the given conditions in 7 minutes with 0.002 units of enzyme. Deoxyguanosine was the negative control. Optical density was measured at 265 nm or 285 nm. The difference in optical density between the beginning and the end of the experiment was divided by the extinction coefficient then multiplied by the volume of the reaction to determine the number of mols of substrate transformed into product. Mols of product were divided by mols of substrate equivalent to a 100% complete reaction then multiplied by 100 to obtain percent deamination. The limit of detection was 0.001 optical density units.

### Example 17

### Selection of Resistant Viruses to Nucleotide Monophosphate Prodrugs

Peripheral blood mononuclear (PBM) cells¹ can be seeded at 1×10⁷ cells in a total of 5 mL of RPMI-1640 (Mediatech Inc., Herndon, Va.) containing 100 mL heat inactivated fetal bovine serum (Hyclone, Logan, Utah), 83.3 IU/mL penicillin, 83.3 μg/mL streptomycin (Mediatech Inc., Herndon, Va.), 1.6 mM L-glutamine (Mediatech Inc., Herndon, Va.), 0.0008% DEAE-Dextran (Sigma-Aldrich, St. Louis, Mo.), 0.047% sodium bicarbonate, and 26 IU/mL recombinant interleukin-2 (Chiron Corporation, Emeryville, Calif.) in two T25 flask, one control (untreated) and one treated with drug. ¹ PBM cells can be separated by ficoll-hypaque (Histopaque 1077: Sigma) density gradient centrifugation from Buffy coats obtained from the American Red Cross (Atlanta, Ga.). Buffy coats can be derived from healthy, seronegative donors. Cells can be activated with 3 μg/mL phytohemagglutinin A (Sigma-Aldrich, St. Louis, Mo.) in 500 mL of RPMI-1640 (Mediatech Inc., Herndon, Va.) containing 100 mL heat inactivated fetal bovine serum (Hyclone, Logan, Utah), 83.3 IU/mL penicillin, 83.3 μg/mL streptomycin, 1.6 mM L-glutamine (Mediatech Inc., Herndon, Va.), for 2-3 days prior to use.

Naive PBM cells can be treated with nucleotide monophosphate prodrug at 0.1 μM for one hour prior to inoculation with HIV-1_{LAI}² at 100×TCID₅₀. The treated PBM cell group and a control nontreated PBM cell group can be allowed to infect, for example, for one hour. An additional 5 mL RTU medium can be added to each flask and cells can be incubated, for example, for 6 days at 37° C. ² HIV-1/LAI can be obtained from the Center for Disease Control and Prevention and used as the virus for the resistant pool and a multiplicity of infection (MOI) of 0.1, as determined by a limiting dilution method in PBM cells, can be selected to begin the infected pool.

On day 6, 1 mL of supernatant from each flask can be removed and spun at 9,740 g at 4° C. for 2 hr. The resulting viral pellet can then be resuspended in virus solubilization buffer for RT analysis. Total RNA can be isolated from culture supernatants using the commercial QIAmp Viral RNA mini kit (Quiagen). Sequencing can be performed in parallel between the control virus and nucleotide monophosphate prodrug treated virus to determine if there are any mutations created by the applied drug pressure on weeks where the virus appears to be resistant.

The percent inhibition of the treated viral pool relative to the untreated viral pool can be calculated and closely monitored weekly prior to treatment. The selective pressure for the viral pool can be increased from 0.1 μM to 3.5 μM (40 times the EC₅₀ value) over a period of as many as 47 weeks or more.

### Example 18

### Synthesis of Nucleoside Analog Triphosphates

Nucleoside analog triphosphates were synthesized from the corresponding nucleosides, using the Ludwig and Eckstein's method. (Ludwig J, Eckstein F. “Rapid and efficient synthesis of nucleoside 5′-O-(1-thiotriphosphates), 5′-triphosphates and 2′,3′-cyclophosphorothioates using 2-chloro-4H-1,3,2-benzodioxaphosphorin-4-one” J. Org. Chem. 1989, 54 631-5) The crude nucleoside analog triphosphate can be purified, for example, by FPLC using a HiLoad 26/10 Q Sepharose Fast Flow Pharmacia column and gradient of TEAB buffer (pH 7.0). The product will be characterized by UV spectroscopy, proton and phosphorus NMR, mass spectroscopy and HPLC.

The resulting triphosphates can be used as controls for the cellular pharmacology assays described above and for kinetic work with HIV-RT (for example, 6-substituted-2-amino purine dioxolane triphosphate with HIV-RT).

### Example 19

### Phosphorylation Assay of Nucleoside to Active Triphosphate in HepG2 cells

To determine the cellular metabolism of the compounds, HepG2 cells can be obtained from the American Type Culture Collection (Rockville, Md.), and can be grown in 225 cm² tissue culture flasks in minimal essential medium supplemented with non-essential amino acids, 1% penicillin-streptomycin. The medium is renewed every three days, and the cells are sub-cultured once a week. After detachment of the adherent monolayer with a 10 minute exposure to 30 mL of trypsin-EDTA and three consecutive washes with medium, confluent HepG2 cells can be seeded at a density of 2.5×10⁶ cells per well in a 6-well plate and exposed to 10 μM of [³H] labeled active compound (500 dpm/pmol) for the specified time periods.

The cells are maintained at 37° C. under a 5% CO₂ atmosphere. At the selected time points, the cells are washed three times with ice-cold phosphate-buffered saline (PBS).

Intracellular active compound and its respective metabolites are extracted by incubating the cell pellet overnight at −20° C. with 60% methanol followed by extraction with an additional 20 pal of cold methanol for one hour in an ice bath. The extracts are then combined, dried under gentle filtered air flow and stored at −20° C. until HPLC analysis.

### Example 20

### Bioavailability Assay in Cynomolgus Monkeys

The following procedure can be used to determine whether the compounds are bioavailable. Within 1 week prior to the study initiation, a cynomolgus monkey can be surgically implanted with a chronic venous catheter and subcutaneous venous access port (VAP) to facilitate blood collection and can undergo a physical examination including hematology and serum chemistry evaluations and the body weight recording. Each monkey (six total) receives approximately 250 μCi of ³H activity with each dose of active compound at a dose level of 10 mg/kg at a dose concentration of 5 mg/mL, either via an intravenous bolus (3 monkeys, IV), or via oral gavage (3 monkeys, PO). Each dosing syringe is weighed before dosing to gravimetrically determine the quantity of formulation administered. Urine samples are collected via pan catch at the designated intervals (approximately 18-0 hours pre-dose, 0-4, 4-8 and 8-12 hours post-dosage) and processed. Blood samples are collected as well (pre-dose, 0.25, 0.5, 1, 2, 3, 6, 8, 12 and 24 hours post-dosage) via the chronic venous catheter and VAP or from a peripheral vessel if the chronic venous catheter procedure should not be possible. The blood and urine samples are analyzed for the maximum concentration (Cmax), time when the maximum concentration is achieved (TmaX), area under the curve (AUC), half life of the dosage concentration (TV,), clearance (CL), steady state volume and distribution (Vss) and bioavailability (F).

### Example 21

### Cell Protection Assay (CPA)

The assay is performed essentially as described by Baginski, S. G.; Pevear, D. C.; Seipel, M.; Sun, S. C. C.; Benetatos, C. A.; Chunduru, S. K.; Rice, C. M. and M. S. Collett “Mechanism of action of a pestivirus antiviral compound” PNAS USA 2000, 97 (14), 7981-7986. MDBK cells (ATCC) are seeded onto 96-well culture plates (4,000 cells per well) 24 hours before use. After infection with BVDV (strain NADL, ATCC) at a multiplicity of infection (MOI) of 0.02 plaque forming units (PFU) per cell, serial dilutions of test compounds are added to both infected and uninfected cells in a final concentration of 0.5% DMSO in growth medium. Each dilution is tested in quadruplicate.

Cell densities and virus inocula are adjusted to ensure continuous cell growth throughout the experiment and to achieve more than 90% virus-induced cell destruction in the untreated controls after four days post-infection. After four days, plates are fixed with 50% TCA and stained with sulforhodamine B. The optical density of the wells is read in a microplate reader at 550 nm.

The 50% effective concentration (EC₅₀) values are defined as the compound concentration that achieved 50% reduction of cytopathic effect of the virus.

### Example 22

### Plaque Reduction Assay

For a compound, the effective concentration is determined in duplicate 24-well plates by plaque reduction assays. Cell monolayers are infected with 100 PFU/well of virus. Then, serial dilutions of test compounds in MEM supplemented with 2% inactivated serum and 0.75% of methyl cellulose are added to the monolayers. Cultures are further incubated at 37° C. for 3 days, then fixed with 50% ethanol and 0.8% Crystal Violet, washed and air-dried. Then plaques are counted to determine the concentration to obtain 90% virus suppression.

### Example 23

### Yield Reduction Assay

For a compound, the concentration to obtain a 6-log reduction in viral load is determined in duplicate 24-well plates by yield reduction assays. The assay is performed as described by Baginski, S. G.; Pevear, D. C.; Seipel, M.; Sun, S. C. C.; Benetatos, C. A.; Chunduru, S. K.; Rice, C. M. and M. S. Collett “Mechanism of action of a pestivirus antiviral compound” PNAS USA 2000, 97 (14), 7981-7986, with minor modifications.

Briefly, MDBK cells are seeded onto 24-well plates (2×10⁵ cells per well) 24 hours before infection with BVDV (NADL strain) at a multiplicity of infection (MOI) of 0.1 PFU per cell. Serial dilutions of test compounds are added to cells in a final concentration of 0.5% DMSO in growth medium. Each dilution is tested in triplicate. After three days, cell cultures (cell monolayers and supernatants) are lysed by three freeze-thaw cycles, and virus yield is quantified by plaque assay. Briefly, MDBK cells are seeded onto 6-well plates (5×10⁵ cells per well) 24 h before use. Cells are inoculated with 0.2 mL of test lysates for 1 hour, washed and overlaid with 0.5% agarose in growth medium. After 3 days, cell monolayers are fixed with 3.5% formaldehyde and stained with 1% crystal violet (w/v in 50% ethanol) to visualize plaques. The plaques are counted to determine the concentration to obtain a 6-log reduction in viral load.

### Example 24

### Screening Method for Identifying Anti-Cancer Compounds

A representative screening method for identifying anti-cancer compounds is described in Skehan et al., Journal of the National Cancer Institute, Vol. 82, No. 13, 1107-1112, Jul. 4, 1990.

The method in Skehan measures the cellular protein content of adherent and suspension cultures in 96-well microtiter plates, and is suitable for ordinary laboratory purposes and for very large-scale applications.

Cultures are fixed with trichloroacetic acid and stained for 30 minutes with 0.4% (wt/vol) sulfurhodamine B (SRB) dissolved in 1% acetic acid. Unbound dye is removed by four washes with 1% acetic acid, and protein-bound dye is extracted with 10 mM un-buffered Tris base [tris(hydroxymethyl)aminomethane] for determination of optical density in a computer-interfaced, 96-well microtiter plate reader.

The SRB assay results are linear with the number of cells and with values for cellular protein measured by both the Lowry and Bradford assays at densities ranging from sparse subconfluence to multilayered supraconfluence.

The signal-to-noise ratio at 564 nm is approximately 1.5 with 1,000 cells per well. The sensitivity of the SRB assay compares favorably with sensitivities of several fluorescence assays and is purportedly superior to those of both the Lowry and Bradford assays and to those of 20 other visible dyes. The SRB assay provides a colorimetric end point that is nondestructive, indefinitely stable, and visible to the naked eye. It provides a sensitive measure of drug-induced cytotoxicity, is useful in quantitating clonogenicity, and is well suited to high-volume, automated drug screening. SRB fluoresces strongly with laser excitation at 488 nm and can be measured quantitatively at the single-cell level by static fluorescence cytometry.

### Example 25

### Comparative Data Showing the anti-HIV and anti-HBV Efficacy of Purine Dioxolane Compounds, and Counterpart Prodrugs

A series of experiments were performed, comparing various purine dioxolanes and their counterpart prodrugs, where the prodrugs were formed by attaching a

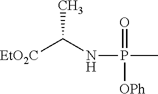

moiety to the 5′-hydroxy group.

Anti-HIV activity of C6 modified purine prodrugs (the particular prodrug is known as a ProTide) in human PBM cells were 3-250-fold more potent than DAPD and displayed 1-70-fold more potency than the DAPD metabolite DXG. The data is shown below in Table 1.

TABLE 1
Anti-HIV activity and cytotoxicity of C6 modified ProTides.

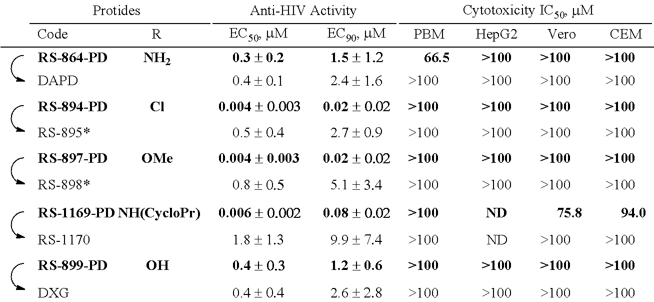

*Blue arrows indicate the parent drugs, and ProTides are in bold. ND, not deteremined.
*No apparent cytotoxicity was observed for all dioxolane nucleosides tested in HepG2, PBM, Vero, and CEM cells.

Modest anti-HBV activity was demonstrated for the parent C-6 modified nucleoside analogs (the data is shown in Table 2).

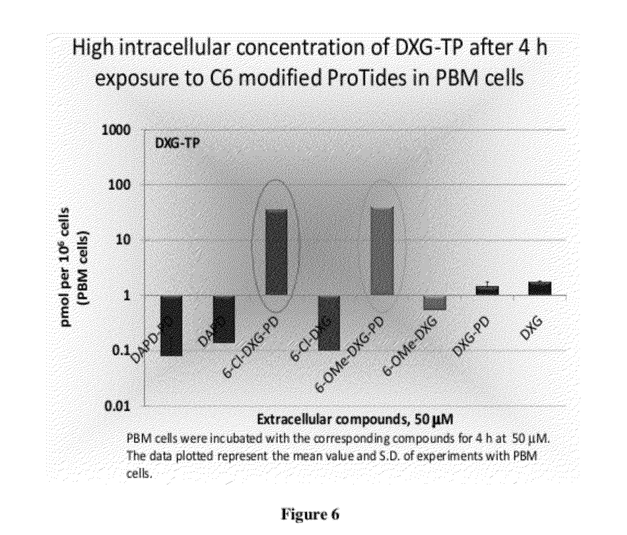

The corresponding ProTides were at least 15-fold more potent against HBV than DAPD, and at least 37-fold more potent than the DAPD deaminated metabolite DXG (Table 2 and FIG. 6).

TABLE 2
Anti-HBV activity of C6 modified ProTides.

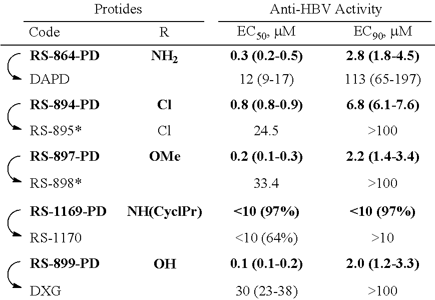

**Compounds tested singly in duplicate. Blue arrows indicate the parent drugs,
and ProTides are in bold.
*Modest anti-HBV activity was demonstrated for the C-6 modified parent
nucleoside analogs, whereas the prodrugs were markedly more potent.

In PBM cells, the intracellular levels of the active metabolite (DXG-TP) were on average 75-350 fold higher for C6 modified ProTides than the levels achieved with the parent nucleoside analogs (data shown in Table 3).

TABLE 3

| | DXG-TP levels: |
|---|---|
| Code | pmol/10⁶ cells |
| DAPD-PD | 0.08 ± 0.09 |
| DAPD | 0.14 ± 0.003 |
| 6-Cl-DXG-PD | 35.17 |
| 6-Cl-DXG | 0.10 |
| 6-OMe-DXG-PD | 41.02 |
| 6-OMe-DXG | 0.55 |
| DXG-PD | 1.41 ± 0.34 |
| DXG | 1.75 ± 0.07 |

PBM cells were incubated with the corresponding compounds for 4 h at 50 mM. The data plotted represent the mean value and S.D. of experiments with PBM cells.

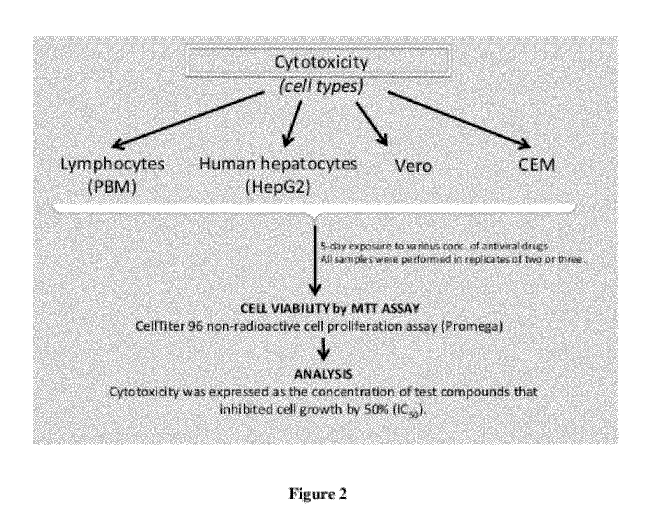

Of particular interest, as shown in FIG. 2, is that, after a four hour exposure to C6-modified ProTides in PBM cells, the prodrug forms of the 6-chloro and the 6-OMe analogs of DAPD showed a tremendously high intracellular concentration of the active metabolite (DXG-TP).

The anti-HBV activity was measured according to the procedures of Examples 10 and 19, by measuring the activity of the compounds in HepG2 cells. As shown in FIG. 6, in HepG2 cells, the intracellular levels of the active metabolite (DXG-TP) were, on average, around 130-500 fold higher for C6-modified ProTides than the levels achieved with the parent nucleoside drugs. As with the anti-HIV activity, the prodrug forms of the 6-chloro and the 6-OMe analogs of DAPD produced a tremendously intracellular concentration of the active metabolite in HepG2 cells, when the compounds were incubated with the cells for 4 hours at a concentration of 50 um.

In HepG2 cells, the levels of the active metabolite (DXG-TP) were on average 130-500 fold higher for C6 modified ProTides (a specific type of prodrug) than the levels achieved with the parent nucleoside analogs.

The data show that the ProTide approach alone, and/or in combination with modification at the C6 position of the purine ring, resulted in a marked enhancement of the anti-HIV and anti-HBV activity compared to the parent nucleoside analogs.

Understanding the relationship between structural modifications and the ability to confer simultaneous activity against these viruses can elucidate novel structure-activity relationships that can be used to design potent, safe antiviral agents for treatment of HIV infected, and HIV/HBV co-infected individuals.

Numerous references have been cited in this document. Each of these references is hereby incorporated by reference in its entirety.

While the foregoing specification teaches the principles of the present invention, with examples provided for the purpose of illustration, it will be understood that the practice of the invention encompasses all of the usual variations, adaptations and/or modifications as come within the scope of the following claims and their equivalents.

1. A compound of one of the following formulas: 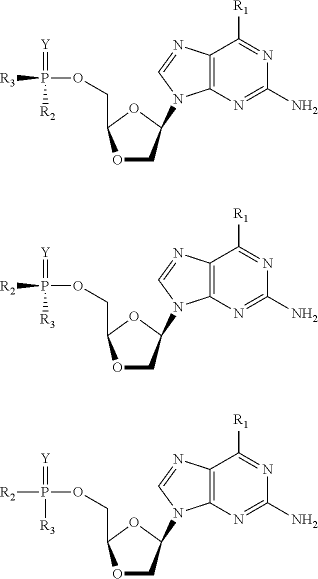
or a pharmaceutically acceptable salt or prodrug thereof, wherein:
R¹ is an atom or group removed in vivo to form OH when administered as the parent nucleoside, for example, halogen (F, Cl, Br, I), OR′, N(R′)₂, SR′, OCOR′, NHCOR′, N(COR′)COR′, SCOR′, OCOOR′, and NHCOOR′,
each R′ is independently H, a lower alkyl (C₁-C₆), lower haloalkyl (C₁-C₆), lower alkoxy (C₁-C₆), lower alkenyl (C₂-C₆), lower alkynyl (C₂-C₆), lower cycloalkyl (C₃-C₆) aryl, heteroaryl, alkylaryl, or arylalkyl, wherein the groups can be substituted with one or more substituents as defined above, for example, hydroxyalkyl, aminoalkyl, and alkoxyalkyl,
Y is O or S;
R² and R³, when administered in vivo, are ideally capable of providing the nucleoside monophosphate monophosphonate, thiomonophosphonate, or thiomonophosphate. Representative R² and R³ are independently selected from:
(a) OR⁸ where R⁸ is H, C₁₋₂₀ alkyl, C₃₋₆ cycloalkyl, C₁₋₆ haloalkyl, aryl, or heteroaryl which includes, but is not limited to, phenyl or naphthyl optionally substituted with one to three substituents independently selected from the group consisting of C₁₋₆ alkyl, C₂₋₆ alkenyl, C₂₋₆ alkynyl, C₁₋₆ alkoxy, (CH₂)₁₋₆CO₂R^{9a}, halogen, C₁₋₆ haloalkyl, —N(R^{9a})₂, C₁₋₆ acylamino, —NHSO₂C₁₋₆ alkyl, —SO₂N(R^{9a})₂, —SO₂C₁₋₆ alkyl, COR^{9b}, nitro and cyano;
R^{9a} is independently H or C₁₋₆ alkyl;
R^{9b} is —OR^{9a} or —N(R^{9a})₂;
(b) 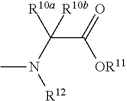
where R^{10a} and R^{10b} are:
(i) independently selected from the group consisting of H, C₁₋₁₀ alkyl, —(CH₂)ᵣNR^{9a}₂, C₁₋₆ hydroxyalkyl, —CH₂SH, —(CH₂)₂S(O)ₚMe, —(CH₂)₃NHC(═NH)NH₂, (1H-indol-3-yl)methyl, (1H-imidazol-4-yl)methyl, —(CH₂)ₘCOR^{9b}, aryl and aryl-C₁₋₃ alkyl, said aryl groups optionally substituted with a group selected from the group consisting of hydroxyl, C₁₋₁₀ alkyl, C₁₋₆ alkoxy, halogen, nitro, and cyano;
(ii) R^{10a} is H and R^{10b} and R¹² together are (CH₂)₂₋₄ to form a ring that includes the adjoining N and C atoms;
(iii) R^{10a} and R^{10b} together are (CH₂)ₙ to form a ring;
(iv) R^{10a} and R^{10b} both are C₁₋₆ alkyl; or
(v) 10^{a} is H and R^{10b} is H, CH₃, CH₂CH₃, CH(CH₃)₂, CH₂CH(CH₃)₂, CH(CH₃)CH₂CH₃, CH₂Ph, CH₂-indol-3-yl, —CH₂CH₂SCH₃, CH₂CO₂H, CH₂C(O)NH₂, CH₂CH₂COOH, CH₂CH₂C(O)NH₂, CH₂CH₂CH₂CH₂NH₂—CH₂CH₂CH₂NHC(NH)NH₂, CH₂-imidazol-4-yl, CH₂OH, CH(OH)CH₃, CH₂((4′-OH)-Ph), CH₂SH, or lower cycloalkyl;
p is 0 to 2;
r is 1 to 6;
n is 4 or 5;
m is 0 to 3;
R¹¹ is H, C₁₋₁₀ alkyl, or C₁₋₁₀ alkyl substituted with a lower alkyl, alkoxy, di(lower alkyl)-amino, fluoro, C₃₋₁₀ cycloalkyl, cycloalkyl alkyl, cycloheteroalkyl, aryl, such as phenyl, heteroaryl, such as, pyridinyl, substituted aryl, or substituted heteroaryl; wherein the substituents are C₁₋₅ alkyl, or C₁₋₅ alkyl substituted with a lower alkyl, alkoxy, di(lower alkyl)-amino, fluoro, C₃₋₁₀ cycloalkyl, or cycloalkyl;
R¹² is H, C₁₋₃ alkyl, or R^{10a}, or R^{10b} and R¹² together are (CH₂)₂₋₄ so as to form a ring that includes the adjoining N and C atoms;
(c) an O attached lipid (including a phospholipid), an N or O attached peptide, an O attached cholesterol, or an O attached phytosterol;
(d) R² and R³ may come together to form a ring 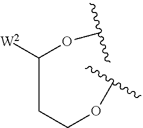
where W² is selected from a group consisting of phenyl or monocyclic heteroaryl, optionally substituted with one to three substituents independently selected from the group consisting of C₁₋₆ alkyl, CF₃, C₂₋₆ alkenyl, C₁₋₆ alkoxy, OR^{9c}, CO₂R^{9a}, COR^{9a}, halogen, C₁₋₆ haloalkyl, —N(R^{9a})₂, C₁₋₆ acylamino, CO₂N(R^{9a})₂, SR^{9a}, —NHSO₂C₁₋₆ alkyl, —SO₂N(R^{9a})₂, —SO₂C₁₋₆ alkyl, COR^{9b}, and cyano, and wherein said monocyclic heteroaryl and substituted monocyclic heteroaryl has 1-2 heteroatoms that are independently selected from the group consisting of N, O, and S with the provisos that:
a) when there are two heteroatoms and one is O, then the other can not be O or S, and
b) when there are two heteroatoms and one is S, then the other can not be O or S;
R^{9a} is independently H or C₁₋₆ alkyl;
R^{9b} is —OR^{9a} or —N(R^{9a})₂;
R^{9c} is H or C₁₋₆ acyl;
(e) 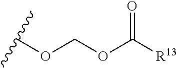
where R¹³ is selected from a group consisting of H, C₁₋₁₀ alkyl, C₁₋₁₀ alkyl optionally substituted with a lower alkyl, alkoxy, di(lower alkyl)-amino, fluoro, C₃₋₁₀ cycloalkyl, cycloalkyl alkyl, cycloheteroalkyl, aryl, such as phenyl, heteroaryl, such as, pyridinyl, substituted aryl, or substituted heteroaryl; wherein the substituents are C₁₋₅ alkyl, or C₁₋₅ alkyl substituted with a lower alkyl, alkoxy, di(lower alkyl)-amino, fluoro, C₃₋₁₀ cycloalkyl, or cycloalkyl;
f) R² and R³ may come together to form a ring 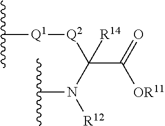
where R¹⁴ is: (i) independently selected from the group consisting of H, C₁₋₁₀ alkyl, —(CH₂)ᵣNR₂^{9a}, C₁₋₆ hydroxyalkyl, —CH₂SH, —(CH₂)₂S(O)ₚMe, —(CH₂)₃NHC(═NH)NH₂, (1H-indol-3-yl)methyl, (1H-imidazol-4-yl)methyl, —(CH₂)ₘCOR^{9b}, aryl and aryl-C₁₋₃ alkyl or heteroaryl and heteroaryl-C₁₋₃ alkyl, said aryl and heteroaryl groups optionally substituted with a group selected from the group consisting of hydroxyl, C₁₋₁₀ alkyl, C₁₋₆ alkoxy, halogen, nitro, and cyano; (ii) R¹⁴ is H, CH₃, CH₂CH₃, CH(CH₃)₂, CH₂CH(CH₃)₂, CH(CH₃)CH₂CH₃, CH₂Ph, CH₂-indol-3-yl, —CH₂CH₂SCH₃, CH₂CO₂H, CH₂C(O)NH₂, CH₂CH₂COOH, CH₂CH₂C(O)NH₂, CH₂CH₂CH₂CH₂NH₂, CH₂CH₂CH₂NHC(NH)NH₂, CH₂-imidazol-4-yl, CH₂OH, CH(OH)CH₃, CH₂((4′-OH)-Ph), CH₂SH, or lower cycloalkyl;
p is 0 to 2;
r is 1 to 6;
m is 0 to 3
Q¹ is NR^{9a}, O, or S
Q2 is C₁₋₁₀ alkyl, C₁₋₆ hydroxyalkyl, aryl and aryl-C₁₋₃ alkyl, heteroaryl and heteroaryl-C₁₋₃ alkyl, said aryl and heteroaryl groups optionally substituted with a group selected from the group consisting of hydroxyl, C₁₋₁₀ alkyl, C₁₋₆ alkoxy, fluoro, and chloro;
R¹¹ is H, C₁₋₁₀ alkyl, C₁₋₁₀ alkyl optionally substituted with a lower alkyl, alkoxy, di(lower alkyl)-amino, fluoro, C₃₋₁₀ cycloalkyl, cycloalkyl alkyl, cycloheteroalkyl, aryl, such as phenyl, heteroaryl, such as, pyridinyl, substituted aryl, or substituted heteroaryl; wherein the substituents are C₁₋₅ alkyl, or C₁₋₅ alkyl substituted with a lower alkyl, alkoxy, di(lower alkyl)-amino, fluoro, C₃₋₁₀ cycloalkyl, or cycloalkyl;
R¹² is H, or C₁₋₃ alkyl, or R^{14b} and R¹² together are (CH₂)₂₋₄ so as to form a ring that includes the adjoining N and C atoms;

2. The compound of claim 1, wherein one of R₂ and R₃ is

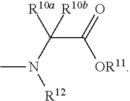

3. The compound of claim 2, wherein R¹² is H, one of R^{10a} and R^{10b} is methyl, and R¹¹ is C₁₋₁₀ alkyl.

4. The compound of claim 2, wherein R₃ is phenyl.

5. The compound of claim 3, wherein R₃ is phenyl.

6. The compound of claim 1, wherein R₁ is selected from the group consisting of halo, NH₂, OMe, and NH—C₃₋₆ cycloalkyl.

7. The compound of claim 1, wherein R₁ is selected from the group consisting of Cl, NH₂, OMe, and NH—C₃ cycloalkyl.

8. The compound of claim 1, wherein the compound is in the β-D-configuration.

9. The compound of claim 1, having one of the following formulas: 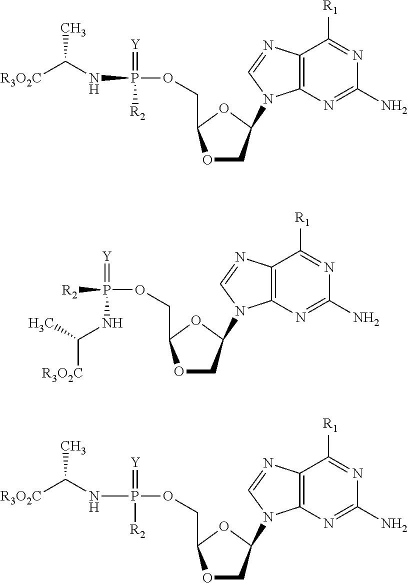
wherein R₁, R₂, and R₃ are as defined in claim 1.

10. The compound of claim 1, wherein the phosphorus atom is a chiral phosphorus atom, in enantiomerically-enriched form.

11. The compound of claim 10, wherein the chiral phosphorous is present in greater than 95% enantiomeric excess.

12. The compound of claim 1, wherein the compounds are in the β-D configuration.

13. A method for treating a host infected with HIV-1 or HIV-2, or reducing the biological activity of an HIV-1 or HIV-2 infection in a host, comprising administering an effective amount of a compound of claim 1 to a patient in need of treatment thereof.

14. A method for preventing an HIV-1 or HIV-2 infection, comprising administering an prophylactically-effective amount of a compound of claim 1 to a patient in need of prophylaxis thereof.

15. The method of claim 13, wherein the HIV-1 or HIV-2 infection is caused by a virus comprising a mutation selected from the group consisting of TAM mutations, the K65R mutation, and the M184V mutation.

16. The method of claim 13, wherein an effective amount of a compound of claim 1 is administered in combination with an additional anti-HIV agent.

17. The method of claim 16, wherein the additional anti-HIV agent is selected from the group consisting of AZT and 3TC.

18. The method of claim 16, wherein the additional anti-HIV agent is AZT, and the AZT is administered at a dosage at which it, in combination with the compound of claim 1, is effective in treating HIV, but at a dosage at which it is less likely to cause side effects than the conventional dosage of 300 mg/bid.

19. The method of claim 18, wherein the AZT is administered at a dosage of 250 mg/bid or less.

20. The method of claim 18, wherein the AZT is administered at a dosage of around 200 mg/bid.

21. The method of claim 18, wherein the HIV-1 or HIV-2 infection is caused by a virus comprising a mutation selected from the group consisting of TAM mutations, the K65R mutation, and the M184V mutation.

22. A method for treating a host infected with HBV, or reducing the biological activity of an HBV infection in a host, comprising administering an effective amount of a compound of claim 1 to a patient in need of treatment thereof.

23. The method of claim 22, wherein the effective amount of a compound of claim 1 is administered in combination with another anti-HBV agent.
